(19) United States
(12) Patent Application Publication
(10) Pub. No.: US 2003/0232836 A1
(43) Pub. Date: Dec. 18, 2003

(54) Acetamides and benzamides that are useful in treating sexual dysfunction

(76) Inventors: Andrew O. Stewart, Libertyville, IL (US); Teodozyj Kolasa, Lake Villa, IL (US)

Correspondence Address: STEVEN F. WEINSTOCK, ABBOTT LABORATORIES, 100 ABBOTT PARK ROAD, DEPT. 377/AP6A, ABBOTT PARK, IL, 60064-6008, US

(21) Appl. No.: 10/154,373

(22) Filed: May 23, 2002

(51) Int. Cl. A61K 31/513; A61K 31/506; A61K 31/501; A61K 31/496; A61K 31/495; A61K 31/4545; A61K 31/454

(52) U.S. Cl. 514/252.02; 514/252.03; 514/269; 514/252.14; 514/253.01; 514/253.11; 514/254.05; 514/254.02; 514/255.03; 514/318; 514/326; 514/317

(57) ABSTRACT

The present invention relates to the use of compounds of formula (I)

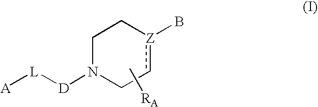

for the treatment of sexual dysfunction and to compositions containing compounds of formula (I) for the treatment of sexual dysfunction.

### TECHNICAL FIELD

The present invention relates to the use of acetamides and benzamides and compositions containing these compounds for the treatment of sexual dysfunction.

### BACKGROUND OF THE INVENTION

Preclinical evidence indicates that dopamine (DA) plays a role in penile erection in mammals. Sexual stimulation can be initiated by sensory (erotic) information reaching the cerebral cortex in mammals. The cerebral cortex has extensive neuronal connections with limbic structures like the amygdala, as well as midbrain structures like the periaqueductal gray (PAG) and the hypothalamus. Two important nuclei in the hypothalamus are the medial preoptic area (MPOA) and the paraventricular nucleus (PVN). The MPOA and PVN nuclei play a critical role in sexual behavior as bilateral lesions of these areas completely eliminate male sexual behavior. The incerto-hypothalamic dopaminergic pathway that innervates the PVN and the MPOA nuclei has been associated with the pro-erectile effect of DA agents. Systemic administration of DA receptor agonists like apomorphine ((6aR) 5,6,6a,7-tetrahydro-6-methyl-4H-dibenzo[de,g]quinoline-10,11-diol), quinpirole and (−) 3-(3-hydroxyphenyl)-N-propylpiperidine (3-PPP) facilitate penile erection in rats, an effect blocked by haloperidol, a central DA antagonist. As the erectogenic effect can not be blocked by domperidone, a peripheral DA antagonist, it is believed that the pro-erectile effect of DA agonists is centrally mediated.

Clinical data also indicates that DA systems in the CNS play a role on the regulation of male sexual behavior as indicated by the sexual stimulatory effect of L-dopa in Parkinson's patients and by the pro-erectile effect of apomorphine in humans.

DA receptors belong to a superfamily of protein receptors that signal across the cell membrane by coupling to intracellular GTP-binding proteins. Several G proteins have been identified (including Gs, Gq and Gi) that lead to specific intracellular events.

There are five known DA receptors which are classified into two groups, D₁-like and D₂-like. The D₁-like receptors include D₁ and D₅. The D₂-like receptors include D₂, D₃ and D₄. The D₁-like family receptor subtypes are Gₛ-coupled and can activate adenylate cyclase. The D₂-like family receptor subtypes are Gᵢ-coupled and they increase intracellular calcium level and inhibit adenylate cyclase.

The D₁-like family members are Gₛ-coupled receptors that can activate adenylate cyclase. The D₁ receptor is the most abundant and widespread DA receptor in the CNS both by mRNA expression and by immunohistochemical studies. It is found in the striatum, nucleus accumbens and olfactory tubercle as well as the limbic system, hypothalamus and thalamus. The D₁ receptor expression has been reported in the heart and kidney, and despite that the function of these peripheral D₁ receptors remains to be clarified, its role on the control of hemodynamic variables has been confirmed. The D₅ receptor, while having a higher affinity for DA than the D₁ receptor, is sparsely distributed in the CNS with no evidence of expression outside the CNS.

The D₂-like family members are Gi coupled receptors that inhibit adenylate cyclase and increase intracellular calcium levels. The D₂ receptor is the most abundant of the D₂-like receptors and is located in brain areas such as the striatum and substantia nigra, and in peripheral areas such as the heart, pituitary gland and kidney. The D₃ receptor is found abundantly in the islands of Calleja with distinct cluster populations in the ventral striatum/nucleus accumbens regions, olfactory tubercle, dendate gyrus and striatal cortex.

Expression of the D₄ receptor has been documented by in situ RNA hybridization and immunohistochemical studies. Recently, studies revealed that D₄ expression is highest in the entorhinal cortex, lateral septal nucleus, hippocampus and the medial preoptic area of the hypothalamus. Localization of D₄ is distinct from the distribution of D₂ in the brain, as D₂ receptors are most abundant in striatal areas. The expression of D₄ receptors in the MPOA of the hypothalamus is of importance to the facilitation of penile erection in view of the role of the hypothalamus as an area of integration between the cortex and the spinal pathways. The participation of D₄ receptors in other CNS regions, thalamic, subthalamic and spinal can not be excluded.

The present invention identifies a therapeutic use for acetamides and benzamides of formula (I) in the treatment of sexual dysfunction in mammals. More specifically, these compounds are useful in the treatment of sexual dysfunction including, but not limited to, male erectile dysfunction (MED).

### SUMMARY OF THE INVENTION

The present invention relates to a method of treating sexual dysfunction in a mammal, in particular humans, comprising administering to the mammal a therapeutically effective amount of a compound of formula (I)

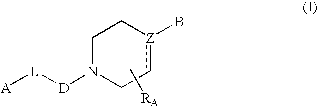

or a pharmaceutically acceptable salt, ester, amide, or prodrug thereof, wherein

A is selected from aryl, arylalkyl, cycloalkyl, cycloalkylalkyl, heterocycle, or heterocyclealkyl;

L is selected from the group consisting of-N(R₇)C(O)— and —C(O)N(R₇)— wherein the left end of said —N(R₇)C(O)— or —C(O)N(R₇)— is attached to A and the right end is attached to D;

D is selected from alkylene, fluoroalkylene, and hydroxyalkylene;

R_{A} is selected from hydrogen or alkyl;

Z is selected from N, C or CH;

— is a bond when Z is C and — is absent when Z is N or CH;

B is selected from

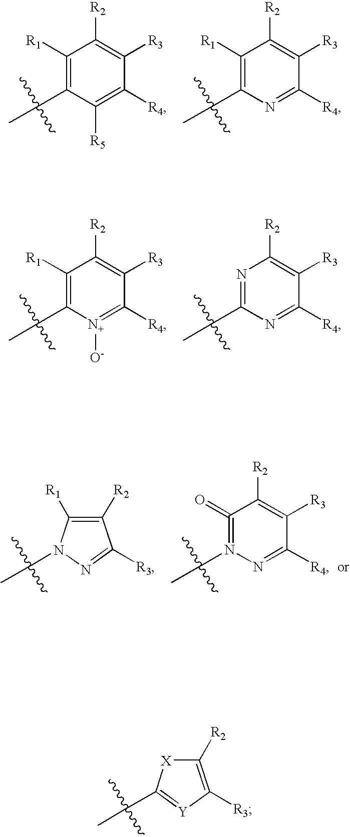

R₁, R₂, R₃, R₄ and R₅ are each independently selected from hydrogen, alkoxy, alkenyl, alkyl, alkylsulfinyl, alkylsulfonyl, alkylthio, alkynyl, alkoxycarbonyl, alkylcarbonyl, alkylcarbonyloxy, carboxy, cyano, formyl, halogen, haloalkoxy, haloalkyl, hydroxy, hydroxyalkyl, mercapto, nitro, —NZ₁Z₂, (NZ₃Z₄)carbonyl, or (NZ₃Z₄)sulfonyl;

Z₁ and Z₂ are each independently selected from hydrogen, alkyl, alkylcarbonyl, alkylsulfonyl, aryl, arylalkyl, arylalkylsulfonyl, arylsulfonyl, or formyl;

Z₃ and Z₄ are each independently selected from hydrogen, alkyl, aryl, or arylalkyl;

X is selected from N(R₆), O or S;

Y is selected from C(R₄) or N;

R₆ is selected from hydrogen or alkyl; and

R₇ is selected from hydrogen or alkyl.

### DETAILED DESCRIPTION OF THE INVENTION

All patents, patent applications, and literature references cited in the specification are herein incorporated by reference in their entirety.

In its principle embodiment, the present invention relates to a method of treating sexual dysfunction in a mammal, in particular humans, comprising administering to the mrnammal a therapeutically effective amount of a compound of formula (I)

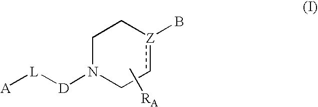

or a pharmaceutically acceptable salt, ester, amide, or prodrug thereof, wherein

A is selected from aryl, arylalkyl, cycloalkyl, cycloalkylalkyl, heterocycle, or heterocyclealkyl;

L is selected from the group consisting of —N(R₇)C(O)— and —C(O)N(R₇)— wherein the left end of said —N(R₇)C(O)— or —C(O)N(R₇)— is attached to A and the right end is attached to D;

D is selected from alkylene, fluoroalkylene, and hydroxyalkylene;

R_{A} is selected from hydrogen or alkyl;

Z is selected from N, C or CH;

— is a bond when Z is C and — is absent when Z is N or CH;

B is selected from

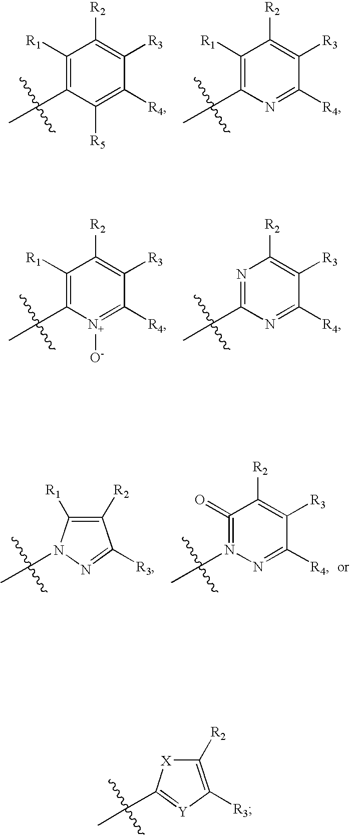

R₁, R₂, R₃, R₄ and R₅ are each independently selected from hydrogen, alkoxy, alkenyl, alkyl, alkylsulfinyl, alkylsulfonyl, alkylthio, alkynyl, alkoxycarbonyl, alkylcarbonyl, alkylcarbonyloxy, carboxy, cyano, formyl, halogen, haloalkoxy, haloalkyl, hydroxy, hydroxyalkyl, mercapto, nitro, —NZ₁Z₂, (NZ₃Z₄)carbonyl, or (NZ₃Z₄)sulfonyl;

Z₁ and Z₂ are each independently selected from hydrogen, alkyl, alkylcarbonyl, alkylsulfonyl, aryl, arylalkyl, arylalkylsulfonyl, arylsulfonyl, or formyl;

Z₃ and Z₄ are each independently selected from hydrogen, alkyl, aryl, or arylalkyl;

X is selected from N(R₆), O or S;

Y is selected from C(R₄) or N;

R₆ is selected from hydrogen or alkyl; and

R₇ is selected from hydrogen or alkyl.

In another embodiment, the present invention relates to a method of treating sexual dysfunction in a mammal comprising administering to the mammal a therapeutically effective amount of a compound of formula (I) wherein A is aryl; B is

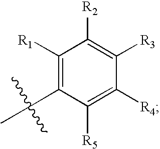

Z is N; — is absent; L is — N(R₇)C(O)—; and D, R₁, R₂, R₃, R₄, R₅, R₇, and R_{A} are as defined in formula (1).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

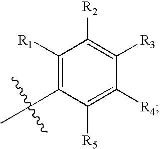

R₁ is selected from hydrogen, alkoxy, alkyl, cyano, halogen, or nitro; R₂ is selected from hydrogen, cyano, or halogen; R₃, R₄, and R₅ are hydrogen; Z is N; — is absent; D is —CH₂—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

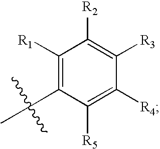

R₁ is selected from hydrogen, alkoxy, alkyl, cyano, halogen, or nitro; R₂ is selected from hydrogen, cyano, or halogen; R₃, R₄, and R₅ are hydrogen; Z is N; — is absent; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

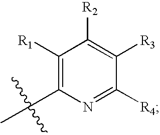

Z is N; — is absent; L is —N(R₇)C(O)—; and D, R₁, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

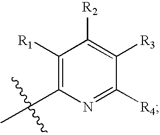

R₁ is selected from hydrogen, alkyl, cyano, or halogen; R₂, R₃, and R₄ are hydrogen; Z is N; — is absent; D is —CH₂—; L is —N(R₇)C(O)—; and R₇ and R_{A} are as defined in formula (D).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

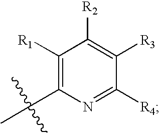

R₁ is selected from hydrogen, alkyl, cyano, or halogen; R₂, R₃, and R₄ are hydrogen; Z is N; — is absent; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is tetrahydronaphthalenyl substituted with 0, 1, 2, 3, 4, or 5 substituents selected from alkoxy, alkoxycarbonyl, alkyl, cyano, halogen, haloalkoxy, haloalkyl, or nitro; B is

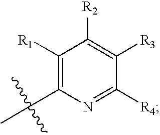

R₁ is selected from hydrogen, alkyl, cyano, or halogen; R₂, R₃, and R₄ are hydrogen; Z is N; — is absent; D is —CH₂—; L is —N(R₇)C(O)—; and R₇ and R_{A} are as defined in formual (I).

p In another embodiment, the present invention relates to a method of treating sexual dysfunction in a mammal comprising administering to the mammal a therapeutically effective amount of a compound of formula (I) wherein A is aryl wherein the aryl is tetrahydronaphthalenyl substituted with 0, 1, 2, 3, 4, or 5 substituents selected from alkoxy, alkoxycarbonyl, alkyl, cyano, halogen, haloalkoxy, haloalkyl, or nitro; B is

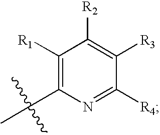

R₁ is selected from hydrogen, alkyl, cyano, or halogen; R₂, R₃, and R₄ are hydrogen; Z is N; — is absent; D is —CH(CH₃)—; L is —N(R₇)C(O)—; and R₇ and R_{A} are as defined in formual (I).

In another embodiment, the present invention relates to a method of treating sexual dysfunction in a mammal comprising administering to the mammal a therapeutically effective amount of a compound of formula (I) wherein A is aryl; B is

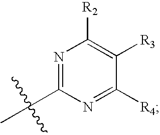

Z is N; is absent; L is —N(R₇)C(O)—; and D, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

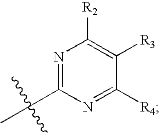

R₂, R₃, and R₄ are hydrogen; Z is N; — is absent; D is —CH₂—; L is —N(R₇)C(O)—; and R₇ and R_{A} are as defined in formula (I).

p In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

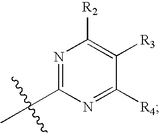

R₂, R₃, and R₄ are hydrogen; Z is N; — is absent; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (1) wherein A is aryl; B is

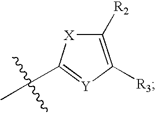

Z is N; — is absent; L is —N(R₇)C(O)—; and D, X, Y, R₂, R₃, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

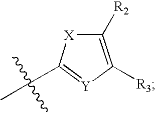

R₂ and R₃ are hydrogen; X is S; Y is N; Z is N; — is absent; D is —CH₂—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

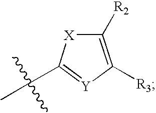

R₂ and R₃ are hydrogen; X is S; Y is N; Z is N; — is absent; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is heterocycle; B is

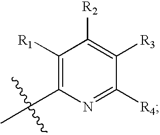

Z is N; — is absent; L is —N(R₇)C(O)—; and D, R₁, R₂, R₃, R₄, R₇ and R_{A} are as defined in formula (I).

In another embodiment, the present invention relates to a method of treating sexual dysfunction in a mammal comprising administering to the mammal a therapeutically effective amount of a compound of formula (I) wherein A is heterocycle wherein the heterocycle is selected from furyl, imidazolyl, 1,3-oxazolyl, pyrazinyl, pyridazinyl, pyridinyl, pyrimidinyl, pyrrolyl, 1,3-thiazolyl, or thienyl wherein the heterocycle is independently substituted with 0, 1, 2, or 3 substituents selected from alkoxy, alkoxycarbonyl, alkyl, cyano, halogen, haloalkoxy, haloalkyl, or nitro; B is

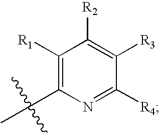

Z is N; — is absent; D is —CH₂—; L is —N(R₇)C(O)—; and R₁, R₂, R₃, R₄, R₇ and R_{A} are as defined in formula (I).

In another embodiment, the present invention relates to a method of treating sexual dysfunction in a mammal comprising administering to the mammal a therapeutically effective amount of a compound of formula (I) wherein A is heterocycle wherein the heterocycle is selected from furyl, imidazolyl, 1,3-oxazolyl, pyrazinyl, pyridazinyl, pyridinyl, pyrimidinyl, pyrrolyl, 1,3-thiazolyl, or thienyl wherein the heterocycle is independently substituted with 0, 1, 2, or 3 substituents selected from alkoxy, alkoxycarbonyl, alkyl, cyano, halogen, haloalkoxy, haloalkyl, or nitro; B is

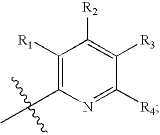

Z is N; — is absent; D is —CH(CH₃)—; L is —N(R₇)C(O)—; and R₁, R₂, R₃, R₄, R₇ and R_{A} are as defined in formula (I).

In another embodiment, the present invention relates to a method of treating sexual dysfunction in a mammal comprising administering to the mammal a therapeutically effective amount of a compound of formula (I) wherein A is heterocycle wherein the heterocycle is pyridinyl independently substituted with 0, 1, 2, or 3 substituents selected from alkoxy, alkoxycarbonyl, alkyl, cyano, halogen, haloalkoxy, haloalkyl, or nitro; B is

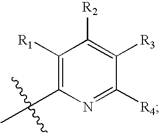

R₁ is selected from the group consisting of hydrogen, alkyl, cyano, or halogen; R₂, R₃, and R₄ are hydrogen; Z is N; — is absent; D is —CH₂—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is heterocycle wherein the heterocycle is pyridinyl independently substituted with 0, 1, 2, or 3 substituents selected from alkoxy, alkoxycarbonyl, alkyl, cyano, halogen, haloalkoxy, haloalkyl, or nitro; B is

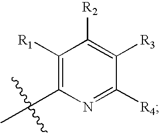

R₁ is selected from the group consisting of hydrogen, alkyl, cyano, or halogen; R₂, R₃, and R₄ are hydrogen; Z is N; — is absent; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl; B is

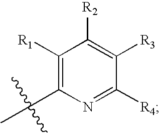

Z is N; — is absent; L is —N(R₇)C(O)—; and D, R₁, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

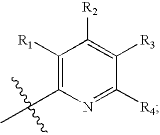

R₁ is selected from hydrogen, alkyl, cyano, or halogen; R₂, R₃, and R₄ are hydrogen; Z is N; — is absent; D is —CH₂—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

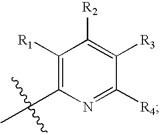

R₁ is selected from hydrogen, alkyl, cyano, or halogen; R₂, R₃, and R₄ are hydrogen; Z is N; — is absent; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is arylalkyl; B is

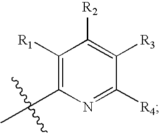

Z is N; — is absent; L is —N(R₇)C(O)—; and D, R₁, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is arylalkyl wherein the aryl of arylalkyl is phenyl substituted with 0, 1, 2, 3, 4, or 5 substituents selected from alkoxy, alkoxycarbonyl, alkyl, cyano, halogen, haloalkoxy, haloalkyl, or nitro; B is

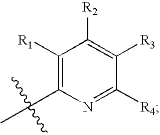

R₁ is selected from hydrogen, alkyl, cyano, or halogen; R₂, R₃, and R₄ are hydrogen; Z is N; — is absent; D is —CH₂—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is arylalkyl wherein the aryl of arylalkyl is phenyl substituted with 0, 1, 2, 3, 4, or 5 substituents selected from alkoxy, alkoxycarbonyl, alkyl, cyano, halogen, haloalkoxy, haloalkyl, or nitro; B is

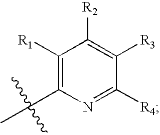

R₁ is selected from hydrogen, alkyl, cyano, or halogen; R₂, R₃, and R₄ are hydrogen; Z is N; — is absent; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

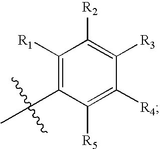

Z is CH; — is absent; L is —N(R₇)C(O)—; and D, R₁, R₂, R₃, R₄, R₅, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

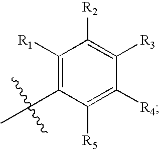

R₁ is selected from hydrogen, alkoxy, alkyl, or halogen; R₂ is selected from hydrogen or halogen; R₃, R₄, and R₅ are hydrogen; Z is CH; — is absent; D is —CH₂—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

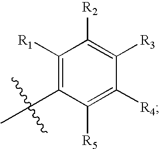

R₁ is selected from hydrogen, alkoxy, alkyl, or halogen; R₂ is selected from hydrogen or halogen; R₃, R₄, and R₅ are hydrogen; Z is CH; — is absent; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

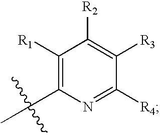

Z is CH; — is absent; L is —N(R₇)C(O)—; and D, R₁, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

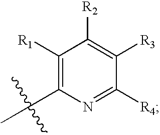

R₁ is selected from hydrogen, alkyl, cyano, or halogen; R₂, R₃, and R₄ are hydrogen; Z is CH; — is absent; D is —CH₂—; L is —N(R₇)C(O)—; and R₇ and R_{A} are as defined on formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

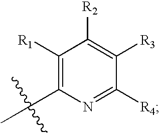

R₁ is selected from hydrogen, alkyl, cyano, or halogen; R₂, R₃, and R₄ are hydrogen; Z is CH; — is absent; D is —CH(CH₃)—; L is —N(R₇)C(O)—; and R₇ and R_{A} are as defined on formula (I).

In another embodiment, the present invention relates to a method of treating sexual dysfunction in a mammal comprising administering to the mammal a therapeutically effective amount of a compound of formula (I) wherein A is aryl; B is

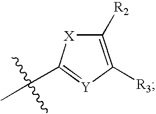

Z is CH; — is absent; L is —N(R₇)C(O)—; and D, R₂, R₃, R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

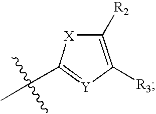

X is S; Y is N; R₂ and R₃ are hydrogen; Z is CH; — is absent; D is —CH₂—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

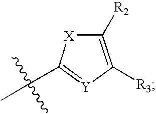

X is S; Y is N; R₂ and R₃ are hydrogen; Z is CH; — is absent; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

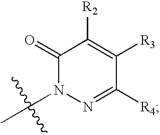

Z is CH; — is absent; L is —N(R₇)C(O)—; and D, R₂, R₃, R₄, R₇, and R_{A}.

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

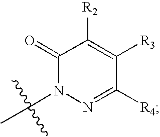

Z is CH; — is absent; D is —CH₂—; L is —N(R₇)C(O)—; R₂, R₃, and R₄ are hydrogen; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

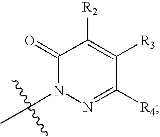

Z is CH; — is absent; D is —CH(CH₃)—; L is —N(R₇)C(O)—; R₂, R₃, and R₄ are hydrogen; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

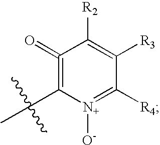

Z is CH; — is absent; L is —N(R₇)C(O)—; and D, R₁, R₂, R₃, R, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

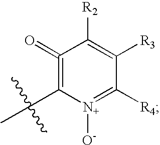

R₁, R₂, R₃, and R₄ are hydrogen; Z is CH; — is absent; D is —CH₂—; L is —N(R₇)C(O)—; and R₇ and R_{A} are as defined in formula (1).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

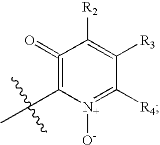

R₁, R₂, R₃, and R₄ are hydrogen; Z is CH; — is absent; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl; B is

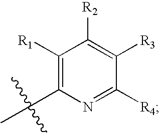

Z is CH; — is absent; L is —N(R₇)C(O)—; and D, R₁, R₂, R₃, R₄, R₇ and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

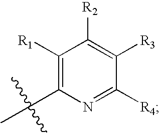

R₁ is selected from hydrogen, alkyl, cyano, or halogen; R₂, R₃, and R₄ are hydrogen; Z is CH; — is absent; D is —CH₂—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

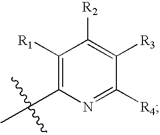

R₁ is selected from hydrogen, alkyl, cyano, or halogen; R₂, R₃, and R₄ are hydrogen; Z is CH; — is absent; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

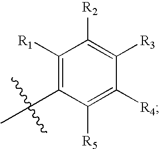

Z is C; — is a bond; L is —N(R₇)C(O)—; and D, R₁, R₂, R₃, R₄, R₅, R₇, and R_{A} are as defined on formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

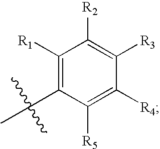

R₁ is selected from hydrogen, alkoxy, alkyl, cyano, halogen, or nitro; R₂ is selected from hydrogen, cyano, or halogen; R₃, R₄, and R₅ are hydrogen; Z is C; — is a bond; D is —CH₂—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

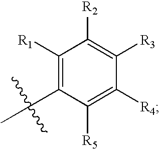

R₁ is selected from hydrogen, alkoxy, alkyl, cyano, halogen, or nitro; R₂ is selected from hydrogen, cyano, or halogen; R₃, R₄, and R₅ are hydrogen; Z is C; — is a bond; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

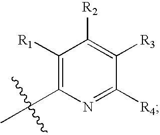

Z is C; — is a bond; L is —N(R₇)C(O)—; and D, R₁, R₂,R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

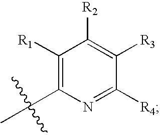

R₁ is selected from hydrogen, alkyl, cyano, or halogen; R₂, R₃, and R₄ are hydrogen; Z is C; — is a bond; D is —CH₂—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

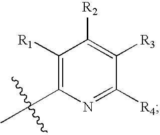

R₁ is selected from hydrogen, alkyl, cyano, or halogen; R₂, R₃, and R₄ are hydrogen; Z is C; — is a bond; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

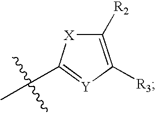

Z is C; — is a bond; L is —N(R₇)C(O)—; and D, X, Y, R₂, R₃, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

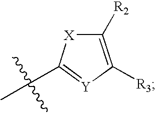

X is S; Y is C(R₄); R₂ and R₃ are hydrogen; R₄ is selected from hydrogen or cyano; Z is C; — is a bond; D is —CH₂—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

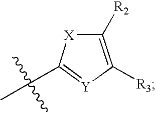

X is S; Y is C(R₄); R₂ and R₃ are hydrogen; R₄ is selected from hydrogen or cyano; Z is C; — is a bond; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl; B is

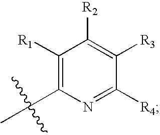

Z is C; — is a bond; L is —N(R₇)C(O)—; and D, R₁, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

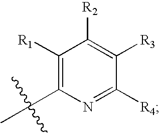

R₁ is selected from hydrogen, alkyl, cyano, or halogen; R₂, R₃, and R₄ are hydrogen; Z is C; — is a bond; D is —CH₂—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

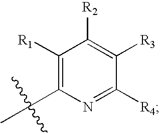

R₁ is selected from hydrogen, alkyl, cyano, or halogen; R₂, R₃, and R₄ are hydrogen; Z is C; — is a bond; D is —CH(CH₃)—; L is —N(R₇)C(O)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

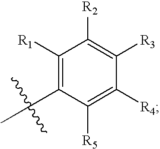

Z is N; — is absent; L is —C(O)N(R₇)—; and D, R₁, R₂, R₃, R₄, R₅, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

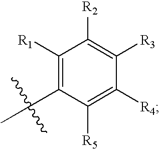

R₁ is selected from hydrogen, alkoxy, alkyl, cyano, halogen, or nitro; R₂ is selected from hydrogen, cyano, or halogen; R₃, R₄, and R₅ are hydrogen; Z is N; — is absent; D is —CH₂—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

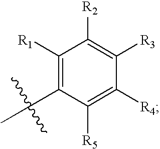

R₁ is selected from hydrogen, alkoxy, alkyl, cyano, halogen, or nitro; R₂ is selected from hydrogen, cyano, or halogen; R₃, R₄, and R₅ are hydrogen; Z is N; — is absent; D is —CH(CH₃)—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

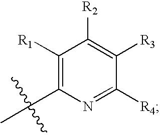

Z is N; — is absent; L is —C(O)N(R₇)—; and D, R₁, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

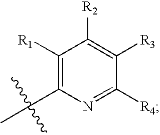

R₁ is selected from hydrogen, alkyl, cyano, or halogen; R₂, R₃, and R₄ are hydrogen; Z is N; — is absent; D is —CH₂—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

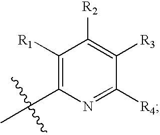

R₁ is selected from hydrogen, alkyl, cyano, or halogen; R₂, R₃, and R₄ are hydrogen; Z is N; — is absent; D is —CH(CH₃)—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

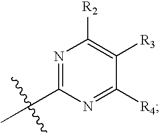

Z is N; — is absent; L is —C(O)N(R₇)—; and D, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

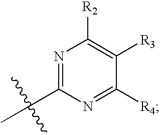

R₂, R₃, and R₄ are hydrogen; Z is N; — is absent; D is —CH₂—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

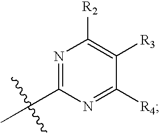

R₂, R₃, and R₄ are hydrogen; Z is N; — is absent; D is —CH(CH₃)—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl; B is

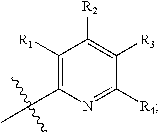

Z is N; — is absent; L is —C(O)N(R₇)—; and D, R₁, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

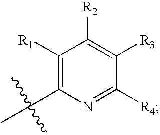

R₁ is selected from hydrogen, alkyl, cyano, or halogen; R₂, R₃, and R₄ are hydrogen; Z is N; — is absent; D is —CH₂—; L is —C(O)N(R₇)—; and R₇ and R_{A} are as defined as in formula (I).

In another embodiment, the present invention relates to a method of treating sexual dysfunction in a mammal comprising administering to the mammal a therapeutically effective amount of a compound of formula (I) wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

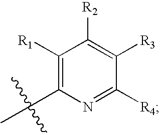

R₁ is selected from hydrogen, alkyl, cyano, or halogen; R₂, R₃, and R₄ are hydrogen; Z is N; — is absent; D is —CH(CH₃)—; L is —C(O)N(R₇)—; and R₇ and R_{A} are as defined as in formula (I).

In another embodiment, the present invention relates to a method of treating sexual dysfunction in a mammal comprising administering to the mammal a therapeutically effective amount of a compound of formula (I) wherein A is aryl; B is

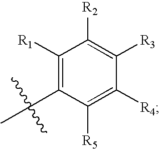

Z is CH; — is absent; L is —C(O)N(R₇)—; and d, R₁, R₂, R₃, R₄, R₅, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

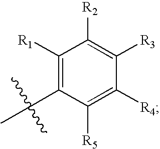

R₁ is selected from hydrogen, alkoxy, alkyl, cyano, halogen, or nitro; R₂ is selected from hydrogen, cyano, or halogen; R₃, R₄, and R₅ are hydrogen; Z is CH; — is absent; D is —CH₂—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

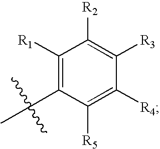

R₁ is selected from hydrogen, alkoxy, alkyl, cyano, halogen, or nitro; R₂ is selected from hydrogen, cyano, or halogen; R₃, R₄, and R₅ are hydrogen; Z is CH; — is absent; D is —CH(CH₃)—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

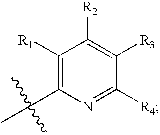

Z is CH; — is absent; L is —C(O)N(R₇)—; and D, R₁, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

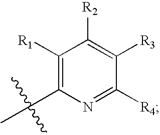

R₁ is selected from hydrogen, alkyl, cyano, or halogen; R₂, R₃, and R₄ are hydrogen; Z is CH; — is absent; D is —CH₂—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

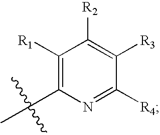

R₁ is selected from hydrogen, alkyl, cyano, or halogen; R₂, R₃, and R₄ are hydrogen; Z is CH; — is absent; D is —CH(CH₃)—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

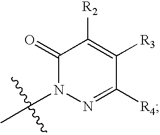

Z is CH; — is absent; L is —C(O)N(R₇)—; and D, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

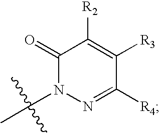

R₂, R₃, and R₄ are hydrogen; Z is CH; — is absent; D is —CH₂—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

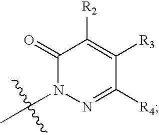

R₂, R₃, and R₄ are hydrogen; Z is CH; — is absent; D is —CH(CH₃)—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

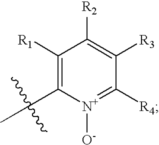

Z is CH; — is absent; L is —C(O)N(R₇)—; and D, R₁, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

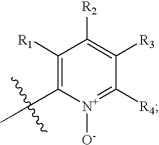

R₁, R₂, R₃, and R₄ are hydrogen; Z is CH; — is absent; D is —CH₂—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

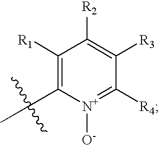

R₁, R₂, R₃, and R₄ are hydrogen; Z is CH; — is absent; D is —CH(CH₃)—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl; B is

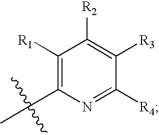

Z is CH; — is absent; L is —C(O)N(R₇)—; and D, R₁, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

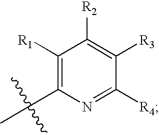

R₁ is selected from hydrogen, alkyl, cyano, or halogen; R₂, R₃, and R₄ are hydrogen; Z is CH; — is absent; D is —CH₂—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

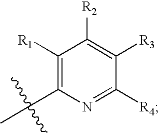

R₁ is selected from hydrogen, alkyl, cyano, or halogen; R₂, R₃, and R₄ are hydrogen; Z is CH; — is absent; D is —CH(CH₃)—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

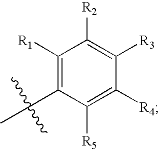

Z is C; is a bond; L is —C(O)N(R₇)—; and D, R₁, R₂, R₃, R₄, R₅, R₇, and R_{A} are as defined in formula (1).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

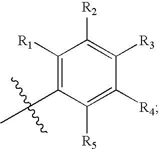

R₁ is selected from hydrogen, alkoxy, alkyl, cyano, halogen, or nitro; R₂ is selected from hydrogen, cyano, or halogen; R₃, R₄, and R₅ are hydrogen; Z is C; — is a bond; D is —CH₂—; L is —C(O)N(R₇)—; and R₇ and R_{A} are as defined in formula (D.

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

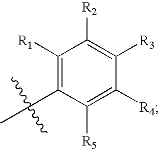

R₁ is selected from hydrogen, alkoxy, alkyl, cyano, halogen, or nitro; R₂ is selected from hydrogen, cyano, or halogen; R₃, R₄, and R₅ are hydrogen; Z is C; — is a bond; D is —CH(CH₃)—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

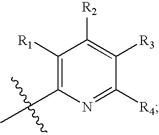

Z is C; — is a bond; L is —C(O)N(R₇)—; and D, R₁, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

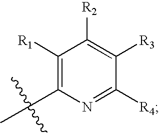

R₁ is selected from hydrogen, alkyl, cyano, or halogen; R₂, R₃, and R₄ are hydrogen; Z is C; — is a bond; D is —CH₂—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

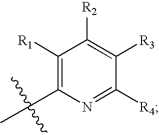

R₁ is selected from hydrogen, alkyl, cyano, or halogen; R₂, R₃, and R₄ are hydrogen; Z is C; — is a bond; D is —CH(CH₃)—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is naphthyl substituted with 0, 1, 2, 3, 4, or 5 substituents selected from alkoxy, alkoxycarbonyl, alkyl, cyano, halogen, haloalkoxy, haloalkyl, or nitro; B is

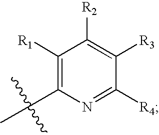

R₁ is selected from hydrogen, alkyl, cyano, or halogen; R₂, R₃, and R₄ are hydrogen; Z is C; — is a bond; D is —CH₂—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is naphthyl substituted with 0, 1, 2, 3, 4, or 5 substituents selected from alkoxy, alkoxycarbonyl, alkyl, cyano, halogen, haloalkoxy, haloalkyl, or nitro; B is

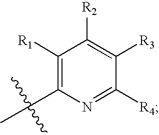

R₁ is selected from hydrogen, alkyl, cyano, or halogen; R₂, R₃, and R₄ are hydrogen; Z is C; — is a bond; D is —CH(CH₃)—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl; B is

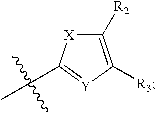

Z is C; — is a bond; L is —C(O)N(R₇)—; and D, R₂, R₃, R₇, and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

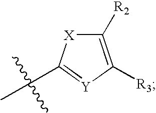

R₂ and R₃ are hydrogen; X is S; Y is N; Z is C; — is a bond; D is —CH₂—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

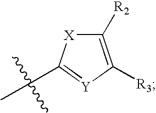

R₂ and R₃ are hydrogen; X is S; Y is N; Z is C; — is a bond; D is —CH(CH₃)—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

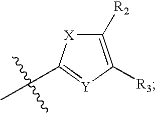

R₂ and R₃ are hydrogen; X is 0; Y is N; Z is C; — is a bond; D is —CH₂—; L is —C(O)N(R₇)—; are R₇ and R_{A} are as defined in formula (I.

In another embodiment, the present invention relates to a method of treating sexual dysfunction in a mammal comprising administering to the mammal a therapeutically effective amount of a compound of formula (I) wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

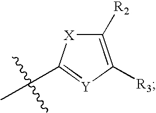

R₂ and R₃ are hydrogen; X is O; Y is N; Z is C; — is a bond; D is —CH(CH₃)—; L is —C(O)N(R₇)—; are R₇ and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl; B is

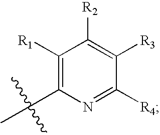

Z is C; — is a bond; L is —C(O)N(R₇)—; and D, R₁, R₂, R₃, R₄, R₇, and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

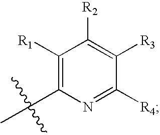

R₁ is selected from hydrogen, alkyl, cyano, or halogen; R₂, R₃, and R₄ are hydrogen; Z is C; — is a bond; D is —CH₂—; L is —C(O)N(R₇)—; and R₇ and R_{A} are as defined in formula (I).

In another embodiment, the present invention relates to a method of treating sexual dysfunction in a mammal comprising administering to the mammal a therapeutically effective amount of a compound of formula (I) wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

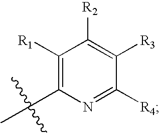

R₁ is selected from hydrogen, alkyl, cyano, or halogen; R₂, R₃, and R₄ are hydrogen; Z is C; — is a bond; D is —CH(CH₃)—; L is —C(O)N(R₇)—; and R₇ and R_{A} are as defined in formula (I).

In another embodiment of the present invention, compounds of formula (II)

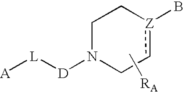

or a pharmaceutically acceptable salt, ester, amide, or prodrug thereof, are disclosed wherein

A is selected from aryl, arylalkyl, cycloalkyl, or cycloalkylalkyl;

L is selected from —N(R₇)C(O)— or —C(O)N(R₇)— wherein the left end of the —N(R₇)C(O)— and —C(O)N(R₇)— is attached to A and the right end is attached to D;

D is selected from the group consisting of alkylene, fluoroalkylene, and hydroxyalkylene;

R_{A} is selected from hydrogen or alkyl;

Z is selected from N, C or CH;

— is a bond when Z is C and — is absent when Z is N or CH;

B is selected from

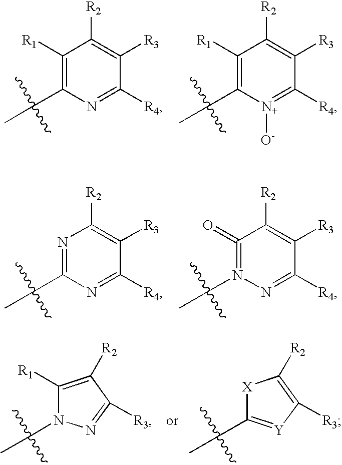

R₁, R₂, R₃, and R₄ are each independently selected from hydrogen, alkoxy, alkenyl, alkyl, alkylsulfinyl, alkylsulfonyl, alkylthio, alkynyl, alkoxycarbonyl, alkylcarbonyl, alkylcarbonyloxy, carboxy, cyano, formyl, halogen, haloalkoxy, haloalkyl, hydroxy, hydroxyalkyl, mercapto, nitro, —NZ₁Z₂, (NZ₃Z₄)carbonyl, or (NZ₃Z₄)sulfonyl;

Z₁ and Z₂ are each independently selected from hydrogen, alkyl, alkylcarbonyl, alkylsulfonyl, aryl, arylalkyl, arylalkylsulfonyl, arylsulfonyl, or formyl;

Z₃ and Z₄ are each independently selected from hydrogen, alkyl, aryl, or arylalkyl;

X is selected from N(R₆), O or S;

Y is selected from C(R₄) or N;

R₆ is selected from hydrogen or alkyl; and

R₇ is selected from hydrogen or alkyl.

In another embodiment of the present invention, compounds of formula (II) are disclosed wherein A is aryl; B is

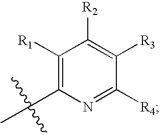

Z is N; — is absent; L is —N(R₇)C(O)—; and D, R₁, R₂, R₃, R₄, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

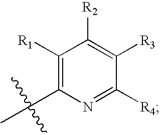

R₁ is selected from hydrogen, alkyl, cyano, or halogen; R₂, R₃, and R₄ are hydrogen; Z is N; — is absent; D is —CH₂—; L is —N(R₇)C(O)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

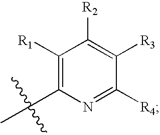

R₁ is selected from hydrogen, alkyl, cyano, or halogen; R₂, R₃, and R₄ are hydrogen; Z is N; is absent; D is —CH(CH₃)—; L is —N(R₇)C(O)—; and R₇ and R_{A} are as defined in formula (II).

In another embodiment of the present invention, compounds of formula (II) are disclosed wherein A is aryl wherein the aryl is tetrahydronaphthalenyl substituted with 0, 1, 2, 3, 4, or 5 substituents selected from alkoxy, alkoxycarbonyl, alkyl, cyano, halogen, haloalkoxy, haloalkyl, or nitro; B is

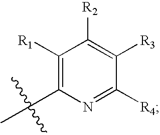

R₁ is selected from hydrogen, alkyl, cyano, or halogen; R₂, R₃, and R₄ are hydrogen; Z is N; — is absent; D is —CH₂—; L is —N(R₇)C(O)—; and R₇ and R_{A} are as defined in formula (II).

In another embodiment of the present invention, compounds of formula (II) are disclosed wherein A is aryl wherein the aryl is tetrahydronaphthalenyl substituted with 0, 1, 2, 3, 4, or 5 substituents selected from alkoxy, alkoxycarbonyl, alkyl, cyano, halogen, haloalkoxy, haloalkyl, or nitro; B is

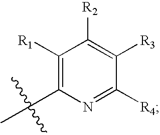

R₁ is selected from hydrogen, alkyl, cyano, or halogen; R₂, R₃, and R₄ are hydrogen; Z is N; — is absent; D is —CH(CH₃)—; L is —N(R₇)C(O)—; and R₇ and R_{A} are as defined in formula (II).

In another embodiment of the present invention, compounds of formula (II) are disclosed wherein A is aryl; B is

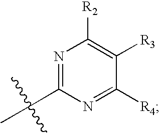

Z is N; — is absent; L is —N(R₇)C(O)—; and D, R₂, R₃, R₄, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

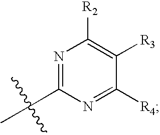

R₂, R₃, and R₄ are hydrogen; Z is N; — is absent; D is —CH₂—; L is —N(R₇)C(O)—; R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

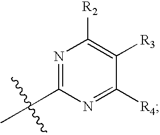

R₂, R₃, and R₄ are hydrogen; Z is N; — is absent; D is —CH(CH₃)—; L is —N(R₇)C(O)—; R₇ and R_{A} are as defined in formula (II).

In another embodiment of the present invention, compounds of formula (II) are disclosed wherein A is aryl; B is

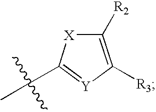

Z is N; — is absent; L is —N(R₇)C(O)—; and D, X, Y, R₂, R₃, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

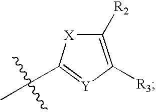

R₂ and R₃ are hydrogen; X is S; Y is N; Z is N; — is absent; D is —CH₂—; L is —N(R₇)C(O)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

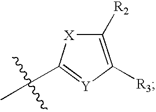

R₂ and R₃ are hydrogen; X is S; Y is N; Z is N; — is absent; D is —CH(CH₃)—; L is —N(R₇)C(O)—; and R₇ and R_{A} are as defined in formula (II).

In another embodiment of the present invention, compounds of formula (II) are disclosed wherein A is cycloalkyl; B is

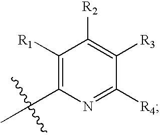

Z is N; — is absent; L is —N(R₇)C(O)—; and D, R₁, R₂, R₃, R₄, R₇, and R_{A} are as defined in formula (II).

In another embodiment of the present invention, compounds of formula (II) are disclosed wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

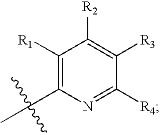

R₁ is selected from hydrogen, alkyl, cyano, or halogen; R₂, R₃, and R₄ are hydrogen; Z is N; — is absent; D is —CH₂—; L is —N(R₇)C(O)—; and R₇ and R_{A} are as defined in formula (II).

In another embodiment of the present invention, compounds of formula (II) are disclosed wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

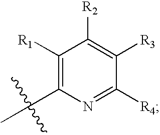

R₁ is selected from hydrogen, alkyl, cyano, or halogen; R₂, R₃, and R are hydrogen; Z is N; — is absent; D is —CH(CH₃)—; L is —N(R₇)C(O)—; and R₇ and R_{A} are as defined in formula (II).

In another embodiment of the present invention, compounds of formula (II) are disclosed wherein A is arylalkyl; B is

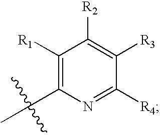

Z is N; — is absent; L is —N(R₇)C(O)—; and D, R₁, R₂, R₃, R₄, R₇, and R_{A} are as defined in formula (II).

In another embodiment of the present invention, compounds of formula (II) are disclosed wherein A is arylalkyl wherein the aryl of arylalkyl is phenyl substituted with 0, 1, 2, 3, 4, or 5 substituents selected from alkoxy, alkoxycarbonyl, alkyl, cyano, halogen, haloalkoxy, haloalkyl, or nitro; B is

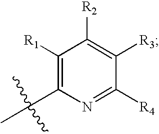

R₁ is selected from hydrogen, alkyl, cyano, or halogen; R₂, R₃, and R₄ are hydrogen; Z is N; — is absent; D is —CH₂—; L is —N(R₇)C(O)—; and R₇ and R_{A} are as defined in formula (II).

In another embodiment of the present invention, compounds of formula (II) are disclosed wherein A is arylalkyl wherein the aryl of arylalkyl is phenyl substituted with 0, 1, 2, 3, 4, or 5 substituents selected from alkoxy, alkoxycarbonyl, alkyl, cyano, halogen, haloalkoxy, haloalkyl, or nitro; B is

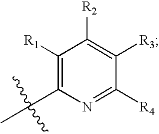

R₁ is selected from hydrogen, alkyl, cyano, or halogen; R₂, R₃, and R₄ are hydrogen; Z is N; — is absent; D is —CH(CH₃)—; L is —N(R₇)C(O)—; and R₇ and R_{A} are as defined in formula (II).

In another embodiment of the present invention, compounds of formula (II) are disclosed wherein A is aryl; B is

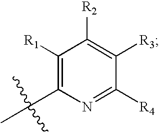

Z is CH; is absent; L is —N(R₇)C(O)—; and D, R₁, R₂, R₃, R₄, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

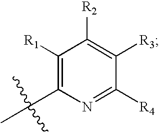

R₁ is selected from hydrogen, alkyl, cyano, or halogen; R₂, R₃, and R₄ are hydrogen; Z is CH; — is absent; D is —CH₂—; L is —N(R₇)C(O)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

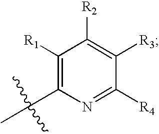

R₁ is selected from hydrogen, alkyl, cyano, or halogen; R₂, R₃, and R₄ are hydrogen; Z is CH; — is absent; D is —CH(CH₃)—; L is —N(R₇)C(O)—; and R₇ and R_{A} are as defined in formula (II).

In another embodiment of the present invention, compounds of formula (II) are disclosed wherein A is aryl; B is

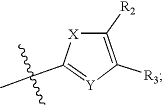

Z is CH; — is absent; L is —N(R₇)C(O)—; and D, X, Y, R₂, R₃, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

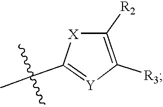

X is S; Y is N; R₂ and R₃ are hydrogen; Z is CH; — is absent; D is —CH₂—; L is —N(R₇)C(O)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

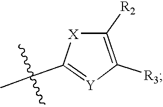

X is S; Y is N; R₂ and R₃ are hydrogen; Z is CH; — is absent; D is —CH(CH₃)—; L is —N(R₇)C(O)—; and R₇ and R_{A} are as defined in formula (II).

In another embodiment of the present invention, compounds of formula (II) are disclosed wherein A is aryl; B is

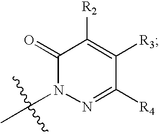

Z is CH; — is absent; L is —N(R₇)C(O)—; and D, R₂, R₃, R₄, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

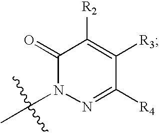

Z is CH; — is absent; D is —CH₂—; L is —N(R₇)C(O)—; R₂, R₃, and R₄ are hydrogen; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

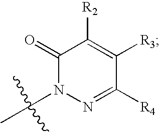

Z is CH; — is absent; D is —CH(CH₃)—; L is —N(R₇)C(O)—; R₂, R₃, and R₄ are hydrogen; and R₇ and R_{A} are as defined in formula (II).

In another embodiment of the present invention, compounds of formula (II) are disclosed wherein A is aryl; B is

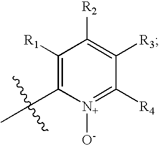

Z is CH; — is absent; L is —N(R₇)C(O)—; and D, R₁, R₂, R₃, R₄, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

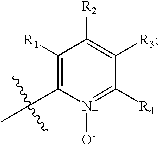

R₁, R₂, R₃, and R₄ are hydrogen; Z is CH; — is absent; D is —CH₂—; L is —N(R₇)C(O)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

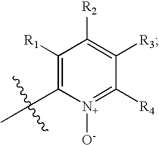

R₁, R₂, R₃, and R₄ are hydrogen; Z is CH; — is absent; D is —CH(CH₃)—; L is —N(R₇)C(O)—; and R₇ and R_{A} are as defined in formula (II).

In another embodiment of the present invention, compounds of formula (II) are disclosed wherein A is cycloalkyl; B is

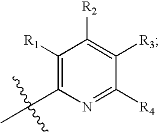

Z is CH; — is absent; L is —N(R₇)C(O)—; and D, R₁, R₂, R₃, R₄, R₇, and R_{A} are as defined in formula (II).

In another embodiment of the present invention, compounds of formula (II) are disclosed wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

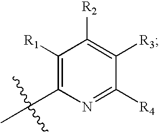

R₁ is selected from hydrogen, alkyl, cyano, or halogen; R₂, R₃, and R₄ are hydrogen; Z is CH; is absent; D is —CH₂—; L is —N(R₇)C(O)—; and R₇ and R_{A} are as defined in formula (II).

In another embodiment of the present invention, compounds of formula (II) are disclosed wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

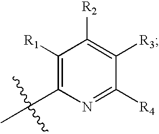

R₁ is selected from hydrogen, alkyl, cyano, or halogen; R₂, R₃, and R₄ are hydrogen; Z is CH; — is absent; D is —CH(CH₃)—; L is —N(R₇)C(O)—; and R₇ and R_{A} are as defined in formula (II).

In another embodiment of the present invention, compounds of formula (II) are disclosed wherein A is aryl; B is

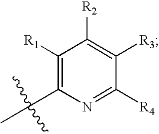

Z is C; — is a bond; L is —N(R₇)C(O)—; and D, R₁, R₂, R₃, R₄, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

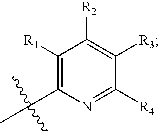

R₁ is selected from hydrogen, alkyl, cyano, or halogen; R₂, R₃, and R₄ are hydrogen; Z is C; — is a bond; D is —CH₂—; L is —N(R₇)C(O)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

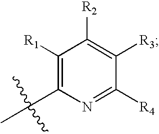

R₁ is selected from hydrogen, alkyl, cyano, or halogen; R₂, R₃, and R₄ are hydrogen; Z is C; — is a bond; D is —CH(CH₃)—; L is —N(R₇)C(O)—; and R₇ and R_{A} are as defined in formula (II).

In another embodiment of the present invention, compounds of formula (II) are disclosed wherein A is aryl; B is

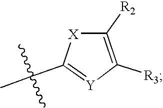

Z is C; — is a bond; L is —N(R₇)C(O)—; and D, X, Y, R₂, R₃, R₇, and R_{A} are as defined informula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

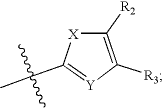

X is S; Y is C(h); R₂ and R₃ are hydrogen; R₄ is selected from hydrogen or cyano; Z is C; — is a bond; D is —CH₂—; L is —N(R₇)C(O)—; and R₇ and R_{A} are as defined in formula (It).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

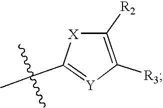

X is S; Y is C(R₄); R₂ and R₃ are hydrogen; R₄ is selected from hydrogen or cyano; Z is C; is a bond; D is —CH(CH₃)—; L is —N(R₇)C(O)—; and R₇ and R_{A} are as defined in formula (II).

In another embodiment of the present invention, compounds of formula (II) are disclosed wherein A is cycloalkyl; B is

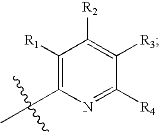

Z is C; — is a bond; L is —N(R₇)C(O)—; and D, R₁, R₂, R₃, R₄, R₇, and R_{A} are as defined in formula (II).

In another embodiment of the present invention, compounds of formula (II) are disclosed wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

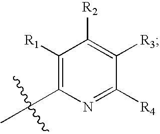

R₁ is selected from hydrogen, alkyl, cyano, or halogen; R₂, R₃, and R₄ are hydrogen; Z is C; — is a bond; D is —CH₂—; L is —N(R₇)C(O)—; and R₇ and R_{A} are as defined in formula (II).

In another embodiment of the present invention, compounds of formula (II) are disclosed wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

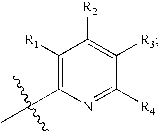

R₁ is selected from hydrogen, alkyl, cyano, or halogen; R₂, R₃, and R₄ are hydrogen; Z is C; — is a bond; D is —CH(CH₃)—; L is —NR₇)C(O)—; and R₇ and R_{A} are as defined in formula (II).

In another embodiment of the present invention, compounds of formula (II) are disclosed wherein A is aryl; B is

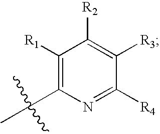

Z is N; — is absent; L is —C(O)N(R₇)—; and D, R₁, R₂, R₃, R₄, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

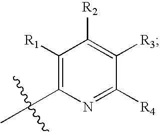

R₁ is selected from hydrogen, alkyl, cyano, or halogen; R₂, R₃, and R₄ are hydrogen; Z is N; — is absent; D is —CH₂—; L is —C(O)N(R₇)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

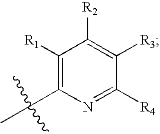

R₁ is selected from hydrogen, alkyl, cyano, or halogen; R₂, R₃, and R₄ are hydrogen; Z is N; — is absent; D is —CH(CH₃)—; L is —C(O)N(R₇)—; and R₇ and R_{A} are as defined in formula (II).

In another embodiment of the present invention, compounds of formula (II) are disclosed wherein A is aryl; B is

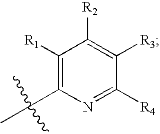

Z is N; — is absent; L is —C(O)N(R₇)—; and D, R₂, R₃, R₄, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

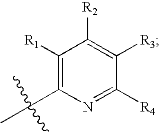

R₂, R₃, and R₄ are hydrogen; Z is N; — is absent; D is —CH₂—; L is —C(O)N(R₇)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

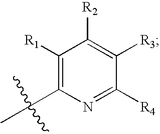

R₂,R₃, and R₄ are hydrogen; Z is N; — is absent; D is —CH(CH₃)—; L is —C(O)N(R₇)—; and R₇ and R_{A} are as defined in formula (II).

In another embodiment of the present invention, compounds of formula (II) are disclosed wherein A is cycloalkyl; B is

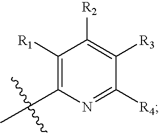

Z is N; — is absent; L is —C(O)N(R₇)—; and D, R₁, R₂, R₃, R₄, R₇, and R_{A} are as defined in formula (II).

In another embodiment of the present invention, compounds of formula (II) are disclosed wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

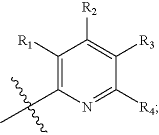

R₁ is selected from hydrogen, alkyl, cyano, or halogen; R₂, R₃, and R₄ are hydrogen; Z is N; — is absent; D is —CH₂—; L is —C(O)N(R₇)—; and R₇ and R_{A} are as defined in formula (II).

In another embodiment of the present invention, compounds of formula (II) are disclosed wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

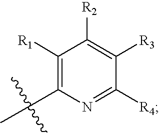

R₁ is selected from hydrogen, alkyl, cyano, or halogen; R₂, R₃, and R₄ are hydrogen; Z is N; — is absent; D is —CH(CH₃)—; L is —C(O)N(R₇)—; and R₇ and R_{A} are as defined in formula (II).

In another embodiment of the present invention, compounds of formula (II) are disclosed wherein A is aryl; B is

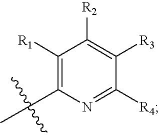

Z is CH; — is absent; L is —C(O)N(R₇)—; and D, R₁, R₂, R₃, R₄, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

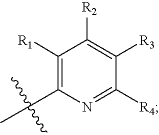

R₁ is selected from hydrogen, alkyl, cyano, or halogen; R₂, R₃, and R₄ are hydrogen; Z is CH; — is absent; D is —CH₂—; L is —C(O)N(R₇)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

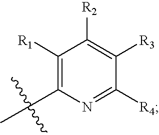

R₁ is selected from hydrogen, alkyl, cyano, or halogen; R₂, R₃, and R₄ are hydrogen; Z is CH; — is absent; D is —CH(CH₃)—; L is —C(O)N(R₇)—; and R₇ and R_{A} are as defined in formula (II).

In another embodiment of the present invention, compounds of formula (II) are disclosed wherein A is aryl; B is

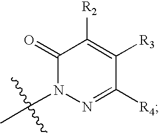

Z is CH; — is absent; L is —C(O)N(R₇)—; and D, R₂, R₃, R₄, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

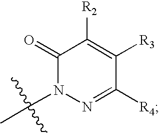

R₂, R₃, and R₄ are hydrogen; Z is CH; — is absent; D is —CH₂—; L is —C(O)N(R₇)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

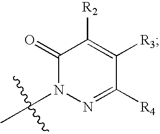

R₂, R₃, and R are hydrogen; Z is CH; — is absent; D is —CH(CH₃)—; L is —C(O)N(R₇)—; and R₇ and R_{A} are as defined in formula (II).

In another embodiment of the present invention, compounds of formula (II) are disclosed wherein A is aryl; B is

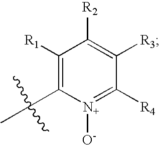

Z is CH; — is absent; L is —C(O)N(R₇)—; and D, R₁, R₂, R₃, R, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

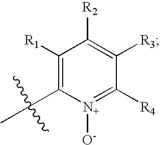

R₁, R₂, R₃, and R₄ are hydrogen; Z is CH; — is absent; D is —CH₂—; L is —C(O)N(R₇)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

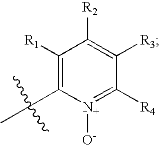

R₁, R₂, R₃, and R₄ are hydrogen; Z is CH; — is absent; D is —CH(CH₃)—; L is —C(O)N(R₇)—; and R₇ and R_{A} are as defined in formula (II).

In another embodiment of the present invention, compounds of formula (II) are disclosed wherein A is cycloalkyl; B is

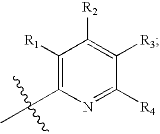

Z is CH; — is absent; L is —C(O)N(R₇)—; and D, R₁, R₂, R₃, R₄, R₇, and R_{A} are as defined in formula (II).

In another embodiment of the present invention, compounds of formula (II) are disclosed wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

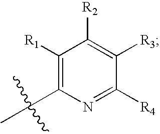

R₁ is selected from hydrogen, alkyl, cyano, or halogen; R₂, R₃, and R₄ are hydrogen; Z is CH; — is absent; D is —CH₂—; L is —C(O)N(R₇)—; and R₇ and R_{A} are as defined in formula (II).

In another embodiment of the present invention, compounds of formula (II) are disclosed wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

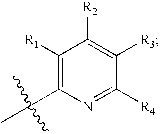

R₁ is selected from hydrogen, alkyl, cyano, or halogen; R₂, R₃, and R₄ are hydrogen; Z is CH; — is absent; D is —CH(CH₃)—; L is —C(O)N(R₇)—; and R₇ and R_{A} are as defined in formula (II).

In another embodiment of the present invention, compounds of formula (II) are disclosed wherein A is aryl; B is

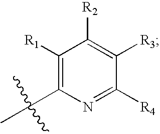

Z is C; — is a bond; L is —C(O)N(R₇)—; and D, R₁, R₂, R₃, R₄, R₇, and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

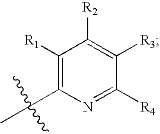

R₁ is selected from hydrogen, alkyl, cyano, or halogen; R₂, R₃, and R₄ are hydrogen; Z is C; — is a bond; D is —CH₂—; L is —C(O)N(R₇)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

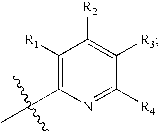

R₁ is selected from hydrogen, alkyl, cyano, or halogen; R₂, R₃, and R are hydrogen; Z is C; — is a bond; D is —CH(CH₃)—; L is —C(O)N(R₇)—; and R₇ and R_{A} are as defined in formula (II).

In another embodiment of the present invention, compounds of formula (II) are disclosed wherein A is aryl wherein the aryl is naphthyl substituted with 0, 1, 2, 3, 4, or 5 substituents selected from alkoxy, alkoxycarbonyl, alkyl, cyano, halogen, haloalkoxy, haloalkyl, or nitro; B is

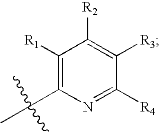

R₁ is selected from hydrogen, alkyl, cyano, or halogen; R₂, R₃, and R₄ are hydrogen; Z is C; — is a bond; D is —CH₂—; L is —C(O)N(R₇)—; and R₇ and R_{A} are as defined in formula (II).

In another embodiment of the present invention, compounds of formula (II) are disclosed wherein A is aryl wherein the aryl is naphthyl substituted with 0, 1, 2, 3, 4, or 5 substituents selected from alkoxy, alkoxycarbonyl, alkyl, cyano, halogen, haloalkoxy, haloalkyl, or nitro; B is

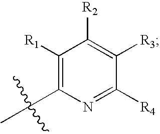

R₁ is selected from hydrogen, alkyl, cyano, or halogen; R₂, R₃, and R₄ are hydrogen; Z is C; — is a bond; D is —CH(CH₃)—; L is —C(O)N(R₇)—; and R₇ and R_{A} are as defined in formula (II).

In another embodiment of the present invention, compounds of formula (II) are disclosed wherein A is aryl; B is

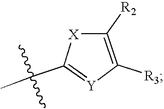

Z is C; — is a bond; L is —C(O)N(R₇)—; and D, X, Y, R₂, R₃, R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

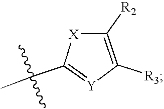

R₂ and R₃ are hydrogen; X is S; Y is N; Z is C; — is a bond; D is —CH₂—; L is —C(O)N(R₇)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

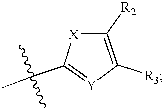

R₂ and R₃ are hydrogen; X is S; Y is N; Z is C; — is a bond; D is —CH(CH₃)—; L is —C(O)N(R₇)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

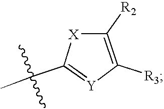

R₂ and R₃ are hydrogen; X is O; Y is N; Z is C; — is a bond; D is —CH₂—; L is —C(O)N(R₇)—; and R₇ and R_{A} are as defined in formula (II).

In another embodiment of the present invention, compounds of formula (II) are disclosed wherein A is aryl wherein the aryl is phenyl substituted with 0, 1, 2, 3, 4, or 5 substituents selected from alkoxy, alkoxycarbonyl, alkyl, cyano, halogen, haloalkoxy, haloalkyl, or nitro; B is

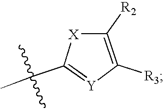

R₂ and R₃ are hydrogen; X is O; Y is N; Z is C; — is a bond; D is —CH(CH₃)—; L is —C(O)N(R₇)—; and R₇ and R_{A} are as defined in formula (II).

In another embodiment of the present invention, compounds of formula (II) are disclosed wherein A is cycloalkyl; B is

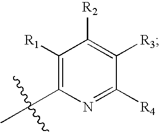

Z is C; — is a bond; L is —C(O)N(R₇)—; and D, R₁, R₂, R₃, R₄, R₇, and R_{A} are as defined in formula (II).

In another embodiment of the present invention, compounds of formula (II) are disclosed wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

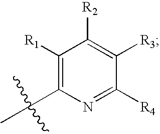

R₁ is selected from hydrogen, alkyl, cyano, or halogen; R₂, R₃, and R₄ are hydrogen; Z is C; — is a bond; D is —CH₂—; L is —C(O)N(R₇)—; and R₇ and R_{A} are as defined in formula (II).

In another embodiment of the present invention, compounds of formula (II) are disclosed wherein A is cycloalkyl wherein the cycloalkyl is selected from cyclohexyl or adamantyl wherein the cycloalkyl is independently substituted with 0, 1, 2, or 3 substituents selected from alkoxy, alkoxycarbonyl, alkyl, halogen, haloalkoxy, or haloalkyl; B is

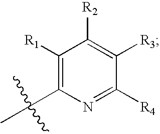

R₁ is selected from hydrogen, alkyl, cyano, or halogen; R₂, R₃, and R₄ are hydrogen; Z is C; — is a bond; D is —CH(CH₃)—; L is —C(O)N(R₇)—; and R₇ and R_{A} are as defined in formula (II).

In another embodiment, the present invention relates to method of treating sexual dysfunction in a mammal comprising administering to the mammal a therapeutically effective amount of a compound of formula (I) or a pharmaceutically acceptable salt, ester, amide, or prodrug thereof in combination with a pharmaceutically acceptable carrier.

In another embodiment, the present invention relates to method of treating sexual dysfunction in a mammal comprising administering to the mammal a therapeutically effective amount of a compound of formula (I) or a pharmaceutically acceptable salt, ester, amide, or prodrug thereof in combination with a phosphodiesterase 5 inhibitor.

In another embodiment, the present invention relates to method of treating sexual cdysfunction in a mammal comprising administering to the mammal a therapeutically effective amount of a compound of formula (I) or a pharmaceutically acceptable salt, ester, amide, or prodrug thereof in combination with an adrenergic receptor antagonist.

In another embodiment, the present invention relates to method of treating sexual dysfunction in a mammal comprising administering to the mammal a therapeutically effective amount of a compound of formula (I) or a pharmaceutically acceptable salt, ester, amide, or prodrug thereof in combination with a dopamine agonist.

In another embodiment, the present invention relates to method of treating male erectile dysfunction in a male human comprising administering to the male human in need of such treatment a therapeutically effective amount of a compound of formula (I) or a pharmaceutically acceptable salt, ester, amide, or prodrug thereof in combination with a pharmaceutically acceptable carrier.

In another embodiment, the present invention relates to method of treating male erectile dysfunction in a mammal comprising administering to the mammal a therapeutically effective amount of a compound of formula (I) or a pharmaceutically acceptable salt, ester, amide, or prodrug thereof in combination with a phosphodiesterase 5 inhibitor.

In another embodiment, the present invention relates to method of treating male erectile dysfunction in a mammal comprising administering to the mammal a therapeutically effective amount of a compound of formula (I) or a pharmaceutically acceptable salt, ester, amide, or prodrug thereof in combination with an adrenergic receptor antagonist.

In another embodiment, the present invention relates to method of treating male erectile dysfunction in a mammal comprising administering to the mammal a therapeutically effective amount of a compound of formula (I) or a pharmaceutically acceptable salt, ester, amide, or prodrug thereof in combination with a dopamine agonist.

In another embodiment, the present invention relates to method of treating female sexual dysfunction in a mammal comprising administering to the mammal in need of such treatment a therapeutically effective amount of a compound of formula (I) or a pharmaceutically acceptable salt, ester, amide, or prodrug thereof in combination with a pharmaceutically acceptable carrier.

In another embodiment, the present invention relates to method of treating female sexual dysfunction in a mammal comprising administering to the mammal a therapeutically effective amount of a compound of formula (I) or a pharmaceutically acceptable salt, ester, amide, or prodrug thereof in combination with a phosphodiesterase 5 inhibitor.

In another embodiment, the present invention relates to method of treating female sexual dysfunction in a mammal comprising administering to the mammal a therapeutically effective amount of a compound of formula (I) or a pharmaceutically acceptable salt, ester, amide, or prodrug thereof in combination with an adrenergic receptor antagonist.

In another embodiment, the present invention relates to method of treating female sexual dysfunction in a mammal comprising administering to the mammal a therapeutically effective amount of a compound of formula (I) or a pharmaceutically acceptable salt, ester, amide, or prodrug thereof in combination with a dopamine agonist.

In another embodiment, the present invention relates to method of treating a disorder selected from cardiovascular disorders, inflammatory disorders, attention deficit hyperactivity disorder, Alzheimer's disease, drug abuse, Parkinson's disease, schizophrenia, anxiety, mood disorders or depression in a mammal comprising administering to the mammal in need of such treatment a therapeutically effective amount of a compound of formula (I) or a pharmaceutically acceptable salt, ester, amide, or prodrug thereof.

### DEFINITIONS OF THE PRESENT INVENTION

As used throughout this specification and the appended claims, the following terms have the following meanings:

The term “alkenyl” as used herein, means a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to e the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxycarbonyl” as used herein, means an alkoxy group, as defined herein, appended to the parent molecular moiety through a carbonyl group, as defined herein. Representative examples of alkoxycarbonyl include, but are not limited to, methoxycarbonyl, ethoxycarbonyl, and tert-butoxycarbonyl.

The term “alkoxysulfonyl” as used herein, means an alkoxy group, as defined herein, appended appended to the parent molecular moiety through a sulfonyl group, as defined herein. Representative examples of alkoxysulfonyl include, but are not limited to, methoxysulfonyl, ethoxysulfonyl and propoxysulfonyl.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylcarbonyl” as used herein, means an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl, and 1-oxopentyl.

The term “alkylcarbonyloxy” as used herein, means an alkylcarbonyl group, as defined herein, appended to the parent molecular moiety through an oxygen atom. Representative examples of alkylcarbonyloxy include, but are not limited to, acetyloxy, ethylcarbonyloxy, and tert-butylcarbonyloxy.

The term “alkylene” means a divalent group derived from a straight or branched chain hydrocarbon of from 1 to 10 carbon atoms. Examples are —CH₂—, —CH₂CH₂—, —CH(CH₃)—, —CH(CH₂CH₃)—, —CH₂CH₂CH₂—, and —CH₂CH₂CH₂CH₂—.

The term “alkylsulfinyl” as used herein, means an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfonyl” as used herein, means an alkyl group, as defined herein, appended to the parent molecular moiety through a sulfonyl group, as defined herein. Representative examples of alkylsulfonyl include, but are not limited to, methylsulfonyl and ethylsulfonyl.

The term “alkylthio” as used herein, means an alkyl group, as defined herein, appended to the parent molecular moiety through a sulfur atom. Representative examples of alkylthio include, but are not limited, methylsulfanyl, ethylsulfanyl, tert-butylsulfanyl, and hexylsulfanyl.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl” as used herein, means a phenyl group, or a bicyclic fused ring system, or a tricyclic fused ring system wherein one or more of the fused rings is a phenyl group. Bicyclic fused ring systems are exemplified by a phenyl group fused to another phenyl group or fused to a cycloalkyl group wherein the cycloalkyl group is selected from cyclopentane, cycloahexane, cycloheptane, or cyclooctane. Tricyclic fused ring systems are exemplified by a bicyclic fused ring system fused to a phenyl group. Representative examples of aryl include, but are not limited to, anthracenyl, azulenyl, fluorenyl, 5,6,7,8-tetrahydronaphthalene, indanyl, indenyl, naphthyl, and phenyl.

The aryl groups of the present invention are substituted with 0, 1, 2, 3, 4, or 5 substituents independently selected from alkoxy, alkenyl, alkyl, alkylsulfinyl, alkylsulfonyl, alkylthio, alkynyl, alkoxycarbonyl, alkylcarbonyl, alkylcarbonyloxy, carboxy, cyano, formyl, halogen, haloalkoxy, haloalkyl, hydroxy, hydroxyalkyl, mercapto, nitro, —NZ₁Z₂, (NZ₃Z₄)carbonyl, and (NZ₃Z₄)sulfonyl;

The term “arylalkyl” as used herein, means an aryl group, as defined herein, appended to the parent molecular moiety through an alkyl group, as defined herein. Representative examples of arylalkyl include, but are not limited to, phenylmethyl, 2-phenylethyl, 3-phenylpropyl, and 3-(2-methylphenyl)propyl.

The term “arylsulfonyl” as used herein, means an aryl group, as defined herein, appended to the parent molecular moiety through a sulfonyl group, as defined herein. Representative examples of arylsulfonyl include, but are not limited to, phenylsulfonyl, 2-methylphenylsulfonyl, 2-nitrophenylsulfonyl, and 3-nitrophenylsulfonyl.

The term “arylalkylsulfonyl” as used herein, means an arylalkyl group, as defined herein, appended to the parent molecular moiety through a sulfonyl group, as defined herein. Representative examples of arylalkylsulfonyl include, but are not limited to, (phenylmethyl)sulfonyl, (2-phenylethyl)sulfonyl, and (3-phenylpropyl)sulfonyl.

The term “carbonyl” as used herein, means a —C(O)— group.

The term “carboxy” as used herein, means a —CO₂H group.

The term “cyano” as used herein, means a —CN group.

The term “cycloalkyl” as used herein, means a monocyclic, bicyclic, or tricyclic ring system. Monocyclic ring systems are exemplified by a saturated cyclic hydrocarbon group containing from 3 to 8 carbon atoms. Examples of monocyclic ring systems include cyclopropyl, cyclobutyl, cyclopentyl, cyclohexyl, cycloheptyl, and cyclooctyl. Bicyclic ring systems are exemplified by a bridged monocyclic ring system in which two non-adjacent carbon atoms of the monocyclic ring are linked by an alkylene bridge of between one and three additional carbon atoms (—CH₂—, —CH₂CH₂—, and —CH₂CH₂CH₂—). Representative examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane. Tricyclic ring systems are exemplified by a bicyclic ring system in which two non-adjacent carbon atoms of the bicyclic ring are linked by a bond or an alkylene bridge of between one and three carbon atoms (—CH₂—, —CH₂CH₂—, and —CH₂CH₂CH₂—). Representative examples of tricyclic-ring systems include, but are not limited to, tricyclo[3.3.1.0^{3.7}]nonane and tricyclo[3.3.1.1^{3.7}]decane (adamantane).

The cycloalkyl groups of the present invention are substituted with 0, 1, 2, 3, or 4 substituents independently selected from alkoxy, alkenyl, alkyl, alkylsulfinyl, alkylsulfonyl, alkylthio, alkynyl, alkoxycarbonyl, alkylcarbonyl, alkylcarbonyloxy, carboxy, cyano, formyl, halogen, haloalkoxy, haloalkyl, hydroxy, hydroxyalkyl, mercapto, nitro, —NZ₁Z₂, (NZ₃Z₄)carbonyl or (NZ₃Z₄)sulfonyl. Representative examples of cycloalkyl substituted with 0, 1, 2, 3, or 4 substituents include, but are not limited to, 2-methylcyclohexyl, 2-cyanocyclohexyl, and 2-methoxycyclohexyl.

The term “fluoroalkylene” as used herein, means at least one fluoride atom (—F) is appended to the parent molecular moiety through an alkylene group, as defined herein. Representative examples of fluoroalkylene are —CH(F)—, —CH(F)CH₂—, —C(F)₂CH₂—, —CH(F)CH(F)—, —CH(CF₃)—, —CH(CH₂CF₃)—, and —CH₂CH₂CH₂CH(F)—.

The term “formyl” as used herein, means a —C(O)H group.

The term “halo” or “halogen” as used herein, refers to —Cl, —Br, —I or —F.

The term “haloalkoxy” as used herein, means at least one halogen, as defined herein, appended to the parent molecular moiety through an alkoxy group, as defined herein. Representative examples of haloalkoxy include, but are not limited to, 2-fluoro-1-chloroethoxy, chloromethoxy, 2-fluoroethoxy, trifluoromethoxy, and pentafluoroethoxy.

The term “haloalkyl” as used herein, means at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “hydroxy” as used herein, means an —OH group.

The term “hydroxyalkyl” as used herein, means at least one hydroxy group, as defined herein, appended to the parent molecular moiety through an alkyl group, as defined herein. Representative examples of hydroxyalkyl include, but are not limited to, hydroxymethyl, 2-hydroxyethyl, 3-hydroxypropyl 2-ethyl-4-hydroxyheptyl and 2,4-dihydroxybutyl.

The term “hydroxyalkylene” as used herein, means at least one hydroxy group, as defined herein, is appended to the parent molecular moiety through an alkylene group, as defined herein. Representative examples of hydroxyalkylene are —CH₂CH(OH)CH₂—, —CH(CH₂OH)—, —CH(CH₂CH₂OH)—, and —CH₂CH₂CH(OH)CH₂—.

The term “mercapto” as used herein, means a —SH group.

The term “nitro” as used herein, means a —NO₂ group.

The term “nitrogen protecting group” as used herein, means those groups intended to protect an amino group against undesirable reactions during synthetic procedures. Nitrogen protecting groups comprise carbamates, amides, N-benzyl derivatives, and imine derivatives. Preferred nitrogen protecting groups are acetyl, benzoyl, benzyl, benzyloxycarbonyl (Cbz), formyl, phenylsulfonyl, pivaloyl, tert-butoxycarbonyl (Boc), tert-butylacetyl, trifluoroacetyl, and triphenylmethyl (trityl).

The term “—NZ₁Z₂” as used herein, means two groups, Z₁ and Z₂, which are appended to the parent molecular moiety through a nitrogen atom. Z₁ and Z₂ are each independently selected from hydrogen, alkyl, alkylcarbonyl, alkylsulfonyl, aryl, arylalkyl, arylalkylsulfonyl, arylsulfonyl, and formyl. Representative examples of —NZ₁Z₂ include, but are not limited to, amino, methylamino, dimethylamino, acetylamino, (acetyl)(methyl)amino, and (methylsulfonyl)amino.

The term “—NZ₃Z₄” as used herein, means two groups, Z₃ and Z₄, which are appended to the parent molecular moiety through a nitrogen atom. Z₃ and Z₄ are each independently selected from hydrogen, alkyl, aryl, or arylalkyl. Representative examples of —NZ₃Z₄ include, but are not limited to, amino, methylamino, dimethylamino, ethylmethylamino, phenylamino, (phenylmethyl)amino, (2-phenylethyl)amino, (phenyl)(methyl)amino, and diethylamino.

The term “(NZ₃Z₄)carbonyl” as used herein, means a —NZ₃Z₄ group, as defined herein, appended to the parent molecular moiety through a carbonyl group, as defined herein. Representative examples of (NZ₃Z₄)carbonyl include, but are not limited to, aminocarbonyl, (methylamino)carbonyl, (dimethylamino)carbonyl, and (phenylmethylamino)carbonyl, ((phenyl)(methyl)amino)carbonyl, (phenylamino)carbonyl, (ethylmethylamino)carbonyl, and (diethylamino)carbonyl.

The term “(NZ₃Z₄)sulfonyl” as used herein, means a —NZ₃Z₄ group, as defined herein, appended to the parent molecular moiety through a sulfonyl group, as defined herein. Representative examples of (NZ₃Z₄)sulfonyl include, but are not limited to, aminosulfonyl, (methylamino)sulfonyl, (dimethylamino)sulfonyl, (phenylmethylamino)sulfonyl, ((phenylmethyl)(methyl)amino)sulfonyl, (phenylmethylamino)sulfonyl, (phenylamino)sulfonyl, and (ethylmethylamino)sulfonyl.

The term “sulfinyl” as used herein, means a —S(O)— group.

The term “sulfonyl” as used herein, means a —S(O)₂— group.

The term “sexual dysfunction” as used herein, means sexual dysfunction in mammals including human male and human female sexual dysfunction.

The term “male sexual dysfunction” as used herein includes, but is not limited to, male erectile dysfunction or premature ejacualtion.

The term “female sexual dysfunction” as used herein includes, but is not limited to, female anorgasmia, clitoral erectile insufficiency, vaginal engorgement, dyspareunia, or vaginismus.

Compounds of the present invention may exist as stereoisomers wherein, asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30. The present invention contemplates various stereoisomers and mixtures thereof and are specifically included within the scope of this invention. Stereoisomers include enantiomers and diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

Compounds of the present invention were named by ACD/ChemSketch version 5.0 (developed by Advanced Chemistry Development, Inc., Toronto, ON, Canada) or were given names which appeared to be consistent with ACD nomenclature.

Preferred compounds of the present invention include:

2-[4-(2-methoxyphenyl)-1-piperazinyl]-N-(3-methylphenyl)acetamide;

2-[4-(2-cyanophenyl)-1-piperazinyl]-N-(3-methylphenyl)acetamide;

N-(3-methylphenyl)-2-[4-(2-pyrimidinyl)-1-piperazinyl]acetamide;

N-(3-methylphenyl)-2-[4-(2-pyridinyl)-1-piperazinyl]acetamide;

2-[4-(3-cyano-2-pyridinyl)-1-piperazinyl]-N-(3-methylphenyl)acetamide;

N-(3 methylphenyl)-2-[4-(2-methylphenyl) 1-piperazinyl]acetamide;

N-(3-methylphenyl)-2-[4-(2-nitrophenyl)-1-piperazinyl]acetamide;

2-[4-(3-cyano-2-pyridinyl)-1-piperazinyl]-N-(3-nitrophenyl)acetamide;

2-[4-(3-cyano-2-pyridinyl)-1-piperazinyl]-N-[3-(trifluoromethyl)phenyl]acetamide;

N-(3-methylphenyl)-2-(4-phenyl-1-piperazinyl)acetamide;

N-(3-cyanophenyl)-2-[4-(3-cyano-2-pyridinyl)-1-piperazinyl]acetamide;

N-(4-bromo-3-methylphenyl)-2-[4-(2-cyanophenyl)-1-piperazinyl]acetamide;

2-[4-(2-cyanophenyl)-1-piperazinyl]-N-phenylacetamide;

2-[4-(3-cyano-2-pyridinyl)-1-piperazinyl]-N-phenylacetamide;

2-[4-(3-cyano-2-pyridinyl)-1-piperazinyl]-N-(4-fluorophenyl)acetamide;

2-[4-(3-cyano-2-pyridinyl)-1-piperazinyl]-N-(3,5-dimethylphenyl)acetamide;

2-[4-(3-cyano-2-pyridinyl)-1-piperazinyl]-N-(2,3-dimethylphenyl)acetamide;

2-[4-(3-cyano-2-pyridinyl)-1-piperazinyl]-N-(2-methylphenyl)acetamide;

2-[4-(3-cyano-2-pyridinyl)-1-piperazinyl]-N-(2,5-dimethylphenyl)acetamide;

N-(3-chlorophenyl)-2-[4-(3-cyano-2-pyridinyl)-1-piperazinyl]acetamide;

N-(3-chloro-4-fluorophenyl)-2-[4-(3-cyano-2-pyridinyl)-1-piperazinyl]acetamide;

2-[4-(3-cyano-2-pyridinyl)-1-piperazinyl]-N-(3,4,5-trimethoxyphenyl)acetamide;

2-[4-(3-cyano-2-pyridinyl)-1-piperazinyl]-N-[4-fluoro-3-(trifluoromethyl)phenyl] acetamide;

2-[4-(3-cyano-2-pyridinyl)-1-piperazinyl]-N-[3-fluoro-5-(trifluoromethyl)phenyl] acetamide;

2-[4-(3-cyano-2-pyridinyl)-1-piperazinyl]-N-[2-fluoro-5-(trifluoromethyl)phenyl] acetamide;

2-[4-(3-cyano-2-pyridinyl)-1-piperazinyl]-N-[2-fluoro-3-(trifluoromethyl)phenyl] acetamide;

2-[4-(3-cyano-2-pyridinyl)-1-piperazinyl]-N-(4-fluoro-3-methylphenyl)acetamide;

2-[4-(3-cyano-2-pyridinyl)-1-piperazinyl]-N-(2-fluorophenyl)acetamide;

2-[4-(3-cyano-2-pyridinyl)-1-piperazinyl]-N-(2-methoxyphenyl)acetamide;

2-[4-(3-cyano-2-pyridinyl)-1-piperazinyl]-N-(2-nitrophenyl)acetamide;

2-[4-(3-cyano-2-pyridinyl)-1-piperazinyl]-N-[2-(trifluoromethyl)phenyl]acetamide;

N-phenyl-2-[4-(2-pyridinyl)-1-piperazinyl]acetamide;

N-(3-methylphenyl)-2-[4-(1,3-thiazol-2-yl)-1-piperazinyl]acetamide;

2-[4-(3-cyano-2-pyridinyl)-1-piperazinyl]-N-(4-methylphenyl)acetamide;

2-[4-(2-methoxyphenyl)-1-piperidinyl]-N-(3-methylphenyl)acetamide;

2-[4-(2-fluorophenyl)-1-piperidinyl]-N-(3-methylphenyl)acetamide;

N-(3-methylphenyl)-2-[4-(2-methylphenyl)-1-piperidinyl]acetamide;

2-[4-(3-fluorophenyl)-1-piperidinyl]-N-(3-methylphenyl)acetamide;

N-(3-methylphenyl)-2-[4-(6-oxo-1(6H)-pyridazinyl)-1-piperidinyl]acetamide;

N-(2,6-dimethylphenyl)-2-[4-(2-thienyl)-1-piperidinyl]acetamide;

N-(2,5-dimethylphenyl)-2-[4-(2-thienyl)-11-piperidinyl] acetamide;

N-(2-methylphenyl)-2-[4-(2-thienyl)-1-piperidinyl]acetamide;

N-(3-chloro-4-fluorophenyl)-2-[4-(2-thienyl)-1-piperidinyl] acetamide;

N-(4-bromophenyl)-2-[4-(2-pyridinyl)-1-piperidinyl] acetamide;

N-(2,6-dimethylphenyl)-2-[4-(2-pyridinyl)-1-piperidinyl]acetamide;

N-(2-nitrophenyl)-2-[4-(2-pyridinyl)-1-piperidinyl]acetamide;

N-(3-nitrophenyl)-2-[4-(2-pyridinyl)-1-piperidinyl]acetamide;

N-(2,4-difluorophenyl)-2-[4-(2-pyridinyl)-1-piperidinyl]acetamide;

N-(2,5-dimethylphenyl)-2-[4-(2-pyridinyl)-1-piperidinyl]acetamide;

N-(2-methylphenyl)-2-[4-(2-pyridinyl)-1-piperidinyl] acetamide;

N-(4-methylphenyl)-2-[4-(2-pyridinyl)-1-piperidinyl]acetamide; 2-[4-(2-pyridinyl)-1-piperidinyl]-N-[3-(trifluoromethyl)phenyl] acetamide;

ethyl 4-({[4-(2-pyridinyl)-1-piperidinyl]acetyl}amino)benzoate;

N-(3-chloro-4-methylphenyl)-2-[4-(2-pyridinyl)-1-piperidinyl]acetamide;

N-(2-cyanophenyl)-2-[4-(2-pyridinyl)-1-piperidinyl] acetamide;

N-(3-chlorophenyl)-2-[4-(2-pyridinyl)-1-piperidinyl]acetamide;

2-[4-(3-cyano-2-pyridinyl)-1-piperidinyl]-N-(3-methylphenyl)acetamide;

N-(3-methylphenyl)-2-(4-phenyl-3,6-dihydro-1 (2H)-pyridinyl)acetamide;

2-(3′,6′-dihydro-2,4′-bipyridin-1′(2′H)-yl)-N-(3-methylphenyl)acetamide;

2-(3′,6′-dihydro-2,4′-bipyridin-1′(2′H)-yl)-N-(2,6-dimethylphenyl)acetamide;

2-(3′,6′-dihydro-2,4′-bipyridin-1′(2′H)-yl)-N-(2-nitrophenyl)acetamide;

2-(3′,6′-dihydro-2,4′-bipyridin-1′(2′H)-yl)-N-(4-fluorophenyl)acetamide;

N-(2,4-difluorophenyl)-2-(3′,6′-dihydro-2,4′-bipyridin-1′(2′H)yl)acetamide;

2-(3′,6′-dihydro-2,4′-bipyridin-1-(2′)-yl)-N-(2,5-dimethylphenyl)acetamide;

2-(3′,6′-dihydro-2,4′-bipyridin-1′(2′H)-yl)-N-(4-fluorophenyl)acetamide;

N(2,4-diflourophenyl)-cyclohexyl-2-(3′,6′-dihydro-2,4′-bipyridin-1′(2′ H)-yl) acetamide;

2-(3′,6′-dihydro-2,4′-bipyridin-1′(2′H)-yl)-N-(4-methylphenyl)acetamide;

2-(3′,6′-dihydro-2,4′-bipyridin-1′(2′H)-yl)-N-[3-(trifluoromethyl)phenyl]acetamide;

ethyl 4-[(3′,6′-dihydro-2,4′-bipyridin-1′-((1H)-ylacety)amino]benzoate;

N-[2-chloro-5-(trifluoromethyl)phenyl]-2-(3′,6′-dihydro-2,4′-bipyridin-1,2′H)-yl)acetamide;

N-(3-chloro-4-methylphenyl)-2-(3′,6′-dihydro-2,4′-bipyridin-1′(2′H)-yl)acetamide;

N-(2-cyanophenyl)-2-(3′,6′-dihydro-2,4′-bipyridin 1′(2′H)-yl) acetamide;

N-(3-chlorophenyl)-2-(3′,6′-dihydro-2,4′-bipyridin-1′(2′H)-yl)acetamide;

N-(3-chloro-4-fluorophenyl)-2-(3′,6′-di hydro-2,4′-bipyridin-1′(2′H)-yl)acetamide;

2-(3′,6-dihydro-2,4′-bipyridin-1′(2′H)-yl)-N-[4-(trifluoromethoxy)phenyl]acetamide;

2-(3′,6′-dihydro-2,4′-bipyridin 1′(2′H)-yl)-N-[2-(trifluoromethyl)phenyl]acetamide;

N-(4-chlorophenyl)-2-(3′,6′-dihydro-2,4′-bipyridin-1′(2′H)-yl)acetamide;

N-(2,3-dichlorophenyl)-2-(3′,6′-dihydro-2,4′-bipyridin-1′(2′H)-yl)acetamide;

N-(3,5-dichlorophenyl)-2-(3′,6′-dihydro-2,4′-bipyridin-1′(2′H)-yl)acetamide;

2-(3′,6′-dihydro-2,4′-bipyridin-1′(2′H)-yl)-N-(4-fluoro-2-methylphenyl)acetamide;

N-(4-fluorophenyl)-2-[4-(2-pyridinyl)-1-piperidinyl]acetamide;

N-(3,5-dichlorophenyl)-2-[4-(2-pyridinyl)-1-piperidinyl]acetamide;

N-(2,3-dichlorophenyl)-2-[4-(2-pyridinyl)-1-piperidinyl]acetamide;

2-[4-(2-pyridinyl)-1-piperidinyl]-N-[2-(trifluoromethyl)phenyl]acetamide;

N-(3-chloro-4-fluorophenyl)-2-[4-(2-pyridinyl)-1-piperidinyl]acetamide;

2-[4-(2-pyridinyl)-1-piperidinyl]-N-[4-(trifluoromethoxy)phenyl]acetamide;

N-Cyclohexyl-2-(3′,4′,5′,6′-tetrahydro-2′H-[2,4′]bipyridinyl-1′-yl) acetamide;

N-{[4-(2-cyanophenyl)-1-piperazinyl]methyl}-3-methylbenzamide;

3-methyl-N-{[4-(2-pyrimidyl)-1-piperazinyl]methyl}benzamide;

3-methyl-N-{[4-(2-pyridinyl)-1-piperazinyl]methyl}benzamide;

3-methyl-N-[(4-phenyl)1-piperazinyl)methyl]ₘ benzamide;

N-{[4-(2-methoxyphenyl)-1-piperazinyl]methyl}-3-methylbenzamide;

N-{[4-(2-cyanophenyl)-1-piperazinyl]methyl-2-methylbenzamide;

N-{[4-(2-cyanophenyl)-1-piperazinyl]methyl}-4-methylbenzamide;

N-{[4-(3-cyano-2-pyridinyl)-1-piperazinyl]methyl}-3-methylbenzamide;

N-{[4-(3-cyanophenyl)-1-piperazinyl]methyl}-3-methylbenzamide;

N-{[4-(3-cyanophenyl)-1-piperazinyl]methyl}-2-methylbenzamide;

N-{[4-(3-cyano-2-pyridinyl)-1-piperazinyl]methyl} benzamide;

N-[4-(3-cyano-2-pyridinyl)-1-piperazinylmethyl}-4-methylbenzamide;

N-{[4-(3-cyano-2-pyridinyl)-1-piperazinyl]methyl}-2-methylbenzamide;

N-{[4-(2-pyridinyl)-1-piperazinyl]methyl}benzamide;

N-{[4-(2-chlorophenyl)-1-piperazinyl]methyl}benzamide;

3-chloro-N-{[4-(2-cyanophenyl)-1-piperazinyl]methyl}benzamide;

4-chloro-N-{[4-(2-methoxy phenyl)-1-piperazinyl]methyl}benzamide;

2-chloro-N-{[4-(3-cyano-2-pyridinyl)-1-piperazinyl]methyl}benzamide;

N-{[4-(3-cyano-2-pyridinyl) 1-piperazinyl]methyl}-2-(trifluoromethyl)benzamide;

N-{[4-(2-cyanophenyl) 1-piperazinyl]methyl}benzamide;

N-{[4-(2-methoxyphenyl)-1-piperidinyl]methyl}-3-methylbenzamide;

3-methyl-N-{[4-(2-pyridinyl)-1-piperidinyl]methyl}benzamide;

3-methyl-N-[(4-phenyl-3,6-dihydro-1 (2H)-pyridinyl)methyl]benzamide;

N-(3′,6′-dihydro-2,4′-bipyridin-1′(2′H)-ylmethyl)-3-methylbenzamide;

N-(3′, 6′-dihydro-2,4′-bipyridin-1′(2′H)-ylmethyl)-3-methoxybenzamide;

N-(3′,6′-dihydro-2,4′-bipyridin-1′(2′H)-ylmethyl)-3-fluorobenzamide;

N-(3′,6′-dihydro-2,4′-bipyridin-1′(2′H)-ylmethyl)-3,5-difluorobenzamide;

2-[4-(3-cyano-2-pyridinyl)-1-piperazinyl]-N-3-pyridinylacetamide;

2-(1-{2-[(3-methylphenyl)amino]-2-oxoethyl}piperidin-4-yl)pyridinium N-oxide;

N-(3-methylphenyl)-2-[4-(2-pyridinyl)-1-piperidinyl]acetamide;

N-2-adamantyl-2-[4-(3-cyano-2-pyridinyl)-1-piperazinyl]acetamide;

2-[4-(3-cyano-2-pyridinyl)-1-piperazinyl]-N-cyclohexylacetamide;

2-[4-(3-cyano-2-pyridinyl)-1-piperazinyl]-N-5,6,7,8-tetrahydro-1-naphthalenylacetamide;

2-(3′,6′-dihydro-2,4′-bipyridin-1′(2′H)-yl)-N-(4-fluoro-2-methylphenyl)acetamide;

N-{[4-(2-pyridinyl)-1-piperidinyl]methyl}-3-(trifluoromethyl)benzamide;

3,5-dimethoxy-N-{[4-(2-pyridinyl)-1-piperidinyl]methyl}benzamide;

N-{[4-(2-pyridinyl)-1-piperidinyl]methyl} cyclohexanecarboxamide;

3,4-difluoro-N-{[4-(2-pyridinyl)-1-piperidinyl]methyl}benzamide;

3-chloro-N-[4-(2-pyridinyl)-1-piperidinyl]methyl)benzamide;

2,3-dimethyl-N-{[4-(2-pyridinyl)-1-piperazinyl]methyl}benzamide;

N-(3′,6′-dihydro-2,4′-bipyridin-1′(2′H)-ylmethyl)-3-(trifluoromethyl)benzamide;

3-chloro-N-(3′,6′-dihydro-2,4′-bipyridin-1′(2′H)-ylmethyl)benzamide;

N-(3′,6′-dihydro-2,4′-bipyridin-1′(2′H)-ylmethyl)cyclohexanecarboxamide;

N-(3′,6′-dihydro-2,4′-bipyridin-1′(2′H)-ylmethyl)-3,4-difluorobenzamide;

N-(3′,6′-dihydro-2,4′-bipyridin-1′(2′H)-ylmethyl)-3,5-dimethoxybenzamide;

N-(3-methylphenyl)-2-(4-phenyl-1-piperidinyl)acetamide;

2-(3′,6′-dihydro-2,4′-bipyridin-1′(2′H)-yl)-N-(3-nitrophenyl)acetamide;

N-1-adamantyl-2-[4-(3-cyano-2-pyridinyl)-1-piperazinyl] acetamide;

3-methyl-N-{[2-methyl-4-(2-pyridinyl)-1-piperazinyl]methyl}benzamide;

N-(3-methylphenyl)-2-[2-methyl-4-(2-pyridinyl)-1-piperazinyl]acetamide;

3,5-dimethyl-N-{[4-(2-pyridinyl)-1-piperidinyl]methyl}benzamide;

N-(3′,6′-dihydro-2,4′-bipyridin-1′(2′H)-ylmethyl)-3,5-dimethylbenzamide;

3-methyl-N-[(3-methyl-3′,6′-dihydro-2,4′-bipyridin-1′(2′H)-yl)methyl]benzamide;

N-[(3-cyano-3′,6′-dihydro-2,4′-bipyridin-1′(2′H)-yl)methyl]-3-methylbenzamide;

N-(2,6-dimethylphenyl)-2-(3-methyl-3′,6′-dihydro-2,4′-bipyridin-1′(2′H)-yl)acetamide;

N-(4-fluorophenyl)-2-(3-methyl-3′,6′-dihydro-2,4′-bipyridin-1′(2′H)-yl)acetamide;

N-(2,4-difluorophenyl)-2-(3-methyl-3′,6′-dihydro-2,4′-bipyridin-1′(2′H)-yl)acetamide;

2-(3-methyl-3′,6′-dihydro-2,4′-bipyridin-1′(2′H)-yl)-N-(2-methylphenyl)acetamide;

2-(3-methyl-3′,6′-dihydro-2,4′-bipyridin-1′(2′H)-yl)-N-[3-(trifluoromethyl)phenyl] acetamide;

N-(3-chloro-4-fluorophenyl)-2-(3-methyl-3′,6′-dihydro-2,4′-bipyridin-1′(2′H)-yl)acetamide;

2-(3-methyl-3′,6′-dihydro-2,4′-bipyridin-1′(2′H)-yl)-N-[4-(trifluoromethoxy)phenyl]acetamide;

2-(3-methyl-3′,6′-dihydro-2,4′-bipyridin-1′(2′H)-yl)-N-[2-(trifluoromethyl)phenyl]acetamide;

N-(2,3-dichlorophenyl)-2-(3-methyl-3′,6′-dihydro-2,4′-bipyridin-1′(2′H)-yl)acetamide;

2-(3-methyl-3′,6′-dihydro-2,4′-bipyridin-1′(2′H)-yl)-N-[4-(trifluoromethyl)phenyl]acetamide;

2-[4-(3-cyano-2-thienyl)-3,6-dihydro-1(2H)-pyridinyl]-N-(3-methylphenyl)acetamide;

2-(3-cyano-3′,6′-dihydro-2,4′-bipyridin-1′(2′H)-yl)-N-(2,6-dimethylphenyl)acetamide;

2-(3-cyano-3′,6′-dihydro-2,4′-bipyridin-1′(2′H)-yl)-N-(4-fluorophenyl)acetamide;

2-(3-cyano-3′,6′-dihydro-2,4′-bipyridin-1′(2′H)-yl)-N-(2,4-difluorophenyl)acetamide;

2-(3-cyano-3′,6′-dihydro-2,4′-bipyridin-1′(2′H)-yl)-N-(2-methylphenyl)acetamide;

2-(3-cyano-3′,6′-dihydro-2,4′-bipyridin-1′(2′H)-yl)-N-[3-(trifluoromethyl)phenyl]acetamide;

2-(3-cyano-3′,6′-dihydro-2,4′-bipyridin-1′(2′H)-yl)-N-[4-(trifluoromethoxy)phenyl]acetamide;

2-(3-cyano-3′,6′-dihydro-2,4′-bipyridin-1′(2′H)-yl)-N-[2-(trifluoromethyl)phenyl]acetamide;

2-(3-cyano-3′,6′-dihydro-2,4′-bipyridin-1′(2′H)-yl)-N-(2,3-dichlorophenyl)acetamide;

3-methyl-N-{[4-(6-oxo-1(6H)-pyridazinyl)-1-piperidinyl]methyl}benzamide;

N-(3′,6′-dihydro-2,4′-bipyridin-1′(2′H)-ylmethyl)-1-adamantanecarboxamide;

3-methyl-N-{[4-(1,3-thiazol-2-yl)-3,6-dihydro-1 (2H)-pyridinyl]methyl}benzamide;

2-[4-(3-cyano-2-pyridinyl)-1-piperazinyl]-N-1,2,3,4-tetrahydro-1-naphthalenylacetamide;

2-[4-(3-cyano-2-pyridinyl)-1-piperazinyl]-N-[(1S)-1,2,3,4-tetrahydro-1-naphthalenyl]acetamide;

2-[4-(3-cyano-2-pyridinyl)-1-piperazinyl]-N-[(1R)-1,2,3,4-tetrahydro-1-naphthalenyl] acetamide;

N-(2,6-diethylphenyl)-2-[4-(2-pyridinyl)-1-piperidinyl]acetamide;

2-[4-(2-pyridinyl)-1-piperidinyl]-N-(2,4,6-trifluorophenyl)acetamide;

N-(4-chloro-2,6-dimethylphenyl)-2-[4-(2-pyridinyl)-1-piperidinyl]acetamide;

2-[4-(2-pyridinyl)-1-piperidinyl]-N-(2,4,6-trichlorophenyl)acetamide;

N-(2,6-diethylphenyl)-2-(3′,6′-dihydro-2,4′-bipyridin-1′(2′H)-yl)acetamide;

2-(3′,6′-dihydro-2,4′-bipyridin-1′(2′H)-yl)-N-(2,4,6-trifluorophenyl)acetamide;

N-(4-chloro-2,6-dimethylphenyl)-2-(3′,6′-dihydro-2,4′-bipyridin-1′(2′H)-yl)acetamide;

2-(3′,6′-dihydro-2,4′-bipyridin-1′(2′H)-yl)-N-(2,4,6-trichlorophenyl)acetamide;

N-{[4-(2-pyridinyl)-1-piperazinyl]methyl}-3-(trifluoromethyl)benzamide;

3,5-dimethoxy-N-{[4-(2-pyridinyl)-1-piperazinyl]methyl}benzamide;

N-{[4-(2-pyridinyl)-1-piperazinyl]methyl} cyclohexanecarboxamide;

N-(2,6-dimethylphenyl)-2-[4-(2-pyridinyl)-1-piperazinyl]acetamide;

N-(4-fluorophenyl)-2-[4-(2-pyridinyl)-1-piperazinyl]acetamide;

N-(2,4-difluorophenyl)-2-[4-(2-pyridinyl)-1-piperazinyl]acetamide;

N-(2-methylphenyl)-2-[4-(2-pyridinyl)-1-piperazinyl]acetamide;

2-[4-(2-pyridinyl)-1-piperazinyl]-N-[3-(trifluoromethyl)phenyl]acetamide;

N-(3-chlorophenyl)-2-[4-(2-pyridinyl)-1-piperazinyl]acetamide;

N-benzyl-2-[4-(2-pyridinyl)-1-piperazinyl]acetamide;

2-[4-(2-pyridinyl)-1-piperazinyl]-N-[4-(trifluoromethoxy)phenyl]acetamide;

2-[4-(2-pyridinyl)-1-piperazinyl]-N-[2-(trifluoromethyl)phenyl]acetamide;

N-(4-chlorophenyl)-2-[4-(2-pyridinyl)-1-piperazinyl]acetamide;

N-(2,3-dichlorophenyl)-2-[4-(2-pyridinyl)-1-piperazinyl]acetamide;

N-(3,4-dichlorophenyl)-2-[4-(2-pyridinyl)-1-piperazinyl]acetamide;

2-[4-(2-pyridinyl)-1-piperazinyl]-N-[4-(trifluoromethyl)phenyl] acetamide;

3-chloro-N-{[4-(2-pyridinyl)-1-piperazinyl]methyl}benzamide;

4-fluoro-3-methyl-N-{[4-(2-pyridinyl)-1-piperazinyl]methyl}benzaniide;

N-(3′,6′-dihydro-2,4′-bipyridin-1′(2′H)-ylmethyl)-4-fluoro-3-methylbenzamide;

3-methyl-N-{[4-(1,3-oxazol-2-yl)-3,6-dihydro-1 (2H)-pyridinyl]methyl}benzamide;

2-methyl-N-[(3-methyl-3′,6′-dihydro-2,4′-bipyridin-1′(2′H)-yl)methyl]benzamide;

2-[4-(3-cyano-2-pyridinyl)-1-piperidinyl]-N-(2,6-dimethylphenyl)acetamide;

2-(1-{[(3-methylbenzoyl)amino]methyl}-4-piperidinyl)pyridinium N-oxide;

N-(3-methylphenyl)-2-[4-(3-methyl-2-pyridinyl)-1-piperazinyl]acetamide;

2-[4-(3-cyano-2-pyridinyl)-1-piperazinyl]-N-[4-(trifluoromethyl)phenyl]acetamide;

N-(2-ethyl-6-methylphenyl)-2-[4-(2-pyridinyl)-1-piperidinyl]acetamide;

N-(2-isopropyl-6-methylphenyl)-2-[4-(2-pyridinyl)-1-piperidinyl]acetamide;

N-(2-chloro-6-methylphenyl)-2-[4-(2-pyridinyl)-1-piperidinyl]acetamide;

N-(2-methoxy-6-methylphenyl)-2-[4-(2-pyridinyl)-1-piperidinyl]acetamide;

2-(32′,6′-dihydro-2,4′-bipyridin-(2-pyridnyl)-pipperidinylacetamide;

2-(3′,6′-dihydro-2,4′-bipyridin-1′(2′H)-yl)-N-(2-isopropyl-6-methylphenyl)acetamide;

N-(2-chloro-6-methylphenyl)-2-(3′,6′-dihydro-2,4′-bipyridin-1′(2′H)-yl)acetamide;

2-(3′,6′-dihydro-2,4′-bipyridin-11′(2′H)-yl)-N-(2-methoxy-6-methylphenyl)acetamide;

3-chloro-N-[(3-methyl-3′,6′-dihydro-2,4′-bipyridin-1′(2′H)-yl)methyl]benzamide;

3-fluoro-N-[(3-methyl-3′,6′-dihydro-2,4′-bipyridin-1′(2′H)-yl)methyl]benzamide;

3-methyl-N-{[(2S)-2-methyl-4-(2-pyridinyl)-1-piperazinyl]methyl}benzamide;

N-(3-methylphenyl)-2-[(2S)-2-methyl-4-(2-pyridinyl)-1-piperazinyl]acetamide;

3-methyl-N-{[(2R)-2-methyl-4-(2-pyridinyl)-1-piperazinyl]methyl}benzamide;

N-(3-methylphenyl)-2-[(2R)-2-methyl-4-(2-pyridinyl)-1-piperazinyl]acetamide;

3-methoxy-N-[(3-methyl-3′,6′-dihydro-2,4′-bipyridin-1′(2′H)-yl)methyl]benzamide;

4-fluoro-N-[(3-methyl-3′,6′-dihydro-2,4′-bipyridin-1′(2′H)-yl)methyl]benzamide;

2-(3-chloro-3′,6′-dihydro-2,4′-bipyridin-1′(2′H)-yl)-N-(2,6-dimethylphenyl)acetamide;

2-(3-chloro-3′,6′-dihydro-2,4′-bipyridin-1′(2′H)-yl)-N-(2-methylphenyl)acetamide;

N-(3′,6′-dihydro-2,4′-bipyridin-1′(2′H)-ylmethyl)-1-naphthamide;

N-{[4-(3-cyano-2-pyridinyl)-1-piperazinyl]methyl}-3-fluorobenzamide;

3-methyl-N-{[4-(1,3-thiazol-2-yl)-1-piperidinyl]methyl}benzamide;

2-(1-{2-[(4-fluoro-2-methylphenyl)amino]-2-oxoethyl}-4-piperidinyl)pyridinium N-oxide;

2-(1-{2-[(4-fluoro-3-methylphenyl)amino]-2-oxoethyl}-4-piperidinyl)pyridinium N-oxide;

2-(1-{2-[(3-fluorophenyl)amino]-2-oxoethyl}-4-piperidinyl)pyridinium N-oxide;

2-(1-{2-[(2-fluoro-5-methylphenyl)amino]-2-oxoethyl}-4-piperidinyl)pyridinium N-oxide;

2-(1-{1-methyl-2-[(3-methylphenyl)amino]-2-oxoethyl}-4-piperidinyl)pyridinium N-oxide;

2-(2-[(4-fluorophenyl)amino]-2-oxoethyl-4-piperidinyl)pyridinium-oxide; 2-(1-{2-[(2-fluorophenyl)amino]-2-oxoethyl}-4-piperidinyl)pyridinium-N-oxide;

and N-(3-methylphenyl)-2-{4-[3-(trifluoromethyl)-2-pyridinyl]-1-piperazinyl}acetamide or pharmaceutically acceptable salts, esters, amides, or prodrugs thereof.

### Abbreviations

Abbreviations which have been used in the descriptions of the Schemes and the Examples that follow are: Ac for acetyl; BINAP for 2,2′-bis(diphenylphosphino)-1,1′-binaphthyl; Boc for tert-butoxycarbonyl; nBuLi for n-butyllithium; dba for dibenzylideneacetone; DME for dimethoxyethane; DMF for N,N-dimethylformamide; DMSO for dimethylsulfoxide; EtOH for ethanol; HPLC for high pressure liquid chromatography; MeOH for methanol; TEA for triethylamine; TFA for trifluoroacetic acid; THF for tetrahydrofuran; THP for tetrahydropyran; TLC for thin layer chromatography.

Preparation of Compounds of the Present Invention

The compounds and processes of the present invention will be better understood in connection with the following synthetic Schemes and Examples which illustrate a means by which the compounds of the present invention can be prepared.

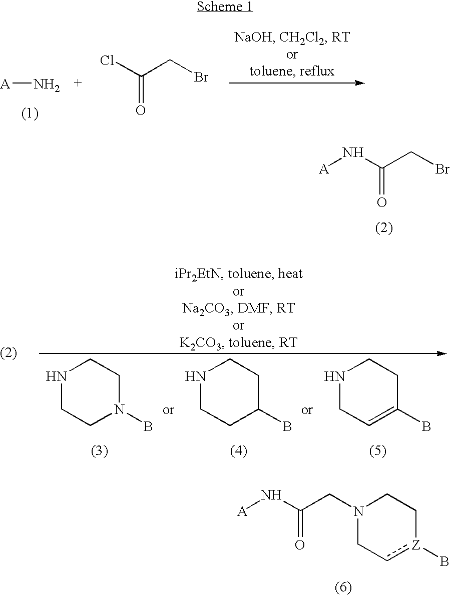

Compound of general formula (6), wherein A, Z, B, and — are as defined in formula (I), can be prepared as described in Scheme 1. Amines of general formula (1) can be treated with bromoacetyl chloride, sodium hydroxide in a solvent such as, but not limited to, toluene or methylene chloride to provide compounds of general formula (2). Compounds of general formula (2) can be treated with an amine of general formula (3) or (4) or (5) in the presence of a base such as, but not limited to, diisopropylethylamine, sodium carbonate, or potassium carbonate in a solvent such as, but not limited to, toluene or N,N-dimethylformamide to provide compounds of general formula (6).

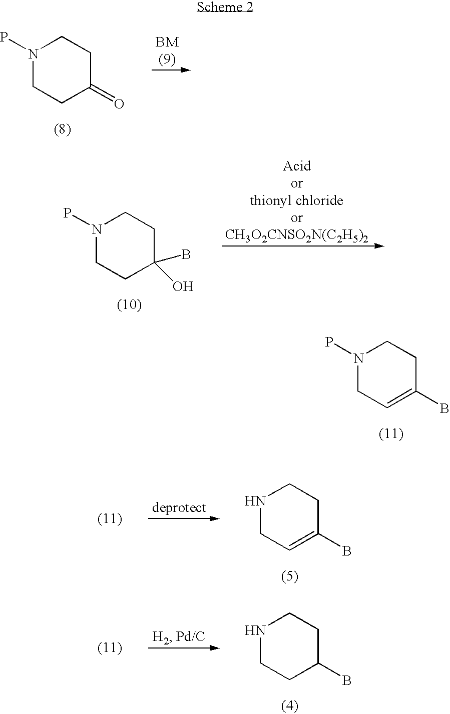

Compounds of general formula (5) and (4), wherein B is as defined in formula (I) can be prepared as described in Scheme 2. Piperidinones of general formula (8), purchased commercially or prepared using standard methods know to those of skill in the art wherein P is a nitrogen protecting group such as, but not limited to, (CH₃)₃CO₂C— or C₆H₅CH₂O₂C—, can be treated with compounds of general formula (9), wherein M is Li, MgBr, MgCl, Cu, or Zn to provide compounds of general formula (10). Compounds of general formula (10) can be treated with Burgess Reagent, thionyl chloride or an acid such as, but not limited to, sulfuric acid or trifluoracetic acid to provide dihyropyridines of general formula (11). Dihyropyridines of general formula (11) can be deprotected using standard methods known to those of ordinary skill in the art to provide compounds of general formula (4) and (5).

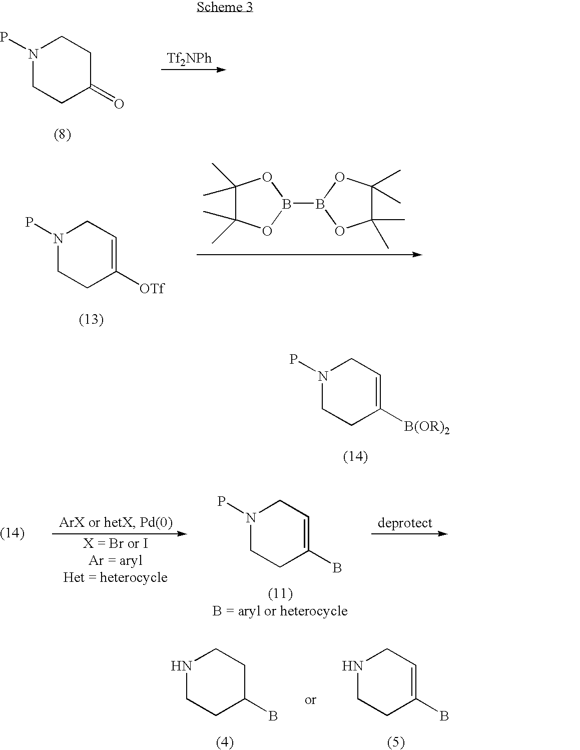

Compounds of general formula (4) and (5), wherein B is as defined in formula (I), can be prepared as described in Scheme 3. Piperidinones of general formula (8), purchased commercially or prepared using standard methods know to those of skill in the art wherein P is a nitrogen protecting group such as, but not limited to, (CH₃)₃CO₂C— or C₆H₅CH₂O₂C—, can be treated with Tf₂NPh to provide triflates of general formula (13). Triflates of general formula (13) can be treated with diborane pinacol ester to provide boranes of general formula (14). Boranes of general formula (14) can be treated with ArX or HetX in the presence of a Pd(0) catalyst to provide compounds of general formula (11). Compounds of general formula (11) can be deprotected using standard methods known to those of ordinary skill in the art to provide compounds of general formula (4) and (5).

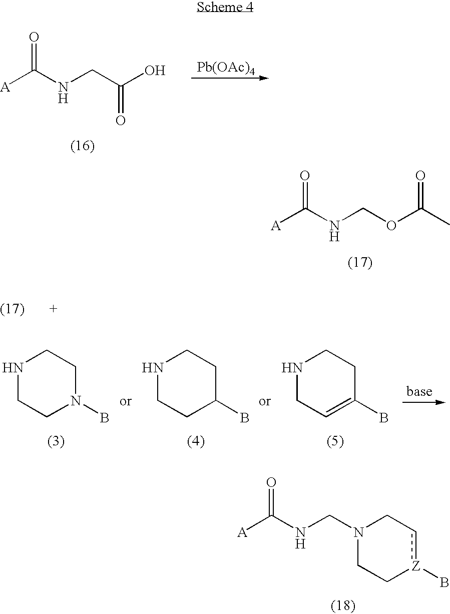

Compounds of general formula (18), wherein A, Z, B, and — are as defined in formula (I), can be prepared as described in Scheme 4. Acids of general formula (16), purchased commercially or prepared using standard methods known to those of ordinary skill in the art, can be treated with lead tetraacetate and copper(II) acetate in a solvent such as, but not limited to, toluene with heat to provide acetates of general formula (17). Acetates of general formula (17) can be treated with an amine of general formula (3) or (4) or (5) and a base such as, but not limited to, triethylamine in a solvent such as, but not limited to, acetonitrile to provide compounds of general formula (18).

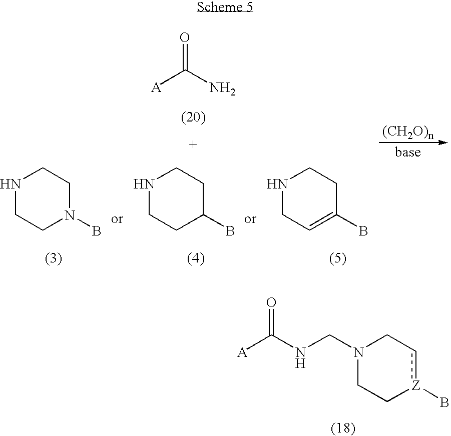

Compounds of general formula (18), wherein A, Z, B, and — are as defined in formula (I), can be prepared as described in Scheme 5. Amides of general formula (20), purchased commercially or prepared using methods known to those of ordinary skill in the art, can be treated with paraformaldehyde and a base such as, but not limited to, potassium carbonate in a solvent such as, but not limited to, ethanol with heat to provide compounds of general formula (18).

The following Examples are intended as an illustration of and not a limitation upon the scope of the invention as defined in the appended claims.

### EXAMPLE 1

### 2-[4-(2-methoxyphenyl)-1-piperazinyl]-N-(3-methylphenyl Acetamide

### EXAMPLE 1A

### 2-bromo-N-(3-methylphenyl)acetamide

3-Methylaniline (Acros, 15.50 mL, 141.8 mmol) in 2N aqueous sodium hydroxide (200 mL) at room temperature was treated with bromoacetyl chloride (Sigma, 12.50 mL, 152.0 mmol) as a solution in dichloromethane (200 mL). After 30 minutes, the layers were separated and the aqueous phase extracted with additional portions of dichloromethane. The organic phases were combined, washed with an aqueous solution of 1N HCl, dried (Na₂SO₄), filtered, and the filtrate concentrated under reduced pressure to provide 16.69 g (52% yield) of the title compound as a white solid. ¹H NMR (300 MHz, DMSO-d₆) δ 2.28 (s, 3H), 4.01 (s, 2H), 6.91 (d, 1H, J=7.5 Hz), 7.20 (dd, 1H, J=7.5, 7.5 Hz), 7.36 (d, 1H, J=8.8 Hz), 7.42 (s, 1H), 10.28 (br s, 1H); MS (DCI/NH₃) m/e 228/230 (M+H)⁺; 245/247 (M+NH₄)⁺.

### EXAMPLE 1B

### 2-[4-(2-methoxyphenyl)-1-piperazinyl]-N-(3-methylphenyl)acetamide

1-(2-methoxyphenyl)piperazine, (Aldrich, 1.50 g, 7.80 mmol) and N,N-diisopropylethylamine (2.0 mL) in toluene (30 mL) were treated with the product from Example 1A (1.12 g, 4.90 mmol) and heated at 60° C. for 18 hours. The mixture was allowed to cool to room temperature, transferred to a separatory funnel and washed with saturated aqueous sodium bicarbonate. The organic phase was dried (Na₂SO₄), filtered, and the filtrate concentrated under reduced pressure. The residue was purified by flash chromatography on silica gel (elution with 85% hexanes:ethyl acetate then 50% hexanes:ethyl acetate) to provide 1.39 g (83% yield) of the title compound as a yellow oil. ¹H NMR (300 MHz, DMSO-d₆) δ 2.28 (s, 3H), 2.67 (m, 4H), 3.03 (m, 4H), 3.17 (s, 2H), 3.77 (s, 3H), 6.89 (m, 5H), 7.18 (dd, 1H, J=7.8, 7.8 Hz), 7.44 (m, 2H), 9.64 (br s, 1H); MS (DCI/NH₃) m/e 340 (M+H)⁺. HCl salt: white solid; mp 80° C. (dec); ¹H NMR (300 MHz, DMSO-d₆) δ 2.30 (s, 3H), 3.11 (br s, 2H), 3.46 (br s, 4H), 3.60 (br s, 2H), 3.80 (s, 3H), 4.25 (br s, 2H), 6.95 (m, 5H), 7.24 (dd, 1H, J=7.4, 7.4 Hz), 7.44 (m, 2H), 10.52 (br s, 0.5H), 10.82 (br s, 0.5H); Anal. calcd for C₂₀H₂₅N₃O₂.0.90 HCl: C, 64.53; H, 7.01; N, 11.29. Found: C, 64.38; H, 6.83; N, 11.17.

### EXAMPLE 2

### 2-[4-(2-cyanophenyl)-1-piperazinyl]-N-(3-methylphenyl)acetamide

The procedure described in Example 1B was followed, substituting 1-(2-cyanophenyl)piperazine (Chess) for 1-(2-methoxyphenyl)piperazine, to provide the title compound (92% yield) as a colorless oil. ¹H NMR (300 MHz, DMSO-d₆) δ 2.28 (s, 3H), 2.73 (m, 4H), 3.21 (s, 2H), 3.23 (m, 4H), 6.88 (br d, 1H, J=7.5 Hz), 7.10 (ddd, 1H, J=7.5, 7.5, 0.7 Hz), 7.19 (m, 2H), 7.44 (m, 2H), 7.61 (ddd, 1H, J=7.5, 7.5, 1.7 Hz), 7.70 (dd, 1H, J=7.8, 1.7 Hz), 9.68 (br s, 1H); MS (DCI/NH₃) m/e 335 (M+H)⁺.

Maleate salt: white solid, mp 168-170° C.; ¹H NMR (300 MHz, DMSO-d₆) δ 2.30 (s, 3H), 3.21 (br s, 4H), 3.37 (br s, 4H), 3.82 (br s, 2H), 6.13 (s, 2H), 6.93 (br d, 1H, J=7.4 Hz), 7.18 (m, 3H), 7.42 (m, 2H), 7.64 (ddd, 1H, J=7.5, 7.5, 1.4 Hz), 7.74 (dd, 1H, J=7.8, 1.7 Hz), 10.15 (br s, 1H); Anal. calcd for C₂₀H₂₂N₄O.1.0C₄H₄O₄: C, 63.99; H, 5.82; N, 12.44. Found: C, 63.80; H, 5.80; N, 12.21.

### EXAMPLE 3

### N-(3-methylphenyl)-2-[4-(2-pyrimidinyl]-1-piperazinyl]acetamide

The procedure described in Example 1B was followed, substituting 1-(2-pyrimidinyl)piperazine (EMKA-Chemie) for 1-(2-methoxyphenyl)piperazine, to provide the title compound (70% yield) as a white solid. mp 113-116° C.; ¹H NMR (300 MHz, DMSO-d₆) δ 2.28 (s, 3H), 2.57 (m, 4H), 3.17 (s, 2H), 3.80 (m, 4H), 6.62 (dd, 1H, J=4.8, 4.8 Hz), 6.88 (br d, 1H, J=7.4 Hz), 7.18 (dd, 1H, J=7.8, 7.8 Hz), 7.46 (m, 2H), 8.36 (d, 2H, J=4.7 Hz), 9.67 (br s, 1H); MS (DCI/NH₃) m/e 312 (M+H)⁺; Anal. calcd for C₁₇H₂₁N₅O: C, 65.57; H, 6.80; N, 22.49. Found: C, 65.39; H, 6.77; N, 22.56.

### EXAMPLE 4

### N-(3-methylphenyl)-2-[4-(2-pyridinyl)-1-piperazinyl]acetamide

The procedure described in Example 1B was followed, substituting 1-(2-pyridinyl)piperazine (Aldrich) for 1-(2-methoxyphenyl)piperazine, to provide the title compound (65% yield) as a white solid. mp 126-127° C.; ¹H NMR (300 MHz, DMSO-d₆) δ 2.27 (s, 3H), 2.60 (m, 4H), 3.17 (s, 2H), 3.55 (m, 4H), 6.63 (ddd, 1H, J=6.7, 4.7, 0.6 Hz), 6.82 (d, 1H, J=8.8 Hz), 6.88 (br d, 1H, J=7.8 Hz), 7.18 (dd, 1H, J=6.7, 4.7, 0.6 Hz), 7.46 (m, 2H), 7.52 (ddd, 1H, J=8.8, 7.1, 2.0 Hz), 8.11 (m, 1H), 9.67 (br s, 1H); MS (DCI/NH₃) m/e 311 (M+H)⁺; Anal. calcd for C₁₈H₂₂N₄O: C, 69.65; H, 7.14; N, 18.05. Found: C, 69.72; H, 7.09; N, 18.22.

### EXAMPLE 5

### 2-[4-(3-cyano-2-pyridinyl)-1-piperazinyl]-N-(3-methylphenyl)acetamide

The procedure described in Example 1B was followed, substituting 2-(1-piperazinyl)nicotinonitrile (Chess) for 1-(2-methoxyphenyl)piperazine, to provide the title compound (64% yield) as a white solid. mp 99-100° C.; ¹H NMR (300 MHz, DMSO-d₆) δ 2.28 (s, 3H), 2.68 (m, 4H), 3.19 (s, 2H), 3.68 (m, 4H), 6.88 (br d, 1H, J=7.8 Hz), 6.93 (dd, 1H, J=7.8, 4.8 Hz), 7.18 (dd, 1H, J=7.5, 7.5 Hz), 7.44 (br d, 1H, J=8.2 Hz), 7.47 (br s, 1H), 8.07 (dd, 1H, J=7.8, 2.0 Hz), 8.42 (dd, 1H, J=5.1, 2.0 Hz), 9.68 (br s, 1H); MS (DCI/NH₃) m/e 336 (M+H)⁺; Anal. calcd for C₁₉H₂₁N₅O: C, 68.04; H, 6.31; N, 20.88. Found: C, 68.19; H, 6.36; N, 21.15.

### EXAMPLE 6

### N-(3-methylphenyl)-2-[4-(2-methylphenly)-1-piperazinyl]acetamide

The procedure described in Example 1B was followed, substituting 1-(2-methylphenyl)piperazine (EMKA Chemie) for 1-(2-methoxyphenyl)piperazine, to provide the title compound (75% yield) as a white solid. mp 104-106° C.; ¹H NMR (300 MHz, DMSO-d₆) δ 2.24 (s, 3H), 2.28 (s, 3H), 2.69 (m, 4H), 2.91 (m, 4H), 3.19 (s, 2H), 6.88 (br d, 1H, J=7.4 Hz), 6.95 (dd, 1H, J=7.1, 7.1 Hz), 7.05 (m, 1H), 7.17 (m, 3H), 7.45 (m, 2H), 9.64 (br s, 1H); MS (DCI/NH₃) m/e 324 (M+H)⁺; Anal. calcd for C₂₀H₂₅N₃O: C, 74.27; H, 7.79; N, 12.99. Found: C, 74.34; H, 7.85; N, 12.91.

### EXAMPLE 7

### N-(3-methylphenyl)-2-[4-(2-nitrophenyl)-1-piperazinyl]acetamide

The procedure described in Example 1B was followed, substituting 1-(2-nitrophenyl)piperazine (EMKA Chemie) for 1-(2-methoxyphenyl)piperazine, to provide the title compound (91% yield) as an orange oil. ¹H NMR (300 MHz, DMSO-d₆) δ 2.28 (s, 3H), 2.66 (m, 4H), 3.07 (m, 4H), 3.18 (s, 2H), 6.88 (br d, 1H, J=7.8 Hz), 7.13 (ddd, 1H, J=8.5, 7.1, 1.0 Hz), 7.18 (dd, 1H, J=7.8, 7.8 Hz), 7.35 (dd, 1H, J=8.1, 1.0 Hz), 7.45 (m, 2H), 7.59 (ddd, 1H, J=8.1, 7.1, 1.3 Hz), 7.79 (dd, 1H, J=8.1, 1.7 Hz), 9.66 (br s, 1H); MS (DCI/NH₃) m/e 355 (M+H)⁺. maleate salt: yellow solid; mp 172-175° C.; Anal. calcd for C₁₉H₂₂N₄O₃.1.0 C₄H₄O₄: C, 58.72; H, 5.57; N, 11.91. Found: C, 58.38; H, 5.49; N, 11.64.

### EXAMPLE 8

### 2-[4-(3-cyano-2-pyridinyl)-1 piperazinyl]-N-(3-nitrophenyl)acetamide

2-(1-Piperazinyl)-3-pyridinecarbonitrile (640 mg, 3.40 mmol) and N,N-diisopropylethylamine (1.0 mL) in toluene (15 mL) at room temperature were treated with N-chloroacetyl-3-nitroaniline (Lancaster, 610 mg, 2.84 mmol) and the reaction was heated at 90° C. for 18 hours. The mixture was allowed to cool to room temperature, transferred to a separatory funnel and washed with saturated aqueous sodium bicarbonate. The organic phase was dried (Na₂SO₄), filtered, and the filtrate concentrated under reduced pressure. The residue was purified by flash chromatography on silica gel (elution with 85% hexanes:ethyl acetate) to provide 256 mg (25% yield) of the title compound as a light tan solid. mp 143-145° C.; ¹H NMR (300 MHz, DMSO-d₆) δ 2.69 (m, 4H), 3.27 (s, 2H), 3.70 (m, 4H), 6.93 (dd, 1H, J=7.4, 5.0 Hz), 7.61 (dd, 1H, J=8.1, 8.1 Hz), 7.93 (br d, 1H, J=8.2 Hz), 8.06 (dd, 2H, J=7.8, 7.8 Hz), 8.42 (m, 1H), 8.70 (br s, 1H), 10.28 (br s, 1H); MS (DCI/NH₃) m/e 367 (M+H)⁺; Anal. calcd for C₁₈H₁₈N₆O₃: C, 59.01; H, 4.95; N, 22.94. Found: C, 59.31; H, 5.25; N, 22.66.

### EXAMPLE 9

### 2-[4-(3-cyano-2-pyridinyl)-1-piperazinyl]-N-[3-(trifluoromethyl)phenyl]acetamide

The procedure described in Example 8 was followed, substituting N-chloroacetyl-3-(trifluoromethyl)aniline for N-chloroacetyl-3-nitroaniline, to provide the title compound (84% yield) as a yellow oil. ¹H NMR (300 MHz, DMSO-d₆) δ 2.69 (m, 4H), 3.25 (s, 2H), 3.69 (m, 4H), 6.93 (dd, 1H, J=7.8, 4.7 Hz), 7.41 (br d, 1H, J=7.8 Hz), 7.56 (dd, 1H, J=7.8, 7.8 Hz), 7.90 (br d, 1H, J=8.4 Hz), 8.07 (dd, 1H, J=7.8, 2.1 Hz), 8.15 (br s, 1H), 8.42 (dd, 1H, J=4.7, 1.7 Hz), 10.11 (br s, 1H); MS (DCI/NH₃) m/e 390 (M+H)⁺.

maleate salt: tan solid; mp 157-158° C.; ¹H NMR (300 MHz, DMSO-d₆) δ 3.07 (br s, 4H), 3.73 (br s, 2H), 3.79 (br s, 4H), 6.15 (s, 2H), 7.00 (dd, 1H, J=7.4, 4.7 Hz), 7.46 (br d, 1H, J=7.8 Hz), 7.59 (dd, 1H, J=7.8, 7.8 Hz), 7.85 (br d, 1H, J=8.2 Hz), 8.13 (m, 2H), 8.45 (dd, 1H, J=4.7, 2.0 Hz), 10.48 (br s, 1H); Anal. calcd for C₁₉H₁₈F₃N₅O.1.0 C₄H₄O₄: C, 54.56; H, 4.39; N, 13.86. Found: C, 54.30; H, 4.42; N, 13.42.

### EXAMPLE 10

### N-(3-methylphenyl)-2-(4-phenyl 1-piperazinyl)acetamide

The procedure described in Example 1B was followed, substituting 1-phenylpiperazine (Aldrich) for 1-(2-methoxyphenyl)piperazine, to provide the title compound (86% yield) as a white solid. mp 120-121° C.; ¹H NMR (300 MHz, DMSO-d₆) δ 2.27 (s, 3H), 2.66 (m, 4H), 3.17 (s, 2H), 3.20 (m, 4H), 6.77 (dd, 1H, J=7.1, 7.1 Hz), 6.88 (br d, 1H, J=7.5 Hz), 6.94 (d, 2H, J=7.8 Hz), 7.21 (m, 3H), 7.44 (m, 2H), 9.65 (br s, 1H); MS (DCI/NH₃) m/e 310 (M+H)⁺; Anal. calcd for C₁₉H₂₃N₃O: C, 73.76; H, 7.49; N, 13.58. Found: C, 73.73; H, 7.50; N, 13.64.

### EXAMPLE 11

### N-(3-cyanophenyl)-2-[4-(3-cyano-2-pyridinyl)-1-piperazinyl]acetamide

The procedure described in Example 8 was followed, substituting N-chloroacetyl-3-cyanoaniline (Maybridge) for N-chloroacetyl-3-nitroaniline, to provide the title compound (60% yield) as a colorless oil. ¹H NMR (300 MHz, DMSO-d₆) δ 2.68 (m, 4H), 3.25 (s, 2H), 3.69 (m, 4H), 6.92 (dd, 1H, J=7.5, 5.1 Hz), 7.52 (m, 2H), 7.94 (m, 1H), 8.07 (m, 1H), 8.15 (m, 1H), 8.41 (m, 1H), 10.10 (br s, 1H); MS (DCI/NH₃) m/e 347 (M+H)⁺.

maleate salt: white solid; mp 166-167° C.; ¹H NMR (300 MHz, DMSO-d₆) δ 3.04 (br s, 4H), 3.69 (br s, 2H), 3.78 (br s, 4H), 6.16 (s, 2H), 6.99 (dd, 1H, J=7.5, 4.6 Hz), 7.58 (m, 2H), 7.89 (m, 1H), 8.12 (m, 2H), 8.45 (dd, 1H, J=4.7, 2.0 Hz), 10.46 (br s, 1H); Anal. calcd for C₁₉H₁₈N₆O.1.0 C₄H₄O₄: C, 59.73; H, 4.79; N, 18.17. Found: C, 59.73; H, 4.81; N, 18.45.

### EXAMPLE 12

### N-(4-bromo-3-methylphenyl)-2-[4-(2-cyanophenyl)-1-piperazinyl]acetamide

### EXAMPLE 12A

### 2-bromo-N-(4-bromo-3-methylphenyl)acetamide

4-Bromo-3-methylaniline (10.08 g, 54.18 mmol) in 2N sodium hydroxide (200 mL) was treated with bromoacetyl chloride (5.00 mL, 60.8 mmol) as a solution in dichloromethane (200 mL) dropwise. After 15 minutes, the layers were separated. The organic phase was washed with 1N hydrochloric acid, dried (Na₂SO₄), filtered, and the filtrate concentrated under reduced pressure to provide 11.75 g (71%) of the title compound as a tan solid. ¹H NMR (300 MHz, CDCl₃) δ 2.39 (s, 3H), 4.01 (s, 2H), 7.23 (m, 1H), 7.44 (d, 1H, J=2.4 Hz), 7.49 (d, 1H, J=8.8 Hz), 8.07 (br s, 1H); MS (DCI/NH₃) m/e 306 (M+H)⁺.

### EXAMPLE 12B

### N-(4-bromo-3-methylphenyl)-2-[4-(2-cyanophenyl)-1-piperazinyl]acetamide

The product from Example 12A (3.51 g, 11.4 mmol) and N,N-diisopropylethylamine (2.50 mL) in toluene (50 mL) were treated with 1-(2-cyanophenyl)piperazine (Chess, 2.90 g, 15.5 mmol) and the reaction mixture was heated at 90° C. for 18 hours. The mixture was allowed to cool to room temperature and transferred to a separatory funnel with ethyl acetate and water. The organic phase was washed with saturated aqueous sodium bicarbonate, dried (Na₂SO₄), filtered, and the filtrate concentrated under reduced pressure. The residue was purified by flash chromatography on silica gel to provide 3.66 g (77%) of the title compound as a yellow solid. mp 143-145° C.; ¹H NMR (300 MHz, DMSO-d₆) δ 2.32 (s, 3H), 2.72 (m, 4H), 3.22 (m, 6H), 7.10 (ddd, 1H, J=7.4, 7.4, 0.6 Hz), 7.19 (d, 1H, 8.1 Hz), 7.49 (m, 2H), 7.61 (m, 1H), 7.65 (d, 1H, J=2.1 Hz), 7.70 (dd, 1H, J=7.8, 1.7 Hz), 9.82 (br s, 1H); MS (DCI/NH₃) m/e 413/415 (M+H)⁺; Anal. calcd for C₂₀H₂BrN₄O: C, 58.12; H, 5.12; N, 13.56. Found: C, 58.13; H, 5.07; N, 13.54.

### EXAMPLE 13

### 2-[4-(2-cyanophenyl)-1-piperazinyl]-N-phenylacetamide

The procedure described in Example 12B was followed, substituting 2-chloro-N-phenylacetamide (Maybridge) for the product from Example 12A, to provide the title compound (39% yield) as a yellow solid, mp 137-138° C.; ¹H NMR (300 MHz, DMSO-d₆) δ 2.73 (m, 4H), 3.22 (m, 6H), 7.08 (m, 2H), 7.19 (d, 1H, J=8.5 Hz), 7.30 (m, 2H), 7.63 (m, 2H), 7.70 (dd, 1H, J=7.8, 1.7 Hz), 9.76 (br s, 1H); MS (DCI/NH₃) m/e 321 (M+H)⁺; Anal. calcd for C₁₉H₂₀N₄O: C, 71.23; H, 6.29; N, 17.49. Found: C, 70.92; H, 6.34; N, 17.34.

### EXAMPLE 14

### 2-[4-(3-cyano-2-pyridinyl)-1-piperazinyl]-N-phenylacetamide

The procedure described in Example 8 was followed, substituting 2-chloro-N-phenylacetamide (Maybridge) for N-chloroacetyl-3-nitroaniline to provide the title compound (52% yield) as a white solid. mp 110-112° C.; ¹H NMR (300 MHz, DMSO-d₆) δ 2.68 (m, 4H), 3.21 (s, 2H), 3.68 (m, 4H), 6.93 (dd, 1H, J=7.8, 4.7 Hz), 7.06 (dd, 1H, J=7.8, 7.8 Hz), 7.31 (dd, 2H, J=7.8, 7.8 Hz), 7.64 (dd, 2H, J=8.8, 1.4 Hz), 8.07 (dd, 1H, J=7.8, 2.0 Hz), 8.42 (dd, 1H, J=4.8, 1.7 Hz), 9.76 (br s, 1H); MS (DCI/NH₃) m/e 322 (M+H)⁺; Anal. calcd for C18H₁₉N₅O: C, 67.27; H, 5.96; N, 21.79. Found: C, 67.21; H, 5.77; N, 21.59.

### EXAMPLE 15

### 2-[4-(3-cyano-2-pyridinyl)-1 piperazinyl]-N-(4-fluorophenyl)Acetamide

The procedure described in Example 8 was followed, substituting N-choroacetyl-4-fluoroaniline (Avocado) for N-chloroacetyl-3-nitroaniline, to provide the title compound (91% yield) as a white solid. mp 98-100° C.; ¹H NMR (300 MHz, DMSO-d₆) δ 2.68 (m, 4H), 3.20 (s, 2H), 3.68 (m, 4H), 6.92 (dd, 1H, J=7.5, 4.8 Hz), 7.15 (m, 2H), 7.67 (m, 2H), 8.07 (dd, 1H, J=7.8, 2.0 Hz), 8.41 (dd, 1H, J=4.8, 1.7 Hz), 9.83 (br s, 1H); MS (DCI/NH₃) m/e 340 (M+H)⁺; Anal. calcd for C₁₈H₁₈FN₅O: C, 63.71; H, 5.35; N, 20.64. Found: C, 63.57; H, 5.32; N, 20.79.

### EXAMPLE 16

### 2-[4-(3-cyano-2-pyridinyl)-1-piperazinyl]-N-(3,5-dimethylphenyl)acetamide

### EXAMPLE 16A

### 2-chloro-N-(3,5-dimethylphenyl)acetamide

3,5-Dimethylaniline (Acros, 10.50 mL, 84.05 mmol) in 2N sodium hydroxide (200 mL) was treated with chloroacetyl chloride (Acros, 10.00 mL, 125.7 mmol) as a solution in dichloromethane (200 mL) drop wise. After 18 hours, the layers were separated. The organic phase was washed with 1N hydrochloric acid, dried (Na₂SO₄), filtered, and the filtrate concentrated under reduced pressure to provide 15.64 g (94%) of the title compound as a white solid. ¹H NMR (300 MHz, DMSO-d₆) δ 2.24 (s, 6H), 4.21 (s, 2H), 6.73 (s, 1H), 7.20 (s, 2H), 10.11 (br s, 1H); MS (DCI/NH₃) m/e 198 (M+H)⁺.

### EXAMPLE 16B

### 2-[4-(3-cyano-2-pyridinyl)-1-piperazinyl]-N-(3,5-dimethylphenyl)acetamide

The procedure described in Example 8 was followed, substituting the product from Example 16A for N-chloroacetyl-3-nitroaniline to provide the title compound (63% yield) as a white solid. mp 139-140° C.; ¹H NMR (300 MHz, DMSO-d₆) δ 2.23 (s, 6H), 2.70 (m, 4H), 3.18 (s, 2H), 3.68 (m, 4H), 6.70 (br s, 1H), 6.93 (dd, 1H, J=7.8, 4.7 Hz), 7.28 (br s, 2H), 8.07 (dd, 1H, J=7.8, 2.0 Hz), 8.42 (dd, 1H, J=4.7, 2.0 Hz), 9.60 (br s, 1H); MS (DCI/NH₃) m/e 350 (M+H)⁺; Anal. calcd for C₂₀H₂₃N₅O: C, 68.74; H, 6.63; N, 20.04. Found: C, 68.56; H, 6.56; N, 20.05.

### EXAMPLE 17

### 2-[4-(3-cyano-2-pyridinyl)-1-piperazinyl]-N-(2.3-dimethylphenyl)acetamide

### EXAMPLE 17A

### 2-chloro-N-(2,3-dimethylphenyl)acetamide

The procedure described in Example 16A was followed, substituting 2,3-dimethylaniline for 3,5-dimethylaniline to provide the title compound (96% yield) as a white solid. ¹H NMR (300 MHz, DMSO-d₆) δ 2.07 (s, 3H), 2.24 (s, 3H), 4.28 (s, 2H), 7.07 (m, 3H), 9.70 (br s, 1H); MS (DCI/NH₃) m/e 198 (M+H)⁺.

### EXAMPLE 17B

### 2-[4-(3-cyano-2-pyridinyl)-1-piperazinyl]-N-(2,3-dimethylphenyl)acetamide

The procedure described in Example 8 was followed, substituting the product from Example 17A for N-chloroacetyl-3-nitroaniline, to provide the title compound (32% yield as a white solid. mp 124-126° C.; ¹H NMR (300 MHz, DMSO-d₆) δ 2.12 (s, 3H), 2.26 (s, 3H), 2.72 (m, 4H), 3.21 (s, 2H), 3.69 (m, 4H), 6.94 (dd, 1H, J=7.8, 4.8 Hz), 6.99 (br d, 1H, J=7.4 Hz), 7.07 (dd, 1H, J=7.4, 7.4 Hz), 7.45 (br d, 1H, J=7.8 Hz), 8.08 (dd, 1H, J=7.8, 2.0 Hz), 8.42 (dd, 1H, 4.8, 2.1 Hz), 9.42 (br s, 1H); MS (DCI/NH₃) m/e 350 (M+H)⁺; Anal. calcd for C₂₀H₂₃N₅0.0.10H₂O: C, 68.39; H, 6.66; N, 19.94. Found: C, 68.74; H, 6.58; N, 19.56.

### EXAMPLE 18

### 2-[4-(3-cyano-2-pyridinyl)-1-piperazinyl]-N-(2-methylphenyl)acetamide

### EXAMPLE 18A

### 2-chloro-N-(2-methylphenyl Acetamide

The procedure described in Example 16A was followed, substituting 2-methylaniline for 3,5-dimethylaniline to provide the title compound (90% yield) as a white solid. ¹H NMR (300 MHz, DMSO-d₆) δ 2.20 (s, 3H), 4.30 (s, 2H), 7.16 (m, 3H), 7.38 (d, 1H, J=7.8 Hz), 9.63 (br s, 1H); MS (DCI/NH₃) m/e 184 (M+H)⁺.

### EXAMPLE 18B

### 2-[4-(3-cyano-2-pyridinyl)-1-piperazinyl]-N-(2-methylhenyl)acetamide

The procedure described in Example 8 was followed, substituting the product from Example 18A for N-chloroacetyl-3-nitroaniline, to provide the title compound (58% yield) as a light yellow solid. mp 123-125° C.; ¹H NMR (300 MHz, DMSO-d₆) δ 2.25 (s, 3H), 2.73 (m, 4H), 3.22 (s, 2H), 3.69 (m, 4H), 6.94 (dd, 1H, J=7.8, 4.8 Hz), 7.06 (ddd, 1H, J=7.4, 7.4, 1.0 Hz), 7.17 (d, 1H, J=7.8 Hz), 7.21 (dd, 1H, 8.5, 8.5 Hz), 7.75 (d, 1H, J=7.8 Hz), 8.08 (dd, 1H, J=7.8, 1.7 Hz), 8.42 (dd, 1H, J=5.0, 1.7 Hz), 9.42 (br s, 1H); MS (DCI/NH₃) m/e 336 (M+H)⁺; Anal. calcd for C₁₉H₂₁N₅O_{0.20}H₂O: C, 67.32; H, 6.36; N, 20.66. Found: C, 67.29; H, 6.23; N, 20.66.

### EXAMPLE 19

### 2-[4-(3-cyano-2-pyridinyl)-1-piperazinyl]-N-(2,5-dimethylphenl)acetamide

### EXAMPLE 19A

### 2-chloro-N-(2,5-dimethylphenyl) Acetamide

The procedure described in Example 16A was followed, substituting 2,5-dimethylaniline for 3,5-dimethylaniline, to provide the title compound (89% yield) as a white solid. ¹H NMR (300 MHz, DMSO-d₆) δ 2.14 (s, 3H), 2.24 (s, 3H), 4.28 (s, 2H), 6.93 (d, 1H, J=7.8 Hz), 7.10 (d, 1H, J=7.8 Hz), 7.20 (s, 1H), 9.57 (br s, 1H); MS (DCI/NH₃) m/e 198 (M+H)⁺.

### EXAMPLE 19B

### 2-[4-(3-cyano-2-pyridinyl)-1-piperazinyl]-N-(2,5-dimethylphenyl)acetamide

The procedure described in Example 8 was followed, substituting Example 19A for N-chloroacetyl-3-nitroaniline, to provide the title compound (34% yield) as a white solid. mp 106-108° C.; ¹H NMR (300 MHz, DMSO-d₆) δ 2.19 (s, 3H), 2.26 (s, 3H), 2.72 (m, 4H), 3.20 (s, 2H), 3.69 (m, 4H), 6.87 (d, 1H, J=7.4 Hz), 6.94 (dd, 1H, J=7.5, 4.8 Hz), 7.10 (d, 1H, J=7.8 Hz), 7.59 (br s, 1H), 8.08 (dd, 1H, J=7.8, 2.0 Hz), 8.42 (dd, 1H, J=4.7, 2.0 Hz), 9.35 (br s, 1H); MS (DCI/NH₃) m/e 350 (M+H)⁺; Anal. calcd for C₂₀H₂₃N₅O.0.20H₂O: C, 68.04; H, 6.68; N, 19.84. Found: C, 67.89; H, 6.54; N, 19.88.

### EXAMPLE 20

### N-(3-chlorophenyl)-2-[4-(3-cyano-2-pyridinyl-1-piperazinyl]acetamide

The procedure described in Example 8 was followed, substituting 3-chloro-N-(chloroacetyl)aniline (Maybridge) for N-chloroacetyl-3-nitroaniline to provide the title compound (79% yield) as a light tan solid. mp 108-109° C.; ¹H NMR (300 MHz, DMSO-d₆) δ 2.68 (m, 4H), 3.23 (s, 2H), 3.69 (m, 4H), 6.93 (dd, 11H, J=7.8, 4.8 Hz), 7.12 (m, 1H), 7.34 (dd, 11H, J=8.1, 8.1 Hz), 7.57 (m, 1H), 7.86 (m, 1H), 8.07 (dd, 1H, J=7.8, 2.0 Hz), 8.42 (dd, 1H, J=4.7, 2.0 Hz), 9.96 (br s, 1H); MS (DCI/NH₃) m/e 356 (M+H)⁺; Anal. calcd for C₁₈H₁₈ClN₅O: C, 60.76; H, 5.10; N, 19.68. Found: C, 60.71; H, 5.09; N, 19.58.

### EXAMPLE 21

### N-(3-chloro-4-fluorophenyl)-2-[4-(3-cyano-2-pyridinyl)-1-piperazinyl] acetamide

The procedure described in Example 8 was followed, substituting 3-chloro-N-(chloroacetyl)-4-fluoroaniline (Maybridge) for N-chloroacetyl-3-nitroaniline, to provide the title compound (39% yield) as a light tan solid. mp 137-140° C.; ¹H NMR (300 MHz, DMSO-d₆) δ 2.67 (m, 4H), 3.22 (s, 2H), 3.69 (m, 4H), 6.93 (dd, 1H, J=7.5, 4.8 Hz), 7.37 (dd, 1H, J=9.1, 9.1 Hz), 7.61 (ddd, 1H, J=9.2, 4.5, 2.8 Hz), 7.98 (dd, 1H, J=7.2, 2.8 Hz), 8.07 (dd, 1H, J=7.8, 2.0 Hz), 8.41 (dd, 1H, J=4.8, 1.7 Hz), 9.98 (br s, 1H); MS (DCI/NH₃) m/e 374 (M+H)⁺; Anal. calcd for C₁₈H₁₇ClFN₅O: C, 57.84; H, 4.58; N, 18.73. Found: C, 57.98; H, 4.42; N, 18.65.

### EXAMPLE 22

### 2-[4-(3-cyano-2-pyridinyl)-1-piperazinyl]-N-(3,4,5-trimethoxyphenyl)acetamide

### EXAMPLE 22A

### 2-chloro-N-(3,4,5-trimethoxyphenyl)acetamide

3,4,5-Trimethoxyaniline (Aldrich, 4.06 g, 22.2 mmol) and chloroacetyl chloride (2.60 mL, 32.7 mmol) in toluene (50 mL) were heated at 100° C. for 24 hours. The mixture was allowed to cool to room temperature and the volatiles were removed under reduced pressure. The residue was taken up in toluene and concentrated (3×) to remove traces of starting acid chloride and placed under high vacuum to provide 5.26 g (91%) of the title compound as a light brown solid. ¹H NMR (500 MHz, DMSO-d₆) δ 3.62 (s, 3H), 3.74 (s, 6H), 4.21 (s, 2H), 6.96 (s, 2H), 10.19 (br s, 1H); MS (DCI/NH₃) m/e 260 (M+H)⁺.

### EXAMPLE 22B

### 2-[4-(3-cyano-2-pyridinyl)-1-piperazinyl]-N-(3,4,5-trimethoxyphenyl)acetamide

The procedure described in Example 8 was followed, substituting Example 22A for N-chloroacetyl-3-nitroaniline, to provide the title compound (69% yield) as a light tan solid. mp 123-124° C.; ¹H NMR (300 MHz, DMSO-d₆) δ 2.68 (m, 4H), 3.19 (s, 2H), 3.61 (s, 3H), 3.69 (m, 4H), 3.74 (s, 6H), 6.93 (dd, 11H, J=7.8, 4.7 Hz), 7.07 (s, 2H), 8.07 (dd, 11H, J=7.4, 1.7 Hz), 8.42 (dd, 11H, J=4.8, 2.1 Hz), 9.67 (br s, 1H); MS (DCI/NH₃) m/e 412 (M+H)⁺; Anal. calcd for C₂₁H₂₅N₅O: C, 61.30; H, 6.12; N, 17.02. Found: C, 61.27; H, 6.08; N, 16.95.

### EXAMPLE 23

### 2-[4-(3-cyano-2-pyridinyl)-1-piperazinyl]-N-[4-fluoro-3-(trifluoromethyl)phenyl]acetamide

### EXAMPLE 23A

### 2-chloro-N-(4-fluoro-3-trifluoromethylphenyl) acetamide

The procedure described in Example 16A was followed, substituting 4-fluoro-3-(trifluoromethyl)aniline (Acros) for 3,5-dimethylaniline, to provide the title compound (79% yield) as a white solid. ¹H NMR (300 MHz, DMSO-d₆) δ 4.29 (s, 2H), 7.50 (dd, 1H, J=9.8, 9.8 Hz), 7.85 (m,1H), 8.08 (dd, 1H, J=6.5, 2.7 Hz), 10.64 (br s, 1H).

### EXAMPLE 23B

### 2-[4-(3-cyano-2-pyridinyl)-1-piperazinyl]-N-[4-fluoro-3-(trifluoromethyl)phenyl]acetamide

The procedure described in Example 8 was followed, substituting the product from Example 23A for N-chloroacetyl-3-nitroaniline, to provide the title compound (47% yield) as a white solid. mp 120-122° C.; ¹H NMR (300 MHz, DMSO-d₆) δ 2.68 (m, 4H), 3.24 (s, 2H), 3.69 (m, 4H), 6.93 (dd, 1H, J=8.1, 5.1 Hz), 7.48 (dd, 1H, J=10.2, 10.2 Hz), 7.97 (m, 1H), 8.07 (dd, 1H, J=7.1, 2.0 Hz), 8.16 (dd, 1H, J=6.8, 2.7 Hz), 8.42 (dd, 1H, J=4.8, 2.0 Hz), 10.1 (br s, 1H); MS (DCI/NH₃) m/e 408 (M+H)⁺; Anal. calcd for C₁₉H₁₇F₄N₅O: C, 56.02; H, 4.21; N, 17.19. Found: C, 55.94; H, 4.14; N, 17.31.

### EXAMPLE 24

### 2-[4-(3-cyano-2-pyridinyl)-1-piperazinyl]-N-[3-fluoro-5-(trifluoromethyl phenyl Acetamide

### EXAMPLE 24A

### 2-chloro-N-(3-fluoro-5-trifluoromethylphenyl) Acetamide

The procedure described in Example 16A was followed, substituting 3-fluoro-5-(trifluoromethyl)aniline (Oakwood) for 3,5-dimethylaniline, to provide the title compound (79% yield) as a white solid. ¹H NMR (300 MHz, CDCl₃) δ 4.22 (s, 2H), 7.14 (m, 1H), 7.49 (br s, 1H), 7.76 (ddd, 1H, J=10.1, 2.0, 2.0 Hz), 8.37 (br s, 1H).

### EXAMPLE 24B

### 2-[4-(3-cyano-2-pyridinyl)-1-pilerazinyl]-N-[3-fluoro-5-(trifluoromethyl)phenyl]Acetamide

The procedure described in Example 8 was followed, substituting the product from Example 24A for N-chloroacetyl-3-nitroaniline, to provide the title compound (41% yield) as a white solid. mp 108-110° C.; ¹H NMR (300 MHz, DMSO-d₆) δ 2.68 (m, 4H), 3.26 (s, 2H), 3.70 (m, 4H), 6.93 (dd, 1H, J=7.8, 4.7 Hz), 7.36 (m, 1H), 7.93 (m, 2H), 8.08 (dd, 1H, 7.7, 2.0 Hz), 8.42 (dd, 1H, J=4.7, 2.0 Hz), 10.28 (br s, 1H); MS (DCI/NH₃) m/e 408 (M+H)⁺; Anal. calcd for C₁₉H₁₇F₄N₅O: C, 56.02; H, 4.21; N, 17.19. Found: C, 56.17; H, 4.11; N, 17.43.

### EXAMPLE 25

### 2-[4-(3-cyano-2-pyridinyl)-1-piperaziny]-1-N-[2-fluoro-5-(trifluoromethyl)phenyl]acetamide

### EXAMPLE 25A

### 2-chloro-N-(2-fluoro-5-(trifluoromethyl)phenyl)acetamide

The procedure described in Example 22A was followed, substituting 2-fluoro-5-(trifluoromethyl)aniline (Acros) for 3,4,5-trimethoxyaniline, to provide the title compound (66% yield) as a tan solid. ¹H NMR (300 MHz, DMSO-d₆) δ 4.40 (s, 2H), 7.58 (m, 2H), 8.38 (dd, 1H, J=7.4, 2.0 Hz), 10.42 (br s, 1H).

### EXAMPLE 25B

### 2-[4-(3-cyano-2-pyridinyl)-1-piperazinyl]-N-[2-fluoro-5-(trifluoromethyl)phenyl]acetamide

The procedure described in Example 8 was followed, substituting the product from Example 25A for N-chloroacetyl-3-nitroaniline to provide the title compound (61% yield) as a white solid. mp 130-133° C.; ¹H NMR (300 MHz, DMSO-d₆) δ 2.73 (m, 4H), 3.31 (s, 2H), 3.67 (m, 4H), 6.94 (dd, 1H, J=7.8, 5.1 Hz), 7.56 (m, 2H), 8.08 (dd, 1H, J=7.5, 2.1 Hz), 8.42 (m, 2H), 9.91 (br s, 1H); MS (DCI/NH₃) m/e 408 (M+H)⁺; Anal. calcd for C₁₉H₁₇F₄N₅O: C, 56.02; H, 4.21; N, 17.19. Found: C, 55.88; H, 4.14; N, 17.15.

### EXAMPLE 26

### 2-[4-(3-cyano-2-pyridinyl)-1-piperazinyl]-N-[2-fluoro-3-(trifluoromethyl)phenyl]acetamide

### EXAMPLE 26A

### 2-chloro-N-(2-fluoro-3-trifluoromethyl)phenyl]acetamide

The procedure described in Example 22A was followed, substituting 2-fluoro-3-(trifluoromethyl)aniline (Acros) for 3,4,5-trimethoxyaniline, to provide the title compound (72% yield) as a white solid. ¹H NMR (300 MHz, DMSO-d₆) δ 4.39 (m, 2H), 7.41 (dd, 1H, J=8.2, 8.2 Hz), 7.57 (dd, 1H, J=6.5 Hz), 8.18 (dd, 1H, J=7.1 Hz), 10.37 (br s, 1H).

### EXAMPLE 26B

### 2-[⁴-(3-cyano-2-pyridinyl)-1-piperazinyl]-N-[2-fluoro-3-(trifluoromethyl)phenyl]acetamide

The procedure described in Example 8 was followed, substituting the product from Example 26A for N-chloroacetyl-3-nitroaniline, to provide the title compound (66% yield) as a white solid. mp 118-121° C.; ¹H NMR (300 MHz, DMSO-d₆) δ 2.72 (m, 4H), 3.30 (s, 2H), 3.68 (m, 4H), 6.94 (dd, 1H, J=7.5, 4.8 Hz), 7.40 (dd, 1H, J=8.1, 8.1 Hz), 7.54 (m, 1H), 8.08 (dd, 1H, J=7.8, 2.0 Hz), 8.23 (dd, 1H, J=8.5, 8.5 Hz), 8.42 (dd, 1H, J=5.1, 2.1 Hz), 9.90 (br s, 1H); MS (DCI/NH₃) m/e 408 (M+H)⁺; Anal. calcd for C₁₉H₁₇F₄N₅O: C, 56.02; H, 4.21; N, 17.19. Found: C, 55.82; H, 4.20; N, 17.18.

### EXAMPLE 27

### 2-[4-(3-cyano-2-pyridinyl)-1-piperazinyl]-N-(4-fluoro-3-methylphenyl)acetamide

### EXAMPLE 27A

### 2-chloro-N-(4-fluoro-3-methylphenyl)acetamide

The procedure described in Example 16A was followed, substituting 4-fluoro-3-methylaniline (Lancaster) for 3,5-dimethylaniline, to provide the title compound (83% yield) as a white solid. ¹H NMR (300 MHz, DMSO-d₆) δ 2.21 (d, 3H, J=2.1 Hz), 4.23 (s, 2H), 7.10 (dd, 1H, J=9.2, 9.2 Hz), 7.40 (m, 1H), 7.49 (dd, 11H, J=7.1, 2.4 Hz), 10.25 (br s, 1H); MS (DCI/NH₃) m/e 219 (M+NH₄)⁺.

### EXAMPLE 27B

### 2-[4-(3-cyano-2-pyridinyl)-1-piperazinyl]-N-(4-fluoro-3-methylphenyl)acetamide

The procedure described in Example 8 was followed, substituting the product from Example 27A for N-chloroacetyl-3-nitroaniline, to provide the title compound (67% yield) as a light tan solid. mp 111-113° C.; ¹H NMR (300 MHz, DMSO-d₆) δ 2.21 (d, 3H, J=1.7 Hz), 2.67 (m, 4H), 3.19 (s, 2H), 3.68 (m, 4H), 6.93 (dd, 1H, J=7.6, 4.8 Hz), 7.07 (dd, 1H, J=9.2, 9.2 Hz), 7.49 (m, 1H), 7.55 (m, 1H), 8.07 (dd, 1H, J=7.5, 2.1 Hz), 8.41 (dd, 1H, J=4.7, 2.0 Hz), 9.74 (br s, 1H); MS (DCI/NH₃) m/e 354 (M+H)⁺; Anal. calcd for C₁₉H₂₀FN₅O: C, 64.57; H, 5.70; N, 19.82. Found: C, 64.34; H, 5.73; N, 19.83.

### EXAMPLE 28

### 2-[4-(3-cyano-2-pyridinyl)-1-piperazinyl]-N-(2-fluorophenyl)acetamide

### EXAMPLE 28A

### 2-chloro-N-(2-fluorophenyl)acetamide

The procedure described in Example 22A was followed, substituting 2-fluoroaniline (Aldrich) for 3,4,5-trimethoxyaniline, to provide the title compound (88% yield) as a white solid. ¹H NMR (300 MHz, DMSO-d₆) δ 4.35 (s, 2H), 7.23 (m, 3H), 7.87 (m, 1H), 10.17 (br s, 1H); MS (DCI/NH₃) m/e 188 (M+H)⁺.

### EXAMPLE 28B

### 2-[4-(3-cyano-2-pyridinyl)-1-piperazinyl]-N-(2-fluorophenyl Acetamide

The procedure described in Example 8 was followed, substituting the product from Example 28A for N-chloroacetyl-3-nitroaniline, to provide the title compound (42% yield) as a white solid. mp 78-79° C.; ¹H NMR (300 MHz, DMSO-d₆) δ 2.71 (m, 4H), 3.27 (s, 2H), 3.67 (m, 4H), 6.94 (dd, 1H, J=7.8, 4.8 Hz), 7.18 (m, 2H), 7.26 (m, 1H), 7.98 (m, 1H), 8.08 (dd, 1H, J=7.8, 2.0 Hz), 8.42 (dd, 1H, J=5.1, 2.1 Hz), 9.65 (br s, 1H); MS (DCI/NH₃) m/e 340 (M+H)⁺; Anal. calcd for C₁₈H₁₈FN₅O: C, 63.70; H, 5.35; N, 20.64. Found: C, 63.48; H, 5.32; N, 20.54.

### EXAMPLE 29

### 2-[4-(3-cyano-2-pyridinyl)-1-piperazinyl]-N-(2-methoxyphenyl)acetamide

### EXAMPLE 29A

### 2-chloro-N-(2-methoxyphenyl)acetamide

The procedure described in Example 22A was followed, substituting 2-methoxyaniline (Acros) for 3,4,5-trimethoxyaniline, to provide the title compound (83% yield) as a brown solid. ¹H NMR (300 MHz, DMSO-d₆) δ 3.85 (s, 3H), 4.38 (s, 2H), 6.92 (m, 1H), 7.08 (m, 2H), 7.91 (d, 1H, J=7.8 Hz), 9.48 (br s, 1H); MS (DCI/NH₃) m/e 200 (M+H)⁺.

### EXAMPLE 29B

### 2-[4-(3-cyano-2-pyridinyl)-1-piperazinyl]-N-(2-methoxyphenyl)acetamide

The procedure described in Example 8 was followed, substituting the product from Example 29A for N-chloroacetyl-3-nitroaniline, to provide the title compound (34% yield) as a white solid. mp 174-175° C.; ¹H NMR (300 MHz, DMSO-d₆) δ 2.71 (m, 4H), 3.22 (s, 2H), 3.70 (m, 4H), 3.88 (s, 3H), 6.96 (m, 2H), 7.07 (m, 2H), 8.10 (dd, 1H, J=7.8, 2.1 Hz), 8.21 (d, 1H, J=7.8 Hz), 8.44 (dd, 1H, 4.7, 1.7 Hz), 9.73 (br s, 1H); MS (DCI/NH₃) m/e 352 (M+H)⁺; Anal. calcd for C₁₉H₂₁N₅O₂: C, 64.94; H, 6.02; N, 19.93. Found: C, 64.70; H, 5.95; N, 19.71.

### EXAMPLE 30

### 2-[4-(3-cyano-2-pyridinyl)-1-piperazinyl]-N-(2-nitrophenyl)acetamide

### EXAMPLE 30A

### 2-chloro-N-(2-nitrophenyl)acetamide

The procedure described in Example 22A was followed, substituting 2-nitroaniline (Aldrich) for 3,4,5-trimethoxyaniline to provide the title compound (94% yield) as a yellow solid. ¹H NMR (300 MHz, DMSO-d₆) δ 4.38 (s, 2H), 7.41 (ddd, 1H, J=8.1, 7.1, 1.7 Hz), 7.77 (m, 2H), 8.03 (dd, 1H, J=8.2, 1.4 Hz), 10.68 (br s, 1H); MS (DCI/NH₃) m/e 232 (N+NH₄)⁺.

### EXAMPLE 30B

### 2-[4-(3-cyano-2-pyridinyl)-1-piperazinyl]-N-(2-nitrophenyl)acetamide

The procedure described in Example 8 was followed, substituting the product from Example 30A for N-chloroacetyl-3-nitroaniline, to provide the title compound (39% yield) as a yellow solid. mp 134-136° C.; ¹H NMR (300 MHz, DMSO-d₆) δ 2.75 (m, 4H), 3.29 (s, 2H), 3.72 (m, 4H), 6.96 (dd, 11H, J=7.8, 4.8 Hz), 7.32 (ddd, 1H, J=8.5, 7.1, 1.4 Hz), 7.78 (ddd, 1H, J=8.8, 7.4, 1.6 Hz), 8.10 (dd, 1H, J=7.8, 2.1 Hz), 8.20 (dd, 1H, J=8.2, 1.4 Hz), 8.44 (dd, 1H, J=5.1, 2.0 Hz), 8.61 (dd, 1H, J=8.5, 1.4 Hz), 11.55 (br s, 1H); MS (DCI/NH₃) m/e 367 (M+H)⁺; Anal. calcd for C₁₈H₁₈N₆O: C, 59.01; H, 4.95; N, 22.94. Found: C, 58.87; H, 5.01; N, 23.08.

### EXAMPLE 31

### 2-[4-(3-cyano-2-pyridinyl)-1-piperazinyl]-N-[2-(trifluoromethyl)phenyl]acetamide

The procedure described in Example 8 was followed, substituting N-chloroacetyl-2-(trifluoromethyl)aniline (Apollo) for N-chloroacetyl-3-nitroaniline to provide the title compound (47% yield) as a colorless oil. ¹H NMR (300 MHz, DMSO-d₆) δ 2.74 (m, 4H), 3.27 (s, 2H), 3.65 (m, 4H), 6.97 (dd, 1H, J=7.5, 4.8 Hz), 7.36 (dd, 1H, J=7.8, 7.8 Hz), 7.69 (d, 1H, J=7.5 Hz), 7.73 (dd, 1H, J=8.1, 8.1 Hz), 8.10 (dd, 1H, J=8.1, 2.0 Hz), 8.21 (d, 1H, J=8.5 Hz), 8.44 (dd, 1H, J=4.7, 2.3 Hz), 9.89 (br s, 1H); MS (DCI/NH₃) m/e 390 (M+H)⁺. Maleate salt: white solid, mp 143-145° C.; Anal. calcd for C₁₉H₁₈F₃N₅O_{1.0} C₄H₄O₄: C, 54.65; H, 4.39; N, 13.86. Found: C, 54.61; H, 4.32; N, 13.83.

### EXAMPLE 32

### N-phenyl-2-[4-(2-pyridinyl)-1-piperazinyl]acetamide

N-Chloroacetylaniline (0.5 g, 2.95 mmol), 1-(2-pyridinyl)piperazine (0.72 g, 4.42 mmol), and N,N-diisopropylethylamine (1.03 mL, 5.9 mmol) were combined in toluene and heated at reflux overnight. The mixture was allowed to cool to room temperature, filtered, and the filtrate concentrated under reduced pressure. The residue was purified by flash column chromatography on silica gel (elution with 60% ethyl acetate:hexanes) to provide 400 mg (46% yield) of the title compound as a hygroscopic white solid. ¹H NMR (300 MHz, CDCl₃) δ 2.60 (m, 4H), 3.18 (s, 2H), 3.55 (m, 4H), 6.65 (dd, 1H, J=12, 6 Hz), 6.85 (d, 1H, J=9 Hz), 7.05 (t, 1H, J=6 Hz), 7.3 (t, 2H, J=9 Hz), 7.51 (ddd, 1H, J=9, 7.5, 3 Hz) 7.68 (d, 2H, J=9 Hz) 8.1 (dd, 1H, J=6, 3 Hz) 9.75 (br s, 1H); MS (DCI/NH₃) m/e 297 (M+H)⁺; Anal. calcd for C₁₇H₂₀N₄O: C, 68.89; H, 6.80; N, 18.90. Found: C, 68.97; H, 6.87; N, 19.01.

### EXAMPLE 33

### N-(3-methylphenyl)-2-[4-(1,3-thiazol-2-yl)-1-piperazinyl]acetamide

### EXAMPLE 33A

### 2-chloro-N-(3-methylphenyl)acetamide

3-Methylaniline (1 g, 9.3 mmol) in 2N aqueous sodium hydroxide (30 mL) was treated with chloroacetyl chloride (0.82 mL, 10.27 mmol) dropwise at room temperature as a solution in dichloromethane. After 18 hours, the reaction mixture was quenched with water and the layers separated. The organic phase was washed with an aqueous solution of 1N HCl and dried over MgSO₄, filtered and the filtrate concentrated under reduced pressure to provide 1.3 g (76%) of the title compound as a white solid. ¹H NMR (300 MHz, CDCl₃) δ 2.35 (s, 3H), 4.20 (s, 2H), 7.00 (s, 1H), 7.22 (m, 1H), 7.35-7.45 (m, 2H), 8.15 (br s, 1H); MS (DCI/NH₃) m/e 201 (M+NH₄)⁺.

### EXAMPLE 33B

### 1-(13-thiazol-2-yl)piperazine

The procedure described in J. Med. Chem 1996, 39(7), 1431 was followed to provide the title compound. A mixture of 2-bromothiazole (3 g, 18.3 mmol) and piperazine (3.15 g, 36.6 mmol) was refluxed in n-butanol for 18 hours. The reaction mixture was allowed to cool to room temperature and concentrated under reduced pressure. The residue was treated with an aqueous solution of 10% K₂CO₃ and extracted with ethyl acetate. The organic phase was dried over MgSO₄, filtered, and the filtrate concentrated under reduced pressure to provide 2.7 g (87%) of the title compound as brown oil used directly in the next step without further purification. ¹H NMR (300 MHz, CDCl₃) δ 2.99 (m, 4H), 3.47 (m, 4H), 6.57 (d, 1H, J=4.5 Hz), 7.20 (d, 1H, J=4.5 Hz); MS (DCI/NH₃) m/e 170 (M+H)⁺.

### EXAMPLE 33C

### N-(3-methylphenyl-2-[4-(1,3-thiazol-2-yl)-1-piperazinyl]acetamide

The product from Example 33A (0.2 g, 1.18 mmol), the product from Example 33B (0.25 g, 1.48 mmol) and N,N-diisopropylethylamine (0.41 mL, 2.3 mmol) were combined in toluene (25 mL) and heated at reflux overnight. The reaction was allowed to cool to room temperature, filtered, and the filtrate was concentrated under reduced pressure. The residue was purified by column chromatography on silica gel (elution with 50% ethyl acetate:hexanes) to provide 0.08 g (22%) of the desired material as a white solid. mp 151-153° C.; ¹H NMR (300 MHz, DMSO-d₆) δ 2.28 (s, 3H), 2.65 (m, 4H), 3.20 (s, 2H), 3.48 (m, 4H), 6.85 (m, 2H), 7.18 (m, 2H), 7.48 (m, 2H), 9.65 (s, 1H); MS (DCI/NH₃) m/e 317 (M+H)⁺; Anal calcd for C₁₆H₂₀N₄OS: C, 60.73; H, 6.37; N, 17.71. Found: C, 60.66; H, 6.24; N, 17.35.

### EXAMPLE 34

### 2-[4-(3-cyano-2-pyridinyl)-1-piperazinyl]-N-(4-methylphenyl)acetamide

### EXAMPLE 34A

### 2-chloro-N-(4-methylphenyl)acetamide

The procedure described in Example 33A was followed, substituting 4-methylaniline for 3-methylaniline, to provide a white solid. ¹H NMR (300 MHz, CDCl₃) δ 2.30 (s, 3H), 4.20 (s, 2H), 7.15 (d, 2H, J=9 Hz), 7.41 (m, 2H), 8.15 (br s, 1H); MS (DCI/NH₃) m/e 201 (M+NH₄)+.

### EXAMPLE 34B

### 2-[4-(3-cyano-2-pyridinyl)-1-piperazinyl]-N-(4-methylphenyl)acetamide

The product from Example 34A (0.4 g, 2.18 mmol), 1-(2-cyanopyridinyl)piperazine (0.62 g, 3.3 mmol) and N,N-diisopropylethylamine (0.76 mL, 4.36 mmol) in toluene (50 ML) were heated at reflux. After 18 hours, the reaction mixture was allowed to cool to room temperature, filtered, and the filtrate was concentrated under reduced pressure. The residue purified by column chromatography on silica gel (elution with 60% ethyl acetate:hexanes) to provide 0.51 g (70%) of the desired material as a yellow oil. ¹H NMR (300 MHz, CDCl₃) δ 2.30 (s, 3H), 2.80 (m, 4H), 3.25 (s, 2H), 3.80 (m, 4H), 6.80 (dd, 1H, J=12, 6 Hz), 7.12 (d, 2H, J=9 Hz), 7.48 (d, 2H, J=9 Hz), 7.80 (dd, 1H, J=9, 3 Hz), 8.38 (dd, 1H, J=6, 3 Hz), 9.10 (br, s, 1H); MS (DCI/NH₃) m/e 336 (M+H)⁺; maleate salt: obtained as an off-white powder; mp 156-158° C.; Anal. calcd for C₂₃H₂₅N₅O₅.0.20H₂O: C, 60.70; H, 5.63; N, 15.39. Found: C, 60.33; H, 5.55; N, 15.10.

### EXAMPLE 35

### 2-[4-(2-methoxyphenyl)-1-piperidinyl]-N-(3-methylphenyl)acetamide

4-(2-Methoxyphenyl)piperidine (200 mg, 1 mmol), the product from Example 1A (228 mg, 1 mmol) and N,N-diisopropylethylamine (0.185 mL, 1.1 mmol) in toluene (8 mL) were stirred at 60° C. for 18 hours. The reaction mixture was poured into water (30 mL) and extracted with ethyl acetate (30 mL). The organic layer was washed with brine (2×30 mL), dried over MgSO₄, filtered and the filtrate concentrated under reduced pressure. The residue was purified by flash chromatography on silica gel (elution with dichloromethane:methanol, 9.5:0.5) to provide the title compound 177 mg (52.3%). ¹H NMR (300 MHz, DMSO-d₆) δ 1.71 (m, 4H), 2.28 (m, 5H), 2.89 (m, 1H), 2.96 (m, 2H), 3.13 (s, 2H), 3.78 (s, 3H), 6.91 (m, 3H), 7.20 (m, 3H), 7.45 (m, 2H), 8.69 (s, 1H); MS (DCI/NH₃) m/e 339 (M+H)⁺. Anal. calcd for C₂₁H₂₆N₂O₂: C, 74.52; H, 7.74; N, 8.28. Found: C, 74.23, H, 7.71, N, 8.26.

### EXAMPLE 36

### N-(3-methylphenyl)[-2-[4-(2-pyridinyl]-1-piperidinyl] acetamide

### EXAMPLE 36A

### benzyl 4-hydroxy-4-(2-pyridinyl)-1-piperidinecarboxylate

2-Bromopyridine (0.470 mL, 5 mmol) in THF (20 mL) was treated with n-BuLi 1.6M in hexanes (5.2 ml, 5.2 mmol) dropwise at −60° C. After stirring at −60° C. for 30 minutes, the reaction mixture and treated with benzyl 4-oxo-1-piperidinecarboxylate (1.14 g, 4.9 mmol) in THF (10 mL) slowly. After stirring an additional 15 minutes at −60° C., the reaction mixture was quenched with a saturated aqueous solution of NH₄Cl, allowed to warm to room temperature and was extracted into dichloromethane. The organics were combined, dried on MgSO₄, filtered, and the filtrate was concentrated under reduced pressure. The residue was purified by flash chromatography on silica gel (elution with hexanes:ethyl acetate, 1: 1) to provide the title compound (400 mg, 27% yield). ¹H NMR (300 MHz, DMSO-d₆) δ 1.54 (m, 2H), 2.05 (m, 2H), 3.25 (m, 2H), 3.95 (m, 2H), 5.11 (s, 2H), 5.35 (s, 1H), 7.25 (m, 1H), 7.35 (m, 5H), 7.68 (m, 1H), 7.79 (m, 1H), 8.5 (m, 1H); MS (DCI/NH₃) m/e 313 (M+H)⁺.

### EXAMPLE 36B

### benzyl 3′,6′-dihydro-2,4′-bipyridine-1′(2′H)-carboxylate

The product from Example 36A (400 mg, 1.28 mmol) in thionyl chloride (6 mL) was refluxed for 3 hours, allowed to cool to room temperature, and concentrated under reduced pressure. The residue was treated with ice and 40% aqueous sodium hydroxide then extracted into dichloromethane, washed with brine, dried (Na₂SO₄), filtered, and the filtrate concentrated under reduced pressure to provide the title compound (332 mg).

### EXAMPLE 36C

### 2-(4-piperidinyl)pyridine

The product from Example 36B was treated with 10% Pd/C (250 mg) at 60 psi and 50° C. for 40 hours to provide the title compound (150 mg, 88% yield). MS (DCI/NH₃) m/e 163 (M+H)⁺.

### EXAMPLE 36D

### N-(3-methylphenyl)-2-[4-(2-pyridinyl)-1-piperidinyl]acetamide

The product from Example 36C (200 mg, 1 mmol), the product from Example 1A (228 mg, 1 mmol) and N,N-diisopropylethylamine (0.185 mL, 1.1 mmol) in toluene (8 mL) were stirred at 60° C. for 18 hours. The reaction mixture was poured into water (30 mL) and extracted with ethyl acetate (20 mL). The organic layer was washed with brine (2×30 mL), dried over MgSO₄, filtered, and the filtrate was concentrated under reduced pressure. The residue was purified by flash chromatography on silica gel (elution with ethyl acetate:ethanol, 9.2:0.8) to provide the title compound (169 mg, 55%). ¹H NMR (300 MHz, DMSO-d₆) δ 1.83 (m, 4H), 2.24 (m, 5H), 2.64 (m, 1H), 2.98 (m, 2H), 3.12 (s, 2H), 6.88 (d, 1H, J=6 Hz), 7.20 (m, 2H), 7.30 (d, 1H, J=6 Hz), 7.45 (d, 2H, J=6 Hz), 7.71 (m, 1H), 8.51 (m, 1H), 9.59 (br s, 1H); MS (DCI/NH₃) m/e 310 (M+H)⁺. Anal. calcd for C₁₉H₂₃N₃O-0.15H₂O: C, 73.12; H, 7.52; N, 13.46. Found: C, 72.72, H, 7.24, N, 13.28.

### EXAMPLE 37

### 2-[4-(2-fluorophenyl)-1-piperidinyl]1-N-(3-methylphenyl)acetamide

The procedure described in Example 35 was followed, substituting 4-(2-fluorophenyl)piperidine for 4-(2-methoxyphenyl)piperidine (89 mg, 80.9% yield), to provide the title compound (89 mg, 80.9% yield). ¹H NMR (300 MHz, DMSO-d₆) δ 1.72 (m, 2H), 1.85 (m, 2H), 2.29 (m, 5H), 2.51 (m, 1H), 2.80 (m, 1H), 2.97 (m, 2H), 3.12 (s, 2H), 6.88 (d, 1H, J=6 Hz), 7.19 (m, 4H), 7.42 (m, 3H), 9.61 (br s, 1H); MS (DCI/NH₃) m/e 327 (M+H)⁺. Anal. calcd for C₂₀H₂₃FN₂O: C, 73.59; H, 7.10; N, 8.58. Found: C, 73.49, H, 6.97, N, 8.30.

### EXAMPLE 38

### N-(3-methylphenyl)-2-[4-(2-methylphenyl)-1-piperidinyl]acetamide

The procedure described in Example 35 was followed, substituting 4-(2-methylphenyl)piperidine for 4-(2-methoxyphenyl)piperidine, to provide the title compound (65 mg, 87.8% yield). ¹H NMR (300 MHz, DMSO-d₆) δ 1.72 (m, 2H), 1.79 (m, 2H), 2.29 (m, 8H), 2.69 (m, 1H), 2.97 (m, 2H), 3.12 (s, 2H), 6.88 (d, 1H, J=6 Hz), 7.13 (m, 4H), 7.28 (d, 1H, J=6 Hz), 7.47 (m, 2H), 9.61 (br s, 1H); MS (DCI/NH₃) m/e 323 (M+H)⁺. Anal. calcd for C₂₁H₂₆N₂O: C, 78.22; H, 8.13; N, 8.69. Found: C, 77.86, H, 8.12, N, 8.51.

### EXAMPLE 39

### 2-[4-(3-fluorophenyl-1-piperidinyl]-N-(3-methylphenyl)acetamide

The procedure described in Example 35 was followed, substituting 4-(3-fluorophenyl)piperidine for 4-(2-methoxyphenyl)piperidine to provide the title compound (68 mg, 61.8% yield). ¹H NMR (300 MHz, DMSO-d₆) δ 1.75 (m, 4H), 2.29 (m, 5H), 2.55 (m, 1H), 2.96 (m, 2H), 3.12 (s, 2H), 6.88 (d, 1H, J=6 Hz), 7.01 (m, 1H), 7.14 (m, 3H), 7.35 (m, 1H), 7.45 (m, 2H), 9.61 (br s, 1H); MS (DCI/NH₃) m/e 327 (M+H)⁺.

### EXAMPLE 40

### N-(3-methylphenyl)-2-[4-(6-oxo-1 (6H)-pyridazinyl)-1-piperidinyl]acetamide

### EXAMPLE 40A

### tert-butyl 4-(6-oxo-1 (6H)-pyridazinyl)-1-piperidinecarboxylate

tert-Butyl 4-bromo-1-piperidinecarboxylate (1.00 g, 3.78 mmol) in DMF (20 mL) was treated with K₂CO₃ (523 mg, 3.78 mmol) and 3(2H)-pyridazinone (340 mg, 3.78 mmol) and then heated at 45° C. for 60 hours. The reaction mixture was allowed to cool to room temperature, poured into water (80 mL) and extracted with ethyl acetate (80 mL). The organic layer was washed with brine (3×50 mL), dried over MgSO₄, filtered, and the filtrate concentrated under reduced pressure. The residue was purified by flash chromatography on silica gel (elution with hexanes:ethyl acetate, 3:1) to provide the title compound (180 mg, 17% yield). ¹H NMR (300 MHz, DMSO-d₆) δ 1.41 (s, 9H), 1.66 (m, 4H), 2.91 (m, 2H), 4.05 (m, 2H), 4.96 (m, 1H), 6.93 (dd, 1H, J=1.5, 9.0 Hz), 7.39 (dd, 1H, J=3.0, 9.0 Hz), 7.95 (dd, 1H, J=3.0, 9.0 Hz); (MS (DCI/NH₃) m/e 280 (M+H)⁺.

### EXAMPLE 40B

### 2-(4-piperidinyl)-3 (2H)-pyridazinone

The product from Example 40A (180 mg, 0.6 mmol) in dichloromethane (5 mL) was cooled to 0° C. and treated with trifluoroacetic acid (TFA) (0.46 mL, 6 mmol). After stirring at 0° C. for 3 hours, the reaction mixture was allowed to warm to room temperature and stirred an additional 3 hours. The reaction mixture was concentrated under reduced pressure and the residue was azeotroped with toluene (2×30 mL) to provide the title compound as the TFA salt (180 mg). ¹H NMR (300 MHz, DMSO-d₆) δ 2.05 (m, 4H), 3.14 (m, 2H), 3.4 (m, 2H), 5.08 (m, 1H), 6.97 (dd, 1H, J=1.5, 9.0 Hz), 7.43 (dd, 1H, J=3.0, 9.0 Hz), 8.0 (dd, 1H, J=3.0, 9.0 Hz), 8.36 (br s, 1H), 8.70 (br s, 1H); (MS (DCI/NH₃) m/e 180 (M+H)⁺.

### EXAMPLE 40C

### N-(3-methylphenyl)-2-[4-(6-oxo-1 (6H)-pyridazinyl)-1-piperidinyl]acetamide

The product from Example 40B, as the TFA salt, (80 mg, 0.27 mmol), the product from Example 1A (0.062 g, 0.27 mmol), and K₂CO₃ (0.113 g, 0.81 mmol) were combined in toluene (8 mL) and stirred at room temperature for 18 hours. The reaction mixture was poured into water (30 mL) and extracted with ethyl acetate (20 mL). The organic layer was washed with brine (2×30 mL), dried over MgSO₄, filtered, and the filtrate concentrated under reduced pressure. The residue was purified by flash chromatography using ethyl acetate:ethanol, 9.7:0.3 to provide the title compound (89 mg, (100% yield). ¹H NMR (300 MHz, DMSO-d₆) δ 1.74 (m, 2H), 2.01 (m, 2H), 2.29 (s, 3H), 2.35 (m, 2H), 3.0 (m, 2H), 3.15 (s, 2H), 4.78 (m, 1H), 6.88 (d, 1H, J=7.5 Hz), 6.93 (dd, 1H, J=1.5, 9.0 Hz), 7.19 (t, 1H J=7.5 Hz), 7.38 (dd, 1H, J=3.0, 9.0 Hz), 7.43 (d, 2H, J=7.5 Hz), 7.98 (dd, 1H, J=3.0, 9.0 Hz), 9.31 (br s, 1H); MS (DCI/NH₃) m/e 327 (M+H)⁺.

### EXAMPLE 41

### N-(2,6-dimethylphenyl)-2-[4-(2-thienyl)-1-piperidinyl]acetamide

4-(2-Thienyl)piperidine hydrochloride (22 mg, 0.11 mmol), 2-chloro-N-(2,6-dimethylphenyl)acetamide (24 mg, 0.12 mmol), and sodium carbonate (50 mg) in N,N-dimethylformamide:water (2:1, 2 mL) was shaken at ambient temperature for 18 hours. The resulting mixture was concentrated under reduced pressure. The residue was purified by preparative HPLC to provide the title compound as a trifluoroacetic acid salt (30 mg, 62% yield). ¹H NMR (300 MHz, DMSO-d₆) δ 2.00 (m, 2H), 2.19 (s, 6H), 2.20 (m, 2H), 3.15-3.30 (m, 3H), 3.60 (m, 2H), 4.22 (s, 2H), 6.97 (m, 1H), 7.00 (m, 1H), 7.16 (m, 3H), 7.40 (d, 1H, J=3 Hz), 9.85 (br s, 1H), 9.95 (s, 1H); MS (ESI APCI+) m/e 329 (M+H)⁺.

### EXAMPLE 42

### N-(2,5-dimethylphenyl)-2-[4-(2-thienyl)-1-piperidinyl]acetamide

The procedure described in Example 41 was followed, substituting 2-chloro-N-(2,5-dimethylphenyl)acetamide for 2-chloro-N-(2,6-dimethylphenyl)acetamide, to provide the title compound (35 mg, 72% yield). ¹H NMR (300 MHz, DMSO-d₆) δ 2.00 (m, 2H), 2.19 (s, 3H), 2.20 (m, 2H), 2.25 (s, 3H), 3.18-3.30 (m, 3H), 3.60 (m, 2H), 4.20 (s, 2H), 6.97 (m, 1H), 7.00 (m, 2H), 7.18 (d, 1H, J=6 Hz), 7.22 (s, 1H), 7.40 (d, 1H, J=3 Hz), 9.80 (br s, 1H), 9.90 (s, 1H); MS (ESI APCI+) m/e 329 (M+H)⁺.

### EXAMPLE 43

### N-(2-methylphenyl)-2-[4-(2-thienyl)-1-piperidinyl]acetamide

The procedure described in Example 41 was followed, substituting 2-chloro-N-(2-methylphenyl)acetamide for 2-chloro-N-(2,6-dimethylphenyl)acetamide, to provide the title compound (30 mg, 64% yield). ¹H NMR (300 MHz, DMSO-d₆) δ 2.00 (m, 2H), 2.20 (m, 2H), 2.22 (s, 3H), 3.18-3.30 (m, 3H), 3.60 (m, 2H), 4.20 (s, 2H), 6.97 (m, 1H), 7.00 (m, 1H), 7.18 (m, 1H), 7.22 (m, 1H), 7.28 (m, 1H), 7.40 (m, 2H), 9.82 (br s, 1H), 10.00 (s, 1H); MS (ESI APCI+) m/e 315 (M+H)⁺.

### EXAMPLE 44

### N-(3-chloro-4-fluorophenyl)-2-[4-(2-thienyl) 1-piperidinyl] acetamide

The procedure described in Example 41 was followed, substituting 2-chloro-N-(3-chloro-4-fluorophenyl)acetamide for 2-chloro-N-(2,6-dimethylphenyl) acetamide, to provide the title compound (29 mg, 57% yield). ¹H NMR (300 MHz, DMSO-d₆) δ 2.00 (m, 2H), 2.20 (m, 2H), 3.18-3.30 (m, 3H), 3.60 (m, 2H), 4.20 (s, 2H), 6.97 (m, 1H), 7.00 (m, 1H), 7.42 (m, 3H), 7.95 (d, 1H, J=3 Hz), 9.90 (br s, 1H), 10.75 (br s, 1H); MS (ESI APCI+) m/e 353 (M+H)⁺.

### EXAMPLE 45

### N-(4-bromophenyl)-2-[4-(2-pyridinyl)-1-piperidinyl]acetamide

The product from Example 36C (hydrochloride, 20 mg, 0.10 mmol), N-(4-bromophenyl)-2-chloroacetamide (27 mg, 0.11 mmol), and sodium carbonate (50 mg) in DMF:water (2:1, 2 mL) was shaken at ambient temperature for 18 hours. The resulting mixture was decanted and concentrated under reduced pressure. The residue was purified by preparative HPLC to provide the title compound as a trifluoroacetic acid salt (34 mg, 70.9% yield). ¹H NMR (500 MHz, DMSO-d₆) δ 2.10 (m, 4H), 3.02 (m, 1H), 3.26 (m, 2H), 3.62 (m, 2H), 4.21 (s, 2H), 7.28 (m, 2H), 7.56 (m, 4H), 7.82 (t, 1H, J=6 Hz), 8.26 (d, 1H, J=6 Hz), 9.90 (br s, 1H), 10.20 (br s, 1H); MS (ESI APCI+) m/e 373 (M−H)⁺.

### EXAMPLE 46

### N-(2,6-dimethylphenyl)-2-[4-(2-pyridinyl)-1-piperidinyl]acetamide

The procedure described in Example 45 was followed, substituting 2-chloro-N-(2,6-dimethylphenyl)acetamide for N-(4-bromophenyl)-2-chloroacetamide, to provide the title compound (31 mg, 70.3% yield). ¹H NMR (500 MHz, DMSO-d₆) δ 2.10 (m, 4H), 2.18 (s, 6H), 3.02 (m, 1H), 3.31 (m, 2H), 3.62 (m, 2H), 4.25 (s, 2H), 7.12 (m, 3H), 7.32 (t, 1H, J=4 Hz), 7.28 (d, 1H, J=6 Hz), 7.82 (t, 1H, J=6 Hz), 8.66 (d, 1H, J=4 Hz), 9.90 (br s, 1H), 9.98 (s, 1H); MS (ESI APCI+) m/e 324 (M+H)⁺.

### EXAMPLE 47

### N-(2-nitrophenyl)-2-[4-(2-pyridinyl)-1-piperidinyl]acetamide

The procedure described in Example 45 was followed, substituting 2-chloro-N-(2-nitrophenyl)acetamide for N-(4-bromophenyl)-2-chloroacetamide, to provide the title compound (43 mg, 90% yield). ¹H NMR (500 MHz, DMSO-d₆) δ 2.10 (m, 4H), 3.02 (m, 1H), 3.31 (m, 2H), 3.62 (m, 2H), 4.25 (s, 2H), 7.22 (m, 2H), 7.45 (t, 1H, J=4 Hz), 7.65 (m, 1H), 7.80 (m, 2H), 8.01 (d, 1H, J=6 Hz), 8.58 (d, 1H, J=4 Hz), 10.00 (br s, 1H), 11.02 (s, 1H); MS (ESI APCI+) m/e 341 (M+H)⁺.

### EXAMPLE 48

### N-(3-nitrophenyl)-2-[4-(2-pyridinyl)-1-piperidinyl]acetamide

The procedure described in Example 45 was followed, substituting 2-chloro-N-(3-nitrophenyl)acetamide for N-(4-bromophenyl)-2-chloroacetamide, to provide the title compound (25 mg, 55% yield). ¹H NMR (500 MHz, DMSO-d₆) δ 2.10 (m, 4H), 3.02 (m, 1H), 3.31 (m, 2H), 3.62 (m, 2H), 4.25 (s, 2H), 7.38 (m, 2H), 7.70 (t, 1H, J=6 Hz), 7.82 (t, 1H, J=4 Hz), 7.92 (d, 1H, J=6 Hz), 9.02 (d, 1H, J=4 Hz), 8.58 (d, 1H, J=4 Hz), 8.65 (s, 1H), 10.00 (br s, 1H), 11.12 (s, 1H); MS (ESI APCI+) m/e 341 (M+H)⁺.

### EXAMPLE 49

### N-(2,4-difluorophenyl-2-[4-(2-pyridinyl) 1-piperidinyl]acetamide

The procedure described in Example 45 was followed, substituting 2-chloro-N-(2,4-difluorophenyl)acetamide for N-(4-bromophenyl)-2-chloroacetamide, to provide the title compound (26 mg, 59% yield). ¹H NMR (500 MHz, DMSO-d₆) δ 2.08 (m, 4H), 3.02 (m, 1H), 3.31 (m, 2H), 3.62 (m, 2H), 4.20 (s, 2H), 7.15 (m, 1H), 7.28 (m, 3H), 7.82 (m, 2H), 8.58 (d, 1H, J=4 Hz), 9.92 (br s, 1H), 10.52 (s, 1H); MS (ESI APCI+) m/e 332 (M+H)⁺.

### EXAMPLE 50

### N-(2,5-dimethylphenyl)-2-[4-(2-pyridinyl)-1-piperidinyl]acetamide

The procedure described in Example 45 was followed, substituting 2-chloro-N-(2,5-dimethylphenyl)acetamide for N-(4-bromophenyl)-2-chloroacetamide, to provide the title compound (12.2 mg, 28% yield). ¹H NMR (500 MHz, DMSO-d₆) δ 2.10 (m, 4H), 2.18 (s, 3H), 2.28 (s, 3H), 3.05 (m, 1H), 3.31 (m, 2H), 3.65 (m, 2H), 4.25 (s, 2H), 6.98 (d, 1H, J=6 Hz), 7.15 (d, 1H, J=6 Hz), 7.22 (s, 1H), 7.38 (m, 2H), 7.82 (t, 1H, J=4 Hz), 8.58 (d, 1H, J=4 Hz), 9.90 (br s, 1H), 9.98 (s, 1H); MS (ESI APCI+) m/e 324 (M+H)⁺.

### EXAMPLE 51

### N-(2-methylphenyl)-2-[4-(2-pyridinyl)-1-piperidinyl]acetamide

The procedure described in Example 45 was followed, substituting 2-chloro-N-(2-methylphenyl)acetamide for N-(4-bromophenyl)-2-chloroacetamide, to provide the title compound (16 mg, 37% yield). ¹H NMR (500 MHz, DMSO-d₆) δ 2.10 (m, 4H), 2.12 (s, 3H), 3.05 (m, 1H), 3.28 (m, 2H), 3.65 (m, 2H), 4.22 (s, 2H), 7.10 (m, 3H), 7.42 (m, 3H), 7.85 (t, 1H, J=4 Hz), 8.58 (d, 1H, J=4 Hz), 9.90 (br s, 1H), 9.98 (s, 1H); MS (ESI APCI+) m/e 310 (M+H)⁺.

### EXAMPLE 52

### N-(4-methylphenyl)-2-[4-(2-pyridinyl)-1-piperidinyl]acetamide

The procedure described in Example 45 was followed, substituting 2-chloro-N-(4-methylphenyl) acetamide for N-(4-bromophenyl)-2-chloroacetamide, to provide the title compound (29 mg, 68% yield). ¹H NMR (500 MHz, DMSO-d₆) δ 2.10 (m, 4H), 2.32 (s, 3H), 3.05 (m, 1H), 3.28 (m, 2H), 3.65 (m, 2H), 4.22 (s, 2H), 7.18 (d, 2H, J=6 Hz), 7.38 (t, 1H, J=4 Hz), 7.42 (d, 1H, J=4 Hz), 7.50 (d, 2H, J=6 Hz), 7.85 (t, 1H, J=4 Hz), 8.58 (d, 1H, J=4 Hz), 9.90 (br s, 1H), 10.55 (s, 1H); MS (ESI APCI+) m/e 310 (M+H)⁺.

### EXAMPLE 53

### 2-[4-(2-pyridinyl]-1-piperidin 11-N-[3-(trifluoromethyl)phenyl]acetamide

The procedure described in Example 45 was followed, substituting 2-chloro-N-(3-trifluromethylphenyl)acetamide for N-(4-bromophenyl)-2-chloroacetamide, to provide the title compound (34 mg, 71% yield). ¹H NMR (500 MHz, DMSO-d₆) δ 2.05 (m, 4H), 2.95 (m, 1H), 3.28 (m, 2H), 3.65 (m, 2H), 4.12 (s, 2H), 7.22 (t, 1H, J=4 Hz), 7.35 (d, 1H, J=4 Hz), 7.42 (d, 1H, J=4 Hz), 7.60 (t, 1H, J=4 Hz), 7.75 (t, 1H, J=4 Hz), 7.85 (d, 1H, J=4 Hz), 8.12 (s, 1H), 8.52 (d, 1H, J=4 Hz), 9.95 (br s, 1H), 10.75 (br s, 1H); MS (ESI APCI+) m/e 364 (M+H)⁺.

### EXAMPLE 54

### ethyl 4-({[4-(2-pyridinyl)-1-piperidinyl]acetyl}amino)benzoate

The procedure described in Example 45 was followed, substituting ethyl 4-[(chloroacetyl)amino]benzoate for N-(4-bromophenyl)-2-chloroacetamide, to provide the title compound (30 mg, 62% yield). ¹H NMR (500 MHz, DMSO-d₆) δ 1.28 (t, 3H, J=4 Hz), 1.98 (m, 6H), 2.41 (m, 2H), 2.72 (m, 1H), 3.01 (m, 2H), 3.20 (s, 2H), 4.28 (dd, 2H, J=4, 4 Hz), 7.22 (t, 1H, J=3 Hz), 7.30 (d, 1H, J=4 Hz), 7.78 (m, 3H), 7.95 (d, 2H, J=3 Hz), 8.55 (s, 1H), 9.90 (br s, 1H), 10.55 (br s, 1H); MS (ESI APCI+) m/e 368 (M+H)⁺.

### EXAMPLE 55

### N-(3-chloro-4-methylphenyl)-2-[4-(2-pyridinyl)-1-piperidinyl]acetamide

The procedure described in Example 45 was followed, substituting 2-chloro-N-(3-chloro-4-methylphenyl)acetamide for N-(4-bromophenyl)-2-chloroacetamide, to provide the title compound (33 mg, 72% yield). ¹H NMR (500 MHz, DMSO-d₆) δ 2.02 (m, 5H), 2.28 (s, 3H), 2.91 (m, 1H), 3.28 (m, 3H), 4.02 (s, 2H), 7.22 (t, 1H, J=4 Hz), 7.35 (d, 1H, J=4 Hz), 7.45 (d, 1H, J=4 Hz), 7.75 (t, 2H, J=4 Hz), 7.80 (s, 1H), 8.52 (d, 1H, J=4 Hz), 9.95 (br s, 1H), 10.75 (br s, 1H); MS (ESI APCI+) m/e 344 (M+H)⁺.

### EXAMPLE 56

### N-(2-cyanophenyl)-2-[4-(2-pyridinyl) 1-piperidinyl]acetamide

The procedure described in Example 45 was followed, substituting 2-chloro-N-(2-cyanophenyl)acetamide for N-(4-bromophenyl)-2-chloroacetamide, to provide the title compound (27 mg, 63% yield). ¹H NMR (500 MHz, DMSO-d₆) δ 2.02 (m, 4H), 2.91 (m, 1H), 3.28 (m, 2H), 3.65 (m, 2H), 4.22 (s, 2H), 7.22-7.45 (m, 3H), 7.75-7.85 (m, 4H), 8.52 (m, 1H), 9.95 (br s, 1H), 10.85 (br s, 1H); MS (ESI APCI+) m/e 321 (M+H)⁺.

### EXAMPLE 57

### N-(3-chlorophenyl)-2-[4-(2-pyridinyl)-1-piperidinyl]acetamide

The procedure described in Example 45 was followed, substituting 2-chloro-N-(3-chlorophenyl)acetamide for N-(4-bromophenyl)-2-chloroacetamide, to provide the title compound (16 mg, 36% yield). ¹H NMR (500 MHz, DMSO-d₆) δ 2.05 (m, 4H), 2.98 (m, 3H), 3.65 (m, 2H), 4.02 (s, 2H), 7.22 (t, 1H, J=4 Hz), 7.35 (d, 1H, J=4 Hz), 7.42 (d, 1H, J=4 Hz), 7.60 (t, 1H, J=4 Hz), 7.75 (t, 1H, J=4 Hz), 7.85 (d, 1H, J=4 Hz), 8.12 (s, 1H), 8.52 (d, 1H, J=4 Hz), 9.95 (br s, 1H), 10.75 (br s, 1H); MS (ESI APCI+) m/e 330 (M+H)⁺.

### EXAMPLE 58

### 2-[4-(3-cyano-2-pyridinyl)-1-piperidin]-N-(3-methylphenyl)acetamide

### EXAMPLE 58A

### benzyl 4-{[(trifluoromethyl)sulfonyl]oxy}-3,6-dihydro-1(2H)-pyridinecarboxylate

The title compound was prepared according to the procedure described in J. Org. Chem. 1998, 63, 8320. Benzyl 4-oxo-1-piperidinecarboxylate (0.5 g, 2.1 mmol) and N-phenytrifluoromethanesulfonimide (1.15 g, 3.2 mmol) in tetrahydrofuran (10 mL) at −78° C. was treated with lithium hexamethyldisilazide (2.14 mL, 2.1 mmol). After 4 hours at −78° C., the mixture was quenched with water and extracted with a large excess of diethyl ether (3×). The ethereal layers were combined, dried over sodium sulfate, filtered, and the filtrate concentrated under reduced pressure. The residue was chromatographed on flash silica gel (20% ethyl acetate:hexanes) to provide the title compound (0.471 g, 60% yield). ¹H NMR (300 MHz, CDCl₃) δ 2.47 (m, 2H), 3.72 (m, 2H), 4.13 (m, 2H), 5.16 (s, 2H), 5.78 (br m, 1H), 7.36 (m, 5H); A MS (ESI) m/e 366 (M+H)⁺.

### EXAMPLE 58B

### benzyl 4-(4.4,5,5-tetramethyl-1,32-dioxaborolan-2-yl)-3,6-dihydro-1 (2H)-pyridinecarboxylate

The title compound was prepared according to the procedure described in Tetrahedron Lett. 2000, 41 3705. Bis(pinacolato)diborane (338 mg, 1.33 mmol), potassium acetate (356 mg, 3.63 mmol), [1,1′-bis(diphenylphosphino)ferrocene]dichloropalladium(II) (PdCl₂dppf; 30 mg, 0.04 mmol), and 1,1′-bis(diphenylphosphino)ferrocene (20 mg, 0.04 mmol) were combined and treated with the product from Example 58A (440 mg, 1.21 mmol) in degassed 1,4-dioxane (7 mL). The reaction mixture was heated at 80° C. for 16 hours, allowed to cool to 23° C., diluted with water, and extracted with dichloromethane (3×). The dichloromethane extracts were combined, dried over sodium sulfate, filtered, and the filtrate concentrated under reduced pressure. The residue was chromatographed on flash silica gel (20% ethyl acetate:hexanes) to provide the title compound (323 mg, 78% yield). ¹H NMR (300 MHz, CDCl₃) δ 1.25 (s, 12H), 2.24 (m, 2H), 3.52 (dd, 2H, J=5.7, 5.7 Hz), 4.03 (dd, 2H, J=3, 6 Hz), 5.14 (s, 2H), 6.46 (br m, 1H), 7.32 (m, 5H); MS (ESI) m/e 344 (M+H)⁺.

### EXAMPLE 58C

### benzyl 3-cyano-3′,6′-dihydro-2,4′-bipyridine-1′(2′H)-carboxylate 4,4,5,5-tetramethyl-1,3,2-dioxaborolan-2-ol complex

The product from Example 58B (200 mg, 0.58 mmol), potassium carbonate (241 mg, 1.75 mmol), PdCl₂dppf (29 mg, 0.035 mmol), and 2-chloro-3-cyanopyridine (85 mg, 0.61 mmol) were combined in degassed N,N-dimethylformamide (4 mL). The reaction mixture was heated at 80° C. for 16 hours, allowed to cool to 23° C., diluted with water, dichloromethane, and the layers separated. The aqueous phase was extracted with dichloromethane (2×). All the dichloromethane phases were combined, dried over sodium sulfate, filtered, and the filtrate concentrated under reduced pressure. The residue was chromatographed on flash silica gel (50% ethyl acetate:hexanes) to provide the title compound sufficiently pure to carry on in further reactions (323 mg, 78% yield). ¹H NMR (300 MHz, CDCl₃) δ 1.13 (s, 12H), 2.74 (br s, 2H), 3.75 (dd, 2H, J=6 Hz), 4.26 (m, 2H), 5.19 (s, 2H), 6.57 (br m, 1H), 7.32 (m, 6H), 7.98 (dd, 1H, J=1.8, 7.8 Hz), 8.76 (dd, 1H, J=1.8, 4.5 Hz); MS (ESI) m/e 320 (M+H)⁺.

### EXAMPLE 58D

### 2-(4-piperidinyl)nicotinonitrile

A steady stream of H₂ was bubbled through a stirred solution of the product from Example 58C (70 mg, 0.15 mmol), Pd/C (5 mg), and ethanol (2 mL) at 23° C. for 24 hours. The H₂ bubbling was stopped and N₂ was bubbled through for a few minutes. The reaction mixture was passed through Celite and the filtrate concentrated under reduced pressure to provide the title compound sufficiently pure to carry into further reactions (30 mg). MS (ESI) m/e 188 (M+H)⁺.

### EXAMPLE 58E

### 2-[4-(3-cyano-2-pyridinyl)-1-piperidinyl]-N-(3-methylphenyl)acetamide

The product from Example 58D, the product from Example 1A (37 mg, 0.16 mmol), N, N-diisopropylethylamine (31 mg, 0.24 mmol), and toluene (3 mL) were combined and heated at 60° C. After 16 hours, the mixture was allowed to cool to 23° C. and concentrated under reduced pressure. The residue was purified by thin layer chromatography (7% ethyl acetate:hexanes) to provide the title compound (9 mg, 17% yield). ¹H NMR (400 MHz, DMSO-d₆) δ 1.79 (br d, 2H, J=12 Hz), 2.02 (m, 2H), 2.27 (s, 3H), 2.32 (m, 2H), 3.04 (m, 3H), 3.16 (s, 2H), 6.88 (bd, 1H, J=8 Hz), 7.18 (dd, 1H, J=7.2, 7.2 Hz), 7.45 (m, 3H), 8.26 (dd, 1H, J=1, 2 Hz), 8.82 (dd, 1H, J=1, 4.4 Hz), 9.58 (s, 1H); MS (APCI/ESI) m/e 335 (M+H)⁺.

### EXAMPLE 59

### N-(3-methylphenyl)-2-(4-phenyl-3,6-dihydro-1(2H)-pyridinyl)acetamide

The procedure described in Example 35 was followed, substituting 4-phenyl-1,2,3,6-tetrahydropyridine hydrochloride for 4-(2-methoxyphenyl)piperidine, to provide the title compound (180 mg, 39% yield). ¹H NMR (300 MHz, DMSO-d₆) δ 2.27 (s, 3H), 2.55 (m, 2H), 2.78 (t, 2H, J=6 Hz), 3.26 (m, 4H), 6.18 (m, 1H), 6.88 (m, 1H). 7.17 (t, 1H, J=7.5 Hz), 7.25 (m, 1H), 7.35 (m, 2H), 7.45 (m, 4H), 9.64 (s, 1H); MS (DCI/NH₃) m/e 307 (M+H)⁺. Anal. calcd for C₂₀H₂₂N₂O_{0.10}H₂O: C, 77.94; H, 7.26; N, 9.09. Found: C, 77.72, H, 7.28, N, 9.03.

### EXAMPLE 60

### 2-(3′ 6′-dihydro-24′-b din-1′(2′H)-yl)-N-(3-methylphenyl)acetamide

The procedure described in Example 35 was followed, substituting 1′,2′,3′,6′-tetrahydro-2,4′-bipyridine hydrochloride (Saari, W. S.; et al. J. Med. Chem. 1984, 27, 1182) for 4-(2-methoxyphenyl)piperidine, to provide the title compound (210 mg, 53.8% yield). ¹H NMR (300 MHz, DMSO-d₆) δ 2.27 (s, 3H), 2.65 (m, 2H), 2.78 (t, 2H, J=6 Hz), 3.25 (s, 2H), 3.30 (m, 2H), 6.71 (m, 1H), 6.88 (m, 1H). 7.18 (t, 1H, J=7.5 Hz), 7.23 (m, 1H), 7.45 (m, 2H), 7.55 (d, 1H, J=9 Hz), 7.75 (m, 1H), 8.53 (m, 1H), 9.64 (br s, 1H); MS (DCI/NH₃) m/e 308 (M+H)⁺. Anal. calcd for C₁₉H₂₁N₃O.0.30H₂O: C, 72.96; H, 6.96; N, 13.43. Found: 72.73, H, 6.57, N, 13.47.

### EXAMPLE 61

### 2-(3′,6′-dihydro-2,4′-bipyridin-1′(2′H)-yl)-N-(2,6-dimethylphenyl)acetamide

1′,2′,3′,6′-Tetrahydro-2,4′-bipyridine hydrochloride (22 mg, 0.11 mmol), 2-chloro-N-(2,6-dimethylphenyl)acetamide (24 mg, 0.12 mmol), and sodium carbonate (50 mg) in DMF:water (2:1, 2 mL) were combined and shaken at ambient temperature for 18 hours. The mixture was concentrated under reduced pressure. The residue was purified by preparative HPLC to provide the title compound as a trifluoroacetic acid salt (43 mg, 90% yield). ¹H NMR (300 MHz, DMSO-d₆) δ 2.20 (s, 6H), 2.96 (br s, 2H), 3.43-3.63 (m, 2H), 4.03-4.20 (m, 2H), 4.39 (s, 2H), 6.72 (br s, 1H), 7.12 (m, 3H), 7.38 (m, 1H), 7.62 (d, 1H, J=6 Hz), 7.82 (m, 1H), 8.59 (m, 1H), 10.00 (m, 1H), 10.40 (br s, 1H); MS (ESI APCI+) m/e 322 (M+H)⁺.

### EXAMPLE 62

### 2-(3′,6′-dihydro-2,4′-bi 1′(2′H)-yl-N-(2-nitrophenyl)acetamide

The procedure described in Example 61 was followed, substituting 2-chloro-N-(2-nitrophenyl)acetamide for 2-chloro-N-(2,6-dimethylphenyl)acetamide, to provide the title compound (27 mg, 54% yield). ¹H NMR (300 MHz, DMSO-d₆) δ 2.93 (br s, 2H), 3.40-3.70 (m, 2H), 4.00-4.20 (m, 2H), 4.38 (s, 2H), 6.75 (br s, 1H), 7.38 (m, 1H), 7.48 (t, 1H, J=6 Hz), 7.62 (m, 2H), 7.80 (t, 1H, J=6 Hz), 7.88 (t, 1H, J=6 Hz), 8.03 (m, 1H), 8.60 (br s, 1H), 10.44 (br s, 1H), 10.98 (br s, 1H); MS (ESI APCI+) m/e 339 (M+H)⁺.

### EXAMPLE 63

### 2-(3′,6′-dihydro-2,4′-bipyridin-1′(2′H)-yl)-N-(3-nitrophenyl)acetamide

The procedure described in Example 61 was followed, substituting 2-chloro-N-(3-nitrophenyl)acetamide for 2-chloro-N-(2,6-dimethylphenyl)acetamide, to provide the title compound (48 mg, 97% yield). ¹H NMR (300 MHz, DMSO-d₆) δ 2.96 (br s, 2H), 3.45-3.75 (m, 2H), 4.05-4.20 (m, 2H), 4.38 (s, 2H), 6.75 (br s, 1H), 7.38 (m, 1H), 7.66 (m, 2H), 7.89 (m, 2H), 8.00 (d, 1H, J=6 Hz), 8.60 (m, 1H), 8.63 (br s, 1H), 10.45 (br s, 1H), 11.08 (br s, 1H); MS (ESI APCI+) m/e 339 (M+H)⁺.

### EXAMPLE 64

### 2-(3′,6′-dihdro-24′-bipyridin-1′(2′H)-yl)-N-(4-fluorophenyl)acetamide

The procedure described in Example 61 was followed, substituting 2-chloro-N-(4-fluorophenyl)acetamide for 2-chloro-N-(2,6-dimethylphenyl)acetamide, to provide the title compound (40 mg, 86% yield). ¹H NMR (300 MHz, DMSO-d₆) δ 2.96 (br s, 2H), 3.45-3.75 (m, 2H), 4.00-4.20 (m, 2H), 4.35 (s, 2H), 6.72 (br s, 1H), 7.22 (t, 2H, J=7 Hz), 7.38 (m, 1H), 7.63 (m, 3H), 7.82 (m, 1H), 8.60 (m, 1H), 10.38 (br s, 1H), 10.62 (br s, 1H); MS (ESI APCI+) m/e 312 (M+H)⁺.

### EXAMPLE 65

### N-(2,4-difluorophenyl)-2-(3′,6′-dihydro-2,4′-bipyridin-1′(2′H)-yl)acetamide

The procedure described in Example 61 was followed, substituting 2-chloro-N-(2,4-difluorophenyl)acetamide for 2-chloro-N-(2,6-dimethylphenyl)acetamide, to provide the title compound (45 mg, 92% yield). ¹H NMR (300 MHz, DMSO-d₆) δ 2.95 (br s, 2H), 3.45-3.70 (m, 2H), 4.00-4.20 (m, 2H), 4.35 (s, 2H), 6.74 (br s, 1H), 7.18 (t, 1H, J=7 Hz), 7.36 (m, 2H), 7.63 (m, 1H), 7.85 (m, 2H), 8.60 (m, 1H), 10.40 (br s, 1H), 10.45 (br s, 1H); MS (ESI APCI+) m/e 330 (M+H)⁺.

### EXAMPLE 66

### 2-(3′,6′-dihydro-2,4′-bipyridin-1′(2′H)-yl)-N-(2,5-dimethylphenyl)acetamide

The procedure described in Example 61 was followed, substituting 2-chloro-N-(2,5-dimethylphenyl)acetamide for 2-chloro-N-(2,6-dimethylphenyl)acetamide, to provide the title compound (28 mg, 59% yield). ¹H NMR (300 MHz, DMSO-d₆) δ 2.20 (s, 3H), 2.25 (s, 3H), 2.95 (br s, 2H), 3.45-3.70 (m, 2H), 4.00-4.20 (m, 2H), 4.35 (s, 2H), 6.74 (br s, 1H), 7.00 (d, 1H, J=7 Hz), 7.18 (d, 1H, J=7 Hz), 7.22 (s, 1H), 7.38 (m, 1H), 7.63 (d, 1H, J=7 Hz), 7.82 (m, 1H), 8.60 (m, 1H), 9.92 (br s, 1H), 10.35 (br s, 1H); MS (ESI APCI+) m/e 322 (M+H)⁺.

### EXAMPLE 67

### 2-(3′,6′-dihydro-2,4′-bipyridin-1′(2′H)-yl)-N-(2-methylphenyl)acetamide

The procedure described in Example 61 was followed, substituting 2-chloro-N-(2-methylphenyl)acetamide for 2-chloro-N-(2,6-dimethylphenyl)acetamide, to provide the title compound (30 mg, 65% yield). ¹H NMR (300 MHz, DMSO-d₆) δ 2.25 (s, 3H), 2.95 (br s, 2H), 3.45-3.70 (m, 2H), 4.00-4.20 (m, 2H), 4.35 (s, 2H), 6.75 (br s, 1H), 7.18 (m,1H), 7.22 (m, 1H), 7.28 (m, 1H), 7.38 (m, 1H), 7.43 (d, 1H, J=7 Hz), 7.63 (d, 1H, J=7 Hz), 7.82 (m, 1H), 8.60 (m, 1H), 9.96 (br s, 1H), 10.35 (br s, 1H); MS (ESI APCI+) m/e 308 (M+H)⁺.

### EXAMPLE 68

### N-cyclohexyl-2-(3′,6′-dihydro-2,4′-bipyridin-1′(2′H)-yl)acetamide

The procedure described in Example 61 was followed, substituting 2-chloro-N-cyclohexylacetamide for 2-chloro-N-(2,6-dimethylphenyl)acetamide, to provide the title compound (20 mg, 44% yield). ¹H NMR (300 MHz, DMSO-d₆) δ 1.10-1.35 (m, 5H), 1.60-1.80 (m, 5H), 2.95 (br s, 2H), 3.45-3.70 (m, 3H), 4.00-4.20 (m, 2H), 4.35 (s, 2H), 6.68 (br s, 1H), 7.38 (m, 1H), 7.63 (d, 1H, J=7 Hz), 7.82 (m, 1H), 8.42 (d, 1H, J=7 Hz), 8.60 (m, 1H), 10.25 (br s, 1H); MS (ESI APCI+) m/e 300 (M+H)⁺.

### EXAMPLE 70

### 2-(3 ′.6′-dihydro-2.4°-bipyridin-1′(2′H)-yl)-N-(4-methylphenyl)acetamide

The procedure described in Example 61 was followed, substituting 2-chloro-N-(4-methylphenyl)acetamide for 2-chloro-N-(2,6-dimethylphenyl)acetamide, to provide the title compound (26 mg, 56% yield). ¹H NMR (300 MHz, DMSO-d₆) δ 2.24 (s, 3H), 2.95 (br s, 2H), 3.45-3.70 (m, 2H), 4.00-4.20 (m, 2H), 4.35 (s, 2H), 6.74 (br s, 1H), 7.19 (d, 2H, J=7 Hz), 7.38 (m, 1H), 7.49 (d, 2H, J=7 Hz), 7.63 (d, 1H, J=7 Hz), 7.82 (m, 1H), 8.60 (m, 1H), 10.35 (br s, 1H), 10.45 (br s, 1H); MS (ESI APCI+) m/e 308 (M+H)⁺.

### EXAMPLE 71

### 2-(3′,6′-dihydro-2,4′-bipyridin-1′(2′H)-yl)-N-[3-(trifluoromethyl)phenyl]acetamide

The procedure described in Example 61 was followed, substituting 2-chloro-N-[3-(trifluoromethyl)phenyl]acetamide for 2-chloro-N-(2,6-dimethylphenyl) acetamide, to provide the title compound (47 mg, 90% yield). ¹H NMR (300 MHz, DMSO-d₆) δ 2.95 (br s, 2H), 3.45-3.70 (m, 2H), 4.00-4.20 (m, 2H), 4.35 (s, 2H), 6.74 (br s, 1H), 7.38 (m, 1H), 7.49 (d, 1H, J=6 Hz), 7.63 (m, 2H), 7.79 (d, 1H, J=6 Hz), 7.82 (m, 1H), 8.08 (s, 1H), 8.60 (m, 1H), 10.45 (br s, 1H), 10.98 (br s, 1H); MS (ESI APCI+) m/e 362 (M+H)⁺.

### EXAMPLE 72

### ethyl 4-[(3′,6′-dihydro-2,4′-bipyridin-1′(2′H)-ylacetyl)amino]benzoate

The procedure described in Example 61 was followed, substituting ethyl 4-[(chloroacetyl)amino]benzoate for 2-chloro-N-(2,6-dimethylphenyl)acetamide, to provide the title compound (51 mg, 97% yield). ¹H NMR (300 MHz, DMSO-d₆) δ 1.35 (t, 3H, J=7 Hz), 2.95 (br s, 2H), 3.45-3.70 (m, 2H), 4.00-4.20 (m, 2H), 4.22 (q, 2H, J=7 Hz), 4.36 (s, 2H), 6.74 (br s, 1H), 7.38 (m, 1H), 7.62 (d, 1H, J=6 Hz), 7.74 (d, 2H, J=8 Hz), 7.82 (m, 1H), 7.98 (d, 2H, J=8 Hz), 8.60 (m, 1H), 10.40 (br s, 1H), 10.92 (br s, 1H); MS (ESI APCI+) m/e 366 (M+H)⁺.

### EXAMPLE 73

### N-[2-chloro-5-(trifluoromethyl)phenyl]-2-(3′,6′-dihydro-2,4′-bipyridin-1′(2′H)-yl)acetamide

The procedure described in Example 61 was followed, substituting 2-chloro-N-[2-chloro-5-(trifluoromethyl)phenyl]acetamide for 2-chloro-N-(2,6-dimethylphenyl)acetamide, to provide the title compound (18 mg, 32% yield). ¹H NMR (300 MHz, DMSO-d₆) δ 2.95 (br s, 2H), 3.45-3.70 (m, 2H), 4.00-4.20 (m, 2H), 4.35 (s, 2H), 6.73 (br s, 1H), 7.38 (m, 1H), 7.63 (m, 2H), 7.82 (m, 2H), 8.22 (s, 1H), 8.60 (m, 1H), 10.40 (br s, 1H), 10.50 (br s, 1H); MS (ESI APCI+) m/e 396 (M+H)⁺.

### EXAMPLE 74

### N-(3-chloro-4-methylphenyl-2-(3′,6′-dihydro-2,4′-bipyridin-1′(2′H)-yl)acetamide

The procedure described in Example 61 was followed, substituting 2-chloro-N-(3-chloro-4-methylphenyl)acetamide for 2-chloro-N-(2,6-dimethylphenyl)acetamide, to provide the title compound (44 mg, 88% yield). ¹H NMR (300 MHz, DMSO-d₆) δ 2.28 (s, 3H), 2.95 (br s, 2H), 3.45-3.75 (m, 2H), 4.00-4.20 (m, 2H), 4.30 (s, 2H), 6.73 (br s, 1H), 7.38 (m, 3H), 7.63 (d, 1H, J=6 Hz), 7.80 (s, 1H), 7.83 (m, 1H), 8.60 (m, 1H), 10.38 (br s, 1H), 10.63 (br s, 1H); MS (ESI APCI+) m/e 342 (M+H)⁺.

### EXAMPLE 75

### N-(2-cyanophenyl)-2-(3′,6′-dihydro-2,4′-bipyridin-1′(2′H)-yl)acetamide

The procedure described in Example 61 was followed, substituting 2-chloro-N-(2-cyanophenyl)acetamide for 2-chloro-N-(2,6-dimethylphenyl)acetamide, to provide the title compound (46 mg, 97% yield). ¹H NMR (300 MHz, DMSO-d₆) δ 2.95 (br s, 2H), 3.45-3.75 (m, 2H), 4.00-4.20 (m, 2H), 4.40 (s, 2H), 6.75 (br s, 1H), 7.38 (m, 1H), 7.42 (t, 1H, J=6 Hz), 7.63 (m, 2H), 7.78 (m, 1H), 7.83 (m, 1H), 7.88 (m, 1H), 8.60 (m, 1H), 10.42 (br s, 1H), 10.93 (br s, 1H); MS (ESI APCI+) m/e 319 (M+H)⁺.

### EXAMPLE 76

### N-(3-chlorophenyl)-2-(3′,6′-dihdro-2,4′-b din-1′(2′H)-yl)acetamide

The procedure described in Example 61 was followed, substituting 2-chloro-N-(3-chlorophenyl)acetamide for 2-chloro-N-(2,6-dimethylphenyl)acetamide, to provide the title compound (42 mg, 86% yield). ¹H NMR (300 MHz, DMSO-d₆) δ 2.95 (br s, 2H), 3.45-3.75 (m, 2H), 4.00-4.20 (m, 2H), 4.34 (s, 2H), 6.75 (br s, 1H), 7.20 (d, 1H, J=6 Hz), 7.38 (m, 1H), 7.42 (m, 2H), 7.63 (d, 1H, J=6 Hz), 7.80 (s, 1H), 7.83 (m, 1H), 8.60 (m, 1H), 10.40 (br s, 1H), 10.80 (br s, 1H); MS (ESI APCI+) m/e 328 (M+H)⁺.

### EXAMPLE 77

### 1 N-(3-chloro-4-fluorophenyl)-2-(3′,6′-dihdro-2,4′-bipyridin-1′[(2H-yl)acetamide

The procedure described in Example 61 was followed, substituting 2-chloro-N-(3-chloro-4-fluorophenyl)acetamide for 2-chloro-N-(2,6-dimethylphenyl)acetamide, to provide the title compound (43 mg, 85% yield). ¹H NMR (300 MHz, DMSO-d₆) δ 2.95 (br s, 2H), 3.45-3.75 (m, 2H), 4.00-4.20 (m, 2H), 4.38 (s, 2H), 6.73 (br s, 1H), 7.38 (m, 11H), 7.43 (m, 2H), 7.63 (d, 11H, J=6 Hz), 7.83 (m, 1H), 7.92 (d, 1H, J=5 Hz), 8.60 (m, 1H), 10.38 (br s, 1H), 10.83 (br s, 1H); MS (ESI APCI+) m/e 346 (M+H)⁺.

### EXAMPLE 78

### 2-(3′,6′-dihydro-2.4°-bipyridin-1′(2′H)-yl)-N-[4-(trifluoromethoxy)phenyl]acetamide

The procedure described in Example 61 was followed, substituting 2-chloro-N-[4-(trifluoromethoxy)phenyl]acetamide for 2-chloro-N-(2,6-dimethylphenyl) acetamide, to provide the title compound (44 mg, 81% yield). ¹H NMR (300 MHz, DMSO-d₆) δ 2.95 (br s, 2H), 3.45-3.75 (m, 2H), 4.00-4.20 (m, 2H), 4.35 (s, 2H), 6.73 (br s, 1H), 7.38 (m, 1H), 7.41 (d, 2H, J=7 Hz), 7.63 (d, 1H, J=6 Hz), 7.73 (d, 2H, J=7 Hz), 7.84 (m, 1H), 8.60 (m, 1H), 10.40 (br s, 1H), 10.80 (br s, 1H); MS (ESI APCI+) m/e 378 (M+H)⁺.

### EXAMPLE 79

### 2-(3′,6′-dihydro-2,4′-bipyridin-1′(2′H)-yl)-N-[2-(trifluoromethyl)phenyl]acetamide

The procedure described in Example 61 was followed, substituting 2-chloro-N-[2-(trifluoromethyl)phenyl]acetamide 2-chloro-N-(2,6-dimethylphenyl)acetamide, to provide the title compound (41 mg, 78% yield). ¹H NMR (300 MHz, DMSO-d₆) δ 2.95 (br s, 2H), 3.45-3.75 (m, 2H), 4.00-4.20 (m, 2H), 4.35 (s, 2H), 6.73 (br s, 1H), 7.38 (m, 1H), 7.50-7.70 (m, 3H), 7.80-7.90 (m, 3H), 8.60 (m, 1H), 10.40 (s, 1H), 10.43 (br s, 1H); MS (ESI APCI+) m/e 362 (M+H)⁺.

### EXAMPLE 80

### N-(4-chlorophenyl)-2-(3′,61-dihydro-24′-bi din-1′(2′H)-yl)acetamide

The procedure described in Example 61 was followed, substituting 2-chloro-N-(4-chlorophenyl)acetamide for 2-chloro-N-(2,6-dimethylphenyl)acetamide, to provide the title compound (39 mg, 80% yield). ¹H NMR (300 MHz, DMSO-d₆) δ 2.95 (br s, 2H), 3.45-3.75 (m, 2H), 4.00-4.20 (m, 2H), 4.30 (s, 2H), 6.73 (br s, 1H), 7.38 (m, 1H), 7.44 (d, 2H, J=7 Hz), 7.63 (m, 3H), 7.83 (m, 1H), 8.60 (m, 1H), 10.40 (br s, 1H), 10.63 (s, 1H); MS (ESI APCI+) m/e 328 (M+H)⁺.

### EXAMPLE 81

### N-(2.3-dichlorophenyl)-2-(3′,6′-dihydro-2,4′-bipyridin-1′(2′H)-yl)acetamide

The procedure described in Example 61 was followed, substituting 2-chloro-N-(2,3-dichlorophenyl)acetamide for 2-chloro-N-(2,6-dimethylphenyl)acetamide, to provide the title compound (37 mg, 70% yield). ¹H NMR (300 MHz, DMSO-d₆) δ 2.95 (br s, 2H), 3.45-3.75 (m, 2H), 4.00-4.20 (m, 2H), 4.40 (s, 2H), 6.73 (br s, 1H), 7.38 (m, 1H), 7.42 (t, 11H, J=7 Hz), 7.58 (d, 1H, J=7 Hz), 7.64 (d, 1H, J=7 Hz), 7.72 (d, 1H, J=7 Hz), 7.83 (m, 1H), 8.60 (m, 1H), 10.40 (s, 1H), 10.43 (br s, 1H); MS (ESI APCI+) m/e 363 (M+H)⁺.

### EXAMPLE 82

### N-(3,5-dichlorophenyl)-2-(3′,6′-dihydro-2,4′-bipyridin-1′(2′H)-yl)acetamide

The procedure described in Example 61 was followed, substituting 2-chloro-N-(3,5-dichlorophenyl)acetamide for 2-chloro-N-(2,6-dimethylphenyl)acetamide, to provide the title compound (31 mg, 59% yield). ¹H NMR (300 MHz, DMSO-d₆) δ 2.95 (br s, 2H), 3.45-3.75 (m, 2H), 4.00-4.20 (m, 2H), 4.34 (s, 2H), 6.73 (br s, 1H), 7.38 (m, 1H), 7.40 (s, 1H), 7.63 (m, 3H), 7.83 (m, 1H), 8.60 (m, 1H), 10.40 (br s, 1H), 10.90 (s, 1H); MS (ESI APCI+) m/e 363 (M+H)⁺.

### EXAMPLE 83

### 2-(3′,6′-dihydro-2,4′-bipyridin-1′(2′H)-yl)-N-(4-fluoro-2-methylphenyl)acetamide

The procedure described in Example 61 was followed, substituting 2-chloro-N-(4-fluoro-2-methylphenyl)acetamide for 2-chloro-N-(2,6-dimethylphenyl)acetamide, to provide the title compound (34 mg, 70% yield). ¹H NMR (300 MHz, DMSO-d₆) δ 2.20 (s, 3H), 2.96 (br s, 2H), 3.43-3.63 (m, 2H), 4.03-4.20 (m, 2H), 4.39 (s, 2H), 6.72 (br s, 1H), 7.10 (m, 2H), 7.38 (m, 1H), 7.43 (m, 1H), 7.64 (d, 1H, J=7 Hz), 7.84 (m, 1H), 8.60 (m, 1H), 10.00 (m, 1H), 10.40 (br s, 1H); MS (ESI APCI+) m/e 326 (M+H)⁺.

### EXAMPLE 84

### N-(4-fluorophenyl)-2-[4-(2-pyridinyl)-1-piperidinyl]acetamide

The procedure described in Example 45 was followed, substituting 2-chloro-N-(4-fluorophenyl)acetamide for N-(4-bromophenyl)-2-chloroacetamide, to provide the title compound (57.5 mg, 59% yield). ¹H NMR (500 MHz, DMSO-d₆) δ 2.15 (m, 4H), 3.01 (m, 1H), 3.26 (m, 2H), 3.65 (m, 2H), 4.18 (s, 2H), 7.22 (m, 2H), 7.35 (m, 2H), 7.58 (m, 2H), 7.82 (m, 1H), 8.60 (m, 1H), 9.95 (br s, 1H), 10.65 (br s, 1H); MS (ESI APCI+) m/e 314 (M+H)⁺.

### EXAMPLE 85

### N-(3,5-dichlorophenyl)-2-[4-(2-pyridinyl)-1-piperidinyl]acetamide

The procedure described in Example 45 was followed, substituting 2-chloro-N-(4-fluorophenyl)acetamide for N-(4-bromophenyl)-2-chloroacetamide, to provide the title compound (18.5 mg, 39% yield). ¹H NMR (500 MHz, DMSO-d₆) δ 1.85 (m, 2H), 1.95 (m, 2H), 2.25 (m, 2H), 2.68 (m, 1H), 2.95 (m, 2H), 3.20 (s, 2H), 7.20 (m, 1H), 7.30 (m, 2H), 7.75 (m, 2H), 7.82 (s, 2H), 8.52 (s, 1H), 10.05 (br s, 1H); MS (ESI APCI+) m/e 365 (M+H)⁺.

### EXAMPLE 86

### N-(2,3-dichlorophenyl)-2-[4-(2-pyridinyl)-1-piperidinyl]acetamide

The procedure described in Example 45 was followed, substituting 2-chloro-N-(2,3-dichlorophenyl)acetamide for N-(4-bromophenyl)-2-chloroacetamide, to provide the title compound (18 mg, 38% yield). ¹HNMR (500 MHz, DMSO-d₆) δ 1.90 (m, 4H), 2.42 (m, 2H), 2.75 (m, 1H), 3.05 (m, 2H), 3.28 (s, 2H), 7.22 (t, 1H, J=3 Hz), 7.30 (d, 1H, J=3 Hz), 7.42 (m, 3H), 7.75 (t, 1H, J=3 Hz), 8.25 (s, 1H), 8.50 (s, 1H), 10.18 (br s, 1H); MS (ESI APCI+) m/e 365 (M+H)⁺.

### EXAMPLE 87

### 2-[4-(2-pyridinyl)-1-piperidinyl]-N-[2-(trifluoromethyl)phenyl]acetamide

The procedure described in Example 45 was followed, substituting 2-chloro-N-[2-(trifluoromethyl)phenyl]acetamide for N-(4-bromophenyl)-2-chloroacetamide, to provide the title compound (22 mg, 46% yield). ¹H NMR (500 MHz, DMSO-d₆) δ 1.85 (m, 4H), 2.41 (m, 2H), 2.72 (m, 1H), 3.01 (m, 2H), 3.20 (s, 2H), 7.22 (t, 1H, J=3 Hz), 7.30 (d, 1H, J=3 Hz), 7.38 (m,1H), 7.72 (m, 4H), 8.25 (br s, 1H), 8.50 (s, 1H), 9.98 (br s, 1H); MS (ESI APCI+) m/e 364 (M+H)⁺.

### EXAMPLE 88

### N-(3-chloro-4-fluorophenyl)-2-[4-(2-pyridinyl)-1-piperidinyl]acetamide

The procedure described in Example 45 was followed, substituting 2-chloro-N-(3-chloro-4-fluorophenyl)acetamide for N-(4-bromophenyl)-2-chloroacetamide, to provide the title compound (19 mg, 43% yield). ¹H NMR (500 MHz, DMSO-d₆) δ 1.95 (m, 4H), 2.41 (m, 2H), 2.72 (m, 1H), 3.01 (m, 2H), 3.20 (s, 2H), 7.25 (t, 1H, J=3 Hz), 7.30 (d, 1H, J=3 Hz), 7.40 (t, 1H, J=3 Hz), 7.55 (s, 1H), 7.75 (t, 1H, J=3 Hz), 7.95 (t, 1H, J=3 Hz), 8.45 (s, 1H), 9.90 (br s, 1H), 10.35 (br s, 1H); MS (ESI APCI+) m/e 348 (M+H)⁺.

### EXAMPLE 89

### 2-[4-(2-pyridinyl)-1-piperidinyl]-N-[4-(trifluoromethoxy)phenyl]acetamide

The procedure described in Example 45 was followed, substituting 2-chloro-N-[4-(trifluoromethoxy)phenyl]acetamide for N-(4-bromophenyl)-2-chloroacetamide, to provide the title compound (21 mg, 43% yield). ¹H NMR (500 MHz, DMSO-d₆) δ 1.91 (m, 4H), 2.41 (m, 2H), 2.65 (m, 1H), 3.05 (m, 2H), 3.20 (s, 2H), 7.12 (m, 1H), 7.25 (m, 3H), 7.85 (m, 4H), 8.50 (s, 1H), 9.90 (br s, 1H); MS (ESI APCI+) m/e 380 (M+H)⁺.

### EXAMPLE 90

### N-Cyclohexyl-2-(3′4′,5′,6′-tetrahydro-2′H-[2,4′]bipyridinyl-1′-yl) acetamide

The procedure described in Example 45 was followed, substituting 2-chloro-N-cyclohexylacetamide for N-(4-bromophenyl)-2-chloroacetamide, to provide the title compound (49 mg, 53% yield). ¹H NMR (500 MHz, DMSO-d₆) δ 1.10-1.35 (m, 6H), 1.55 (m, 1H), 1.70 (m, 2H), 1.79 (m, 2H), 2.08 (m, 4H), 2.98 (m, 1H), 3.18 (m, 2H), 3.68 (m, 2H), 3.88 (s, 2H), 7.38 (m, 2H), 7.82 (t, 1H, J=4 Hz), 8.45 (d, 1H, J=4 Hz), 8.58 (d, 1H, J=3 Hz), 9.70 (br s, 1H); MS (ESI APCI+) m/e 302 (M+H)⁺.

### EXAMPLE 91

### N-{[4-(2-cyanophenyl)-1-piperazinyl]methyl}-3-methylbenzamide

### EXAMPLE 91A

### [(3-methylbenzoyl)amino]methyl Acetate

N-(3-Methylbenzoyl)glycine (10 g, 51.7 mmol), lead tetraacetate (25.25 g, 56.94 mmol), and copper (II) acetate monohydrate (0.94 g, 5.17 mmol) were combined in toluene and heated at reflux overnight. The reaction mixture was to cool to room temperature, filtered through Celite and the fitlrate concentrated under reduced pressure. The residue was purified by flash column chromatography on silica gel (elution with 25% ethyl acetate/hexanes) to provide the title compound (7.95 g, 74% yield). ¹H NMR (300 MHz, CDCl₃) δ 2.10 (s, 3H), 2.40 (s, 3H), 5.45 (d, 2H, J=9 Hz), 7.35 (m, 2H), 7.55 (m, 1H), 7.62 (s, 1H); MS (DCI/NH₃) m/e 208 (M+H)⁺.

### EXAMPLE 91B

### N-[4-(2-Cyanophenyl)piperazin-1-ylmethyl]-3-methyl Benzamide

The product from Example 91A (4.00 g, 19.2 mmol), 1-(2-cyanophenyl)piperazine (3.6 g, 19.2 mmol), and triethylamine (5.3 ml, 38.4 mmol) were combined in acetonitrile (100 mL) and stirred overnight room temperature. The reaction mixture was concentrated under reduced pressure and the residue was purified by flash column chromatography on silica gel (elution with ethyl acetate) to provide the title compound as a colorless oil (2.85 g, 44% yield). ¹H NMR (300 MHz, CDCl₃) δ 2.40 (s, 3H), 2.90 (m, 4H), 3.25 (m, 4H), 4.45 (d, 2H, J=6 Hz), 6.66 (br s, 1H), 7.0 (m, 2H), 7.35 (m, 2H), 7.48 (m, 1H), 7.55 (m, 2H), 7.58 (s, 1H); MS (DCI/NH₃) m/e 335 (M+H)⁺. maleate salt: mp 131-133° C.; Anal. calcd for C₂₄H₂₆N₄O₅.0.30H₂O: C, 63.23; H, 5.88; N, 12.29. Found: C, 63.04; H, 5.74; N, 12.05.

### EXAMPLE 92

### 3-methyl-N-{[4-(2-pyrimidinyl)-1-piperazinyl]methyl}benzamide

The procedure described in Example 91B was followed, substituting 1-(2-pyrimidinyl)piperazine for 1-(2-cyanophenyl)piperazine, to provide the title compound as a colorless oil. ¹H NMR (300 MHz, CDCl₃) δ 2.40 (s, 3H), 2.75 (m, 4H), 3.88 (m, 4H), 4.44 (d, 2H, J=6 Hz), 6.50 (t, 1H, J=4.5 Hz), 6.7 (br s, 1H), 7.32 (d, 2H, J=6 Hz), 7.55 (m, 1H), 7.65 (s, 1H), 8.30 (d, 2H, J=6 Hz); MS (DCI/NH₃) m/e 312 (M+H)⁺.

maleate salt: Obtained as white powder (0.31 g); mp 163-165° C.; Anal. calcd for C₂₁H₂₅N₅O₅: C, 59.01; H, 5.90; N, 16.38. Found: C, 59.05; H, 5.93; N, 16.31.

### EXAMPLE 93

### 3-methyl-N-{[4-(2-pyridinyl)-1-piperazinyl]methyl}benzamide

The procedure described in Example 91B was followed, substituting 1-(2-pyridinyl)piperazine for 1-(2-cyanophenyl)piperazine, to provide the title compound as an off-white residue. ¹H NMR (300 MHz, CDCl₃) δ 2.40 (s, 3H), 2.75 (m, 4H), 3.60 (m, 4H), 4.40 (d, 2H, J=6 Hz), 6.58 (br s, 1H), 6.65 (m, 2H), 7.32 (d, 2H, J=6 Hz), 7.40-7.55 (m, 2H), 7.55 (s, 1H), 8.2 (m, 1H); MS (DCI/NH₃) m/e 311 (M+H)⁺; maleate salt: Off-white solid; mp 141-143° C.; Anal. calcd for C₂₂H₂₆N₄O₅: C, 61.96; H, 6.15; N, 13.14. Found: C, 61.78; H, 6.08; N, 13.09.

### EXAMPLE 94

### 3-methyl-N-[(4-phenyl-1-piperazinyl)methyl]benzamide

The procedure described in Example 91B was followed, substituting 1-phenylpiperazine for 1-(2-cyanophenyl)piperazine, to provide the title compound as a colorless oil. ¹H NMR (300 MHz, CDCl₃) δ 2.40 (s, 3H), 2.82 (m, 4H), 3.21 (m, 4H), 4.44 (d, 2H, J=6 Hz), 6.60 (br s, 1H), 6.82-6.95 (m, 3H), 7.20 (m, 2H), 7.35 (d, 2H, J=6 Hz), 7.58 (m, 1H) 7.63 (s, 1H); MS (DCI/NH₃) m/e 310 (M+H)⁺; maleate salt: Obtained as off-white powder; mp 145-147° C.; Anal. calcd for C₂₃H₂₇N₃O₅: C, 64.93; H, 6.40; N, 9.88. Found: C, 64.83; H, 6.38; N, 9.89.

### EXAMPLE 95

### N-{[4-(2-methoxyphenyl)-1-piperazinyl methyl}-3-methylbenzamide

### EXAMPLE 95A

### [(3-chlorobenzoyl)amino]methyl Acetate

The procedure described in Example 91A was followed, substituting N-(3-chlorobenzoyl)glycine for N-(3-methylbenzoyl)glycine, to provide the title compound.

### EXAMPLE 95B

### N-{4-(2-methoxyphenyl)-1-piperazinyl]methyl}-3-methylbenzamide

The procedure described in Example 91B was followed, substituting the product from Example 95A for the product from Example 91 A and substituting 1-(2-methoxyphenyl)piperazine for 1-(2-cyanophenyl)piperazine, to provide the title compound (1.95 g). ¹H NMR (300 MHz, CDCl₃) δ 2.90 (m, 4H), 3.15 (m, 4H), 3.85 (s, 3H), 4.45 (d, 211, J=6 Hz), 6.55 (br s, 1H), 6.84 (d, 111, J=7.5 Hz), 6.9-7.15 (m, 311,), 7.40 (t, 111, J=7.5 Hz), 7.5 (m, 1H), 7.68 (m, 1H) 7.8 (t, 111, J=3 Hz); MS (DCI/NH₃) m/e 360 (M+H)⁺; maleate salt: brown powder; mp 139-142° C.; Anal. calcd for C₂₃H₂₆ClN₃O₆: C, 57.61; H, 5.55; N, 8.76. Found: C, 57.26; H, 5.65; N, 8.69.

### EXAMPLE 96

### N-{[4-(2-cyanophenyl)-1-piperazinyl]methyl}-2-methylbenzamide

### EXAMPLE 96A

### [(2-methylbenzoyl)amino]methyl acetate

The procedure described in Example 91A was followed, substituting N-(2-methylbenzoyl)glycine for N-(3-methylbenzoyl)glycine, to provide the title compound.

### EXAMPLE 96B

### N-{[4-(2-cyanophenyl)-1-piperazinyl]methyl}-2-methylbenzamide

The procedure described in Example 91B was followed, substituting the product from Example 96A for the product from Example 91A to provide the title compound as an off-white powder. ¹H NMR (300 MHz, CDCl₃) δ 2.50 (s, 3H), 2.90 (t, 4H, J=6 Hz), 3.25 (t, 4H, J=6 Hz), 4.45 (d, 2H, J=6 Hz), 6.18 (br s, 1H), 7.0 (m, 2H), 7.20-7.60 (m, 6H); MS (DCI/NH₃) m/e 335 (M+H)⁺; maleate salt: Yellow powder; mp 62-64° C.; Anal. calcd for C₂₄H₂₆N₄050.20H₂O: C, 63.48; H, 5.86; N, 12.34. Found: C, 63.19; H, 5.77; N, 11.97.

### 405145 EXAMPLE 97

### N-{[4-(2-cyanophenyl)-1-piperazinyl]methyl}-4-methylbenzamide

### EXAMPLE 97A

### [(4-methylbenzoyl)amino]methyl Acetate

The procedure described in Example 91A was followed, substituting N-(4-methylbenzoyl)glycine for N-(3-methylbenzoyl)glycine, to provide the title compound.

### EXAMPLE 97B

### N-{[4-(2-cyanophenyl)-1-piperazinyl]methyl}-4-methylbenzamide

The procedure described in Example 91B was followed, substituting the product from Example 97A for the product from Example 91A to provide the title compound as a colorless oil. ¹H NMR (300 MHz, CDCl₃) δ 2.45 (s, 3H), 2.9 (t, 4H, J=6 Hz), 3.25 (t, 4H, J=6 Hz), 4.45 (d, 2H, J=6 Hz), 6.60 (br s, 1H), 7.0 (m, 2H), 7.25 (m, 2H), 7.45-7.60 (m, 2H), 7.7 (d, 2H, J=9 Hz); MS (DCI/NH₃) m/e 335 (M+H)⁺; maleate salt: Brown powder; Anal. calcd for C₂₄H₂₆N₄O₅: C, 63.99; H, 5.82; N, 12.44. Found: C, 63.71; H, 5.78; N, 12.18.

### EXAMPLE 98

### N-{[4-(3-cyano-2-pyridinyl)-1-piperazinyl]methyl}-3-methylbenzamide

The procedure described in Example 91B was followed, substituting 1-(2-cyanopyridinyl)piperazine for 1-(2-cyanophenyl)piperazine to provide the title compound as a brown oil. ¹H NMR (300 MHz, CHCl₃) δ 2.40 (s, 3H), 2.80 (t, 4H, J=6 Hz), 3.75 (t, 4H, J=6 Hz), 4.40 (d, 2H, J=6 Hz), 6.55 (br s, 1H), 6.75 (dd, 1H, J=12, 6 Hz), 7.32 (d, 2H, J=6 Hz), 7.52-7.65 (m, 2H), 7.75 (dd, 1H, J=7.5, 3 Hz), 8.33 (dd, 1H, J=6, 3 Hz); MS (DCI/NH₃) m/e 336 (M+H)⁺; maleate salt: Pale yellow powder; mp 128-130° C.; Anal. calcd for C₂₃H₂₅N₅O₅:C, 61.19; H, 5.58; N, 15.51. Found: C, 61.46; H, 5.57; N, 15.57.

### EXAMPLE 99

### N-{[4-(3-cyanophenyl)-1-piperazinyl]methyl}-3-methylbenzamide

The procedure described in Example 91B was followed, substituting 1-(3-cyanophenyl)piperazine for 1-(2-cyanophenyl)piperazine to provide the title compound as a glassy solid. ¹H NMR (300 MHz, CDCl₃) δ 2.40 (s, 3H), 2.80 (t, 4H, J=6 Hz), 3.25 (t, 4H, J=6 Hz), 4.40 (d, 2H, J=6 Hz), 6.50 (br s, 1H), 7.1 (m, 3H), 7.35 (m, 3H), 7.55-7.70 (m, 2H); MS (DCI/NH₃) m/e 335 (M+H)⁺; maleate salt: Off-white powder. mp 59-61° C.; Anal. calcd for C₂₄H₂₆N₄O₅: C, 63.99; H, 5.82; N, 12.44. Found: C, 63.76; H, 5.75; N, 12.17.

### EXAMPLE 100

### N-{[4-(3-cyanophenyl)-1-piperazinyl]methyl}-2-methylbenzamide

The procedure described in Example 91B was followed, substituting the product from Example 96A for the product from Example 91A and substituting 1-(3-cyanophenyl)piperazine for 1-(2-cyanophenyl)piperazine to provide the title compound. ¹H NMR (300 MHz, CDCl₃) δ 2.48 (s, 3H), 2.85 (t, 4H, J=6 Hz), 3.25 (t, 4H, J=6 Hz), 4.4 (d, 2H, J=6 Hz), 6.18 (br s, 1H), 7.10 (m, 3H), 7.22 (m, 2H), 7.30-7.45 (m, 3H); MS (DCI/NH₃) m/e 335 (M+H)⁺; maleate salt: Off-white powder; mp 156-159° C.; Anal. calcd for C₂₄H₂₆N₄O₅: C, 63.99; H, 5.82; N, 12.44. Found: C, 63.79; H, 5.67; N, 12.29.

### EXAMPLE 101

### N-{[4-(3-cyano-2-pyridinyl)-1-piperazinyl]methyl}benzamide

### EXAMPLE 101A

### (benzoylamino)methyl Acetate

The procedure described in Example 91A was followed, substituting N-(benzoyl)glycine for N-(3-methylbenzoyl)glycine, to provide the title compound.

### EXAMPLE 101B

### N-{[4-(3-cyano-2-pyridinyl)-1-piperazinyl]methyl}benzamide

The procedure described in Example 91B was followed, substituting the product from Example 101A for the product from Example 91A and substituting 1-(2-cyanopyridinyl)piperazine for 1-(2-cyanophenyl)piperazine to provide the title compound as a colorless oil. ¹H NMR (300 MHz, CDCl₃) δ 2.80 (t, 4H, J=6 Hz), 3.75 (t, 4H, J=6 Hz), 4.40 (d, 2H, J=6 Hz), 6.55 (br s, 1H), 6.75 (dd, 1H, J=12, 6 Hz), 7.40-7.60 (m, 3H), 7.72-7.85 (m, 3H), 8.35 (dd, 1H, 6, 3 Hz); MS (DCI/NH₃) m/e 322 (M+H)⁺.

maleate salt: White solid; mp 133-136° C.; Anal. calcd for C₂₂H₂₃N₅O₅: C, 60.40; H, 5.30; N, 16.01. Found: C, 60.97; H, 5.26; N, 16.31.

### EXAMPLE 102

### N-{[4-(3-cyano-2-pyridinyl)-1-piperazinyl]methyl}-4-methylbenzamide

The procedure described in Example 91B was followed, substituting the product from Example 97A for the product from Example 91A and substituting 1-(2-cyanopyridinyl)piperazine for 1-(2-cyanophenyl)piperazine to provide the title compound as a white solid. ¹H NMR (300 MHz, CDCl₃) δ 2.40 (s, 3H), 2.85 (m, 4H,), 3.75 (m, 4H), 4.43 (m, 2H), 6.75 (m, 1H), 7.22 (m, 2H), 7.70 (d, 2H, J=9 Hz), 7.78 (dd, 1H, J=9, 3 Hz) 8.32 (dd, 1H, J=6, 3 Hz); MS (DCI/NH₃) m/e 336 (M+H)⁺; maleate salt: White solid; mp 134-136° C.; Anal. calcd for C₂₃H₂₅N₅O₅: C, 61.19; H, 5.58; N, 15.51. Found: C, 60.91; H, 5.60; N, 15.60.

### EXAMPLE 103

### N-{[4-(3-cyano-2-pyridinyl)-1-piperazinyl]methyl}-2-methylbenzamide

The procedure described in Example 91B was followed, substituting the product from Example 96A for the product from Example 91A and substituting 1-(2-cyanopyridinyl)piperazine for 1-(2-cyanophenyl)piperazine to provide the title compound as a glassy solid. ¹H NMR (300 MHz, CDCl₃) δ 2.48 (s, 3H), 2.8 (t, 4H, J=6 Hz), 3.75 (t, 4H, J=6 Hz), 4.4 (d, 2H, J=6 Hz), 6.14 (br s, 1H), 6.75 (dd, 1H, J=12, 6 Hz), 7.18-7.41 (m, 4H), 7.78 (dd, 1H, J=9, 3 Hz), 8.35 (dd, 1H, 6, 3 Hz); MS (DCI/NH₃) m/e 336 (M+H)⁺; maleate salt: Off-white powder; mp 124-127° C.; Anal. calcd for C₂₃H₂₅N₅O₅: C, 61.19; H, 5.58; N, 15.51. Found: C, 61.43; H, 5.39; N, 15.81.

### EXAMPLE 104

### N-{[4-(2-pyridinyl)-1-piperazinyl]methyl}benzamide

The procedure described in Example 91B was followed, substituting the product from Example 101A for the product from Example 91A and substituting 1-(2-pyridinyl)piperazine for 1-(2-cyanophenyl)piperazine to provide the title compound as a white sticky residue. ¹H NMR (300 MHz, CDCl₃) δ 2.75 (t, 4H, J=6 Hz), 3.55 (t, 4H, J=6 Hz), 4.4 (d, 2H, J=6 Hz), 6.50 (br s, 1H), 6.65 (m, 2H), 7.40-7.55 (m, 4H), 7.75 (m, 2H), 8.20 (m, 1H); MS (DCI/NH₃) m/e 297 (M+H)⁺; maleate salt: White solid; mp 125-127° C.; Anal. calcd for C₂₁H₂₄N₄O₅: C, 61.15; H, 5.87; N, 13.58. Found: C, 60.86; H, 5.89; N, 13.52.

### EXAMPLE 105

### N-{[4-(2-chlorophenyl)-1-piperazinyl]methyl}benzamide

The procedure described in Example 91B was followed, substituting the product from Example 101A for the product from Example 91A and substituting 1-(2-chlorophenyl)piperazine for 1-(2-cyanophenyl)piperazine to provide the title compound as a colorless oil. ¹H NMR (300 MHz, CDCl₃) δ 2.86 (m, 4H), 3.1 (m, 4H), 4.45 (d, 2H, J=6 Hz), 6.70 (br s, 1H), 7.1 (m, 3H), 7.00 (m, 2H), 7.25 (m, 2H), 7.4-7.26 (m, 2H); MS (DCI/NH₃) nme 330 (M+H)⁺; maleate salt: Tan solid; mp 145-147° C.; Anal. calcd for C₂₂H₂₄ClN₃O₅: C, 59.26; H, 5.43; N, 9.42. Found: C, 58.98; H, 5.34; N, 9.15.

### EXAMPLE 106

### 3-chloro-N-{[4-(2-cyanophenyl)-1-piperazinyl]methyl}benzamide

The procedure described in Example 911B was followed, substituting the product from Example 95A for the product from Example 91A, to provide the title compound as a colorless oil. ¹H NMR (300 MHz, CDCl₃) δ 2.92 (m, 4H), 3.25 (m, 4H), 4.45 (d, 2H, J=6 Hz), 6.75 (br s, 1H), 7.00 (t, 2H, J=6 Hz), 7.35-7.70 (m, 5H), 7.82 (m, 1H); MS (DCI NH₃) m/e 355 (M+H)⁺; maleate salt: White solid; mp 143-146° C.; Anal. calcd for C₂₃H₂₃ClN₄O₅: C, 58.66; H, 4.92; N, 11.90. Found: C, 58.30; H, 5.01; N, 11.67.

### EXAMPLE 107

### 4-chloro-N-{[4-(2-methoxyphenyl)-1-piperazinyl]methyl}benzamide

### EXAMPLE 107A

### [(4-chlorobenzoyl)amino]Methyl Acetate

The procedure described in Example 91A was followed, substituting N-(4-chlorobenzoyl)glycine for N-(3-methylbenzoyl)glycine, to provide the title compound.

### EXAMPLE 107B

### 4-chloro-N-{[4-(2-methoxyphenyl)-1-piperazinyl]methyl}benzamide

The procedure described in Example 91B was followed, substituting the product from Example 107A for the product from Example 91A and substituting 1-(2-methoxyphenyl)piperazine for 1-(2-cyanophenyl)piperazine to provide the title compound as a colorless oil. ¹H NMR (300 MHz, CDCl₃) δ 2.92 (m, 4H), 3.15 (m, 4H), 3.85 (s, 3H), 4.45 (d, 2H, J=6 Hz), 6.7 (br s, 1H), 6.82-7.05 (m, 4H), 7.44 (m, 2H), 7.75 (m, 2H); MS (DCI/NH₃) m/e 360 (M+H)⁺; maleate salt: White solid; mp 145-147° C.; Anal. calcd for C₂₃H₂₆ClN₃O₆: C, 58.04; H, 5.51; N, 8.83. Found: C, 58.24; H, 5.18; N, 8.83.

### EXAMPLE 108

### 2-chloro-N-{[4-(3-cyano-2-pyridinyl)-1-piperazinyl]methyl}benzamide

### EXAMPLE 108A

### [(2-chlorobenzoyl)amino]methyl Acetate

The procedure described in Example 91A was followed, substituting N-(2-chlorobenzoyl)glycine for N-(3-methylbenzoyl)glycine, to provide the title compound.

### EXAMPLE 108B 2-chloro-N-{[4-(3-cyano-2-pyridinyl)-1-piperazinyl]methyl}benzamide

The procedure described in Example 91B was followed, substituting the product from Example 108A for the product from Example 91A and substituting 1-(2-cyanopyridinyl)piperazine for 1-(2-cyanophenyl)piperazine to provide the title compound as a yellow oil. ¹H NMR (300 MHz, CDCl₃) δ 2.75 (t, 4H, J=6 Hz), 3.55 (t, 4H, J=6 Hz), 4.4 (d, 2H, J=6 Hz), 6.50 (br s, 1H), 6.65 (m, 2H), 7.40-7.55 (m, 4H), 7.75 (m, 2H), 8.20 (m, 1H); MS (DCI/NH₃) m/e 356 (M+H)⁺; maleate salt: White solid; mp 137-139° C.; Anal. calcd for C₂₂H₂₂ClN₅O₅: C, 55.99; H, 4.70; N, 14.84. Found: C, 55.76; H, 4.74; N, 14.60.

### EXAMPLE 109

### N-{[4-(3-cyano-2-pyridinyl)-1-piperazinyl]meth}-2-(trifluoromethyl)benzamide

### EXAMPLE 109A

### {[2-(trifluoromethyl)benzoyl]amino}methyl acetate

The procedure described in Example 91A was followed, substituting N-[2-(trifluoromethyl)benzoyl] glycine for N-(3-methylbenzoyl)glycine, to provide the title compound.

### EXAMPLE 109B

### N-{[4-(3-cyano-2-pyridinyl)-1-piperazinyl]methyl}-2-(trifluoromethyl)benzamide

The procedure described in Example 91B was followed, substituting the product from Example 109A for the product from Example 91A and substituting 1-(2-cyanopyridinyl)piperazine for 1-(2-cyanophenyl)piperazine to provide the title compound as a colorless oil. ¹H NMR (300 MHz, CDCl₃) δ 2.90 (m, 4H), 3.80 (m, 4H), 4.45 (d, 2H, J=6 Hz), 6.80 (dd, 1H, J=12, 6 Hz), 7.55-7.80 (m, 5H), 8.35 (dd, 1H, J=6, 3 Hz), 11.00 (br s, 1H); MS (DCI/NH₃) m/e 390 (M+H)⁺. maleate salt: Hygroscopic white solid.

### EXAMPLE 110

### N-{[4-(2-cyanophenyl)-1-piperazinyl]methyl}benzamide

The procedure described in Example 91B was followed, substituting the product from Example 101A for the product from Example 91A, to provide the title compound as a yellow oil. ¹H NMR (300 MHz, DMSO-d₆) δ 2.71 (m, 4H), 3.15 (m, 4H), 4.22 (d, 2H, J=6.1 Hz), 7.08 (dd, 1H, J=7.8, 7.8 Hz), 7.15 (d, 1H, J=8.5 Hz), 7.55 (m, 4H), 7.68 (dd, 1H, J=7.4, 1.3 Hz), 7.90 (m, 2H), 8.95 (t, 1H, J=6.1 Hz); MS (DCI/NH₃) m/e 321 (M+H)⁺; maleate salt: Tan solid, mp 148-150° C.; Anal. calcd for C₁₉H₂₀N₄O_{1.0} C₄H₄O₄: C, 63.29; H, 5.54; N, 12.84. Found: C, 63.03; H, 5.47; N, 12.79.

### EXAMPLE 111

### N-{[4-(2-methoxyphenyl)-1-piperidinyl]methyl}-3-methylbenzamide

4-(2-Methoxyphenyl)piperidine (286 mg, 1.5 mmol), the product from Example 91A (310 mg, 1 mmol), and triethylamine (0.42 mL, 3 mmol) were combined in acetonitrile (8 mL) and stirred at room temperature for 18 hours. The reaction mixture was concentrated under reducec pressure and the residue was purified by flash chromatography on silica gel (elution with dichloromethane:methanol 9.5:0.5) to provide the title compound (285 mg, 56.2% yield). ¹H NMR (300 MHz, DMSO-d₆) δ 1.65 (m, 4H), 2.31 (m, 2H), 2.37 (s, 3H), 2.79 (m, 1H), 2.93 (m, 2H), 3.75 (s, 3H), 4.15 (d, 2H, J=6 Hz), 6.90 (m, 2H), 7.15 (m, 2H), 7.36 (m, 2H), 7.68 (m, 2H), 8.69 (t, 1H, J=6 Hz); MS (DCI/NH₃) m/e 339 (M+H)⁺; Anal. calcd for C₂₁H₂₆N₂O₂.0.15H₂O: C, 73.94; H, 7.77; N, 8.21. Found: C, 73.56, H, 7.72, N, 8.15.

### EXAMPLE 112

### 3-methyl-N-{[4-(2-pyridinyl)-1-piperidinyl]methyl}benzamide

The procedure described in Example 111 was followed, substituting the product from Example 36C for 4-(2-methoxyphenyl)piperidine, to provide the title compound (480 mg, 64% yield). ¹H NMR (300 MHz, DMSO-d₆) δ 1.75 (m, 4H), 2.31 (m, 2H), 2.36 (s, 3H), 2.59 (m, 1H), 2.95 (m, 2H), 4.17 (d, 2H, J=6 Hz), 7.18 (m, 1H), 7.25 (d, 1H, J=6 Hz), 7.35 (m, 2H), 7.69 (m, 3H), 8.48 (m, 1H), 8.71 (m, 1H); MS (DCI/NH₃) m/e 310 (M+H)⁺. Anal. calcd for C₁₉H₂₃N_{3.}0.0.25H₂O: C, 72.70; H, 7.50; N, 13.39. Found: C, 72.60, H, 7.50, N, 13.21.

### EXAMPLE 113

### 3-methyl-N-[(4-phenyl-3,6-dihydro-1(2H)-pyridinyl)methyl]benzamide

The procedure described in Example 111 was followed, substituting 4-phenyl-1,2,3,6-tetrahydropyridine for 4-(2-methoxyphenyl)piperidine, to provide the title compound (196 mg, 64% yield). ¹H NMR (300 MHz, DMSO-d₆) δ 2.35 (s, 3H), 2.76 (t, 2H, J=6 Hz), 3.24 (d, 2H), 4.25 (d, 4H, J=9 Hz), 6.16 (m, 1H), 7.22 (t, 1H, J=6 Hz), 7.32 (m, 4H), 7.40 (m, 2H), 7.66 (m, 2H), 8.75 (t, 1H, J=6 Hz); MS (DCI/NH₃) m/e 307 (M+H)⁺. Anal. calcd for C₂₀H₂₂N₂O_{0.10}H₂O: C, 77.94; H, 7.26; N, 9.09. Found: C, 77.64, H, 7.34, N, 8.86.

### EXAMPLE 114

### N-(3′.6′-dihydro-2,4′-bipyridin-1′(2′H)-ylmethyl)-3-methylbenzamide

The procedure described in Example 111 was followed, substituting 1′,2′,3′,6′-tetrahydro-2,4′-bipyridine hydrochloride for 4-(2-methoxyphenyl)piperidine, to provide the title compound (310 mg, 81.5% yield). ¹H NMR (300 MHz, DMSO-d₆) δ 2.35 (s, 3H), 2.58 (m, 2H), 2.76 (t, 2H, J=6 Hz), 3.29 (m, 2H), 4.27 (d, 2H, J=6 Hz), 6.70 (m, 1H), 7.22 (m, 1H), 7.35 (d, 2H, J=6 Hz), 7.51 (d, 1H, J=9 Hz), 7.70 (m, 3H), 8.51 (m, 1H), 8.76 (m, 1H); MS (DCI/NH₃) m/e 308 (M+H)⁺.

### EXAMPLE 115

### N-(3′,6′-dihydro-2,4′-bipyridin-1′(2′H)-ylmethyl)-3-methoxybenzamide

3-Methoxybenzamide (1.13 g, 7.5 mmol), K₂CO₃ (345 mg, 2.5 mmol), paraformaldehyde (0.5 g, 16 mmol), and 1′,2′,3′,6′-tetrahydro-2,4′-bipyridine hydrochloride (393 mg, 2 mmol) were combined in ethanol (25 ml) and refluxed for 18 hours. The reaction mixture was allowed to cool to room temperature and concentrated under reduced pressure. The residue was partitioned between ethyl acetate (80 mL) and water (80 mL). The organic layer was washed with brine (2×50 mL), dried over MgSO₄, filtered, and the filtrate concentrated under reduced pressure. The residue was purified by flash chromatography on silica gel (elution with ethyl acetate:ethanol, 9.0:1.0) to provide the title compound (180 mg, 49% yield). ¹H NMR (300 MHz, DMSO-d₆) δ 2.58 (m, 2H), 2.76 (t, 2H, J=6 Hz), 3.29 (m, 2H), 3.80 (s, 3H), 4.27 (d, 2H, J=6 Hz), 6.70 (m, 1H), 7.09 (m, 1H), 7.22 (m, 1H), 7.42 (m, 4H), 7.72 (m, 1H), 8.51 (m, 1H), 8.83 (t, 1H, J=6 Hz); MS (DCI/NH₃) m/e 324 (M+H)⁺. Anal. calcd for C₁₉H₂₁N₃O₂₀.60H₂O: C, 68,28; H, 6.70; N, 12.57. Found: C, 68.19, H, 6.84, N, 11.77.

### EXAMPLE 116

### N-(3′,6′-dihydro-2,4′-bipyridin-1′(2′H)-ylmethyl)-3-fluorobenzamide

The procedure described in Example 115 was followed, substituting 3-fluorobenzamide for 3-methoxybenzamide, to provide the title compound (260 mg, 42.6% yield). ¹H NMR (300 MHz, DMSO-d₆) δ 2.58 (m, 2H), 2.76 (t, 2H, J=6 Hz), 3.29 (m, 2H), 4.27 (d, J=6 Hz, 2H), 6.70 (m, 1H), 7.21 (m, 1H), 7.39 (m, 1H), 7.51 (m, 2H), 7.72 (m, 3H), 8.51 (m, 1H), 8.93 (t, 1H, J=6 F=Hz); MS (DCI/NH₃) m/e 312 (M+H)⁺.

### EXAMPLE 117

### N-(3′,6′-dihydro-2,4′-bipyridin-1′(2′H)-ylmethyl)-3,5-difluorobenzamide

The procedure described in Example 115 was followed, substituting 3,5-difluorobenzamide for 3-methoxybenzamide, to provide the title compound (140 mg, 21% yield). ¹H NMR (300 MHz, DMSO-d₆) δ 2.58 (m, 2H), 2.76 (t, 2H, J=6 Hz), 3.29 (m, 2H), 4.27 (d, 2H, J=6 Hz), 6.70 (m, 1H), 7.21 (m, 1H), 7.51 (m, 2H), 7.60 (m, 2H), 7.75 (m, 1H), 8.51 (m, 1H), 9.01 (t, 1H, J=6 Hz); MS (DCI/NH₃) m/e 330 (M+H)⁺. Anal. calcd for C₁₈H₁₇N₃OF₂₀.70H₂O: C, 63.22; H, 5.42; N, 12.29. Found: C, 62.76, H, 5.02, N, 12.09.

### EXAMPLE 118

### 2-[4-(3-cyano-2-pyridinyl]-1-piperazinyl]-N-3-pyridinylacetamide

The procedure described in Example 8 was followed, substituting 2-chloro-N-3-pyridinylacetamide (Abdel Rahman, A. E.; et al. J. Ind. Chem. Soc. 1981, 58, 171-173) for N-chloroacetyl-3-nitroaniline, to provide the title compound in 13% yield. The free base was treated with maleic acid to provide the maleate salt as a yellow solid. ¹H NMR (300 MHz, MeOH-d4) δ 8.43 (dd,1H, J=4.7, 1.7 Hz), 8.33 (br d, 1H, J=4.1 Hz), 8.17 (ddd, 1H, J=8.5, 2.4, 1.4 Hz), 8.00 (dd, 1H, J=7.8, 2.0 Hz), 7.47 (dd, 1H, J=8.5, 5.1 Hz), 7.00 (dd, 1H, J=7.8, 5.1 Hz), 6.27 (s, 2H), 3.88 (m, 6H), 3.28 (m, 4H); MS (DCI/NH₃) m/e 323 (M+H)⁺; Anal. calcd for C₁₇H₁₈N₆O.1.2 C₄H₄O⁴.0.40H₂O: C, 55.85; H, 5.07; N, 17.92; Found: C, 55.66; H, 5.14; N, 17.91.

### EXAMPLE 119

### 2-(1-{2-[(3-methylphenyl)amino]-2-oxoethyl}piperidin-4-yl)pyridinium N-oxide

### EXAMPLE 119A

### 2-piperidin-4-ylpyridinium N-oxide hydrochloride

2-[1-(tert-butoxycarbonyl)piperidin-4-yl]pyridinium N-oxide (1.24 g, 4.15 mmol) in dichloromethane (30 mL) was cooled to 0° C. and treated with m-chloroperbenzoic acid 77% (1.4 g, 8.3 mmol). After stirring at 0° C. for 30 minutes, the mixture was allowed to warm to room temperature and stir an additional for 2 hrs. The mixture was diluted with methylene chloride (50 mL), washed with saturated NaHCO₃, brine, dried over MgSO₄, filtered, and the filtrate concentrated under reduced pressure to provide white solid. The white solid was dissolved in ethyl acetate (50 mL) and cooled to −78° C. HCl gas was bubbled through the reaction mixture for 15 minutes and the mixture was allowed to warm to room temperature. The mixture was filtered and the filter cake washed with ethyl acetate and then dried under high vaccum to provide the title compound (0.85 g, 96% yield). ¹H NMR (300 MHz, DMSO-d₆) δ 1.82 (m, 2H), 2.10 (m, 2H), 3.06 (m, 2H), 3.36 (m, 2H), 3.58 (m, 1H), 7.45 (m, 3H), 8.39 (d, J=9 Hz, 1H), 9.04 (bs, 1H); MS (DCI/NH₃) m/z 179 (M+H)⁺.

### EXAMPLE 119B

### 2-(1-{2-[(3-methylphenyl)amino]-2-oxoethyl}piperidin-4-yl)pyridinium N-oxide

The procedure described in Example 36D was followed, substituting the product from Example 119A for the product from Example 36C, to provide the title compound (159 mg, 48.8% yield). ¹H NMR (300 MHz, DMSO-d₆) δ 1.89 (m, 2H), 1.91(mp, 2H), 2.30 (m, 5H), 2.99 (m, 2H), 3.14 (s, 2H), 3.25 (m, 1H), 6.88 (d, J=7.5 Hz, 1H), 7.19 (t, J=7.5 Hz, 1H), 7.31 (m, 2H), 7.45 (m, 2H), 8.24 (m, 1H), 9.6 (bs, 1H); MS (DC₁—NH₃) m/z 310 (M+H)⁺. The free base (156.7 mg) in ethanol (20 mL) was treated with maleic acid (55.93 mg) and the solution was stirred for 10 minutes, concentrated under reduced pressure to provide the maleate salt as an off white solid (212.6 mg). ¹H NMR (300 MHz, DMSO-d₆) δ 1.91(m, 2H), 2.15 (m, 2H), 2.29 (s, 3H), 3.30 (m, 4H), 3.50 (m, 2H), 4.02 (m, 1H), 6.04 (s, 2H), 6.95 (d, J=7.5 Hz, 1H), 7.23 (t, J=7.5 Hz, 1H), 7.39 (m, 5H), 8.29 (m, 1H), 10.36 (bs, 1H); MS (DC₁—NH₃) m/z 310 (M+H)⁺; Analysis calculated for 0.25H₂O.C₂₃H₂₇N₃O₆: C, 61.94; H, 6.22; N, 9.42; Found: C, 61.56, H, 6.21, N, 8.99.

### In Vitro Data

### Functional Activity of D

Efficacies and potencies of compounds of the present invention at the human D₄ receptor were determined using a stable cell line containing the human D₄.₄ receptor and a chimeric G protein in HEK-293 cells. This cell line allows a robust calcium signal detectable using a calcium fluorescent dye and a fluorescent imaging plate reader (FLIPR) (Coward et al., Anal. Biochem. 270: 242-248, 1999). Cells were plated (20000/well) into 96 well dishes and cultured for 48 hours. Media is removed, Fluo-4 dye added and cells incubated 1 hour at room temperature. Cells are washed with phosphate buffered saline to remove excess dye and the compounds to be tested are added to the wells and signal measured in FLIPR. Percent efficacy is the maximum response produced by the compound in relation to the maximum effect of 10 μM dopamine. The EC₅₀ is the effective concentration of the compound that causes 50% of the compound's maximum response.

Chimeric G-proteins allow a high-throughput signaling assay of Gi-coupled receptors, P. Coward, S. Chan, H. Wada, G. Humpries and B. Conklin, Analytical Biochemistry 270, 242-248 (1999).

Representative compounds of the present invention exhibited EC₅₀, in the range of 1 nM to 1600 nM.

### In Vivo Data

### Rat Penile Erection Model

Wistar rats were used as a primary animal model to study penile erection in vivo. All experiments were carried out between 9:00 AM and 3:00 PM in a diffusely illuminated testing room with a red light. Animals were weighed and allowed to adapt to the testing room for 60 minutes before the beginning of experiments. Rats were placed individually in a transparent cage (20×30×30 cm) after drug injection. The number of penile erections were recorded by direct observation for a period of 60 minutes after drug dosing, and the number of animals exhibiting 1 or more erections was expressed as incidence (%). (L)-Ascorbic acid in saline (1 mg/mL) was used as vehicle and apomorphine was used as a positive control at a dose of 0.1 μmol/kg.

Representative compounds of the present invention induced a minimum of 30% incidence of penile erections in rats after subcutaneous administration at doses of 0.003 μmol/kg to 3 μmol/kg.

The in vitro and in vivo data demonstrates that compounds of the present invention are dopamine D₄ receptor agonists that induce penile erections in mammals.

Compounds of the present invention are dopamine D₄ receptor agonists and are useful for the treatment of male sexual dysfunction, female sexual dysfunction, attention deficit hyperactivity disorder, Alzheimer's disease, drug abuse, Parkinson's disease, anxiety, schizophrenia, mood disorders and depression, as described in: The dopamine D₄ receptor: a controversial therapeutic target, N. J. Hrib, Drugs of the future 25:587-611 (2000); Dopamine and sexual behavior, M. Melis and A. Argiolas, Neuroscience and Biobehavioral Reviews 19:19-38 (1995); and Dopamine receptors: from structure to function, C. Missale, S. R. Nash, S. Robinson, M. Jabber and M. Caron, Physiological Reviews 78: 189-225 (1998).

Compounds of the present invention are dopamine D₄ receptor agonists and are useful for the treatment of cardiovascular disorders. Dopamine and dopaminergic agents have been reported to exert pharmacologically significant cardiovascular effects on blood pressure and heart rate and are useful in the treatment of cardiovascular disorders, as described in: Chen F F, and Lin M T, Effects of dopamine, apomorphine gamma-hydroxybutyric acid, haloperidol, and pimozide on reflex bradycardia in rats, Journal of Pharmacology and Experimental Therapeutics (1980) 214: 427-432; and it has been reported that primate data support the potential clinical utility of dopamine receptor agonists in treating cardiovascular disease, as described in: Hahn, R A and MacDonald B R, Primate cardiovascular responses meditated by dopaminine receptors: effects of N,N-dipropyldopamine and LY171555, Journal of Pharmacology and Experimental Therapeutics (1984) 229: 132-138.

Compounds of the present invention are dopamine D₄ receptor agonists and are useful for the treatment of inflammation. Dopaminergic agents can exert anti-inflammatory effects and are useful for the treatment of diseases where inflammation plays a deleterious role, as described in: Bendele A M, Spaethe S M, Benslay D N, and Bryant H U, Anti-inflammatory activity of pergolide, a dopamine receptor agonist, in Journal of Pharmacology of Pharmacology and Experimental Therapeutics (1991) 259 169-175. Dopaminergic agents can also be of utility in the treatment of cancers, as described in: Lissoni P, Mandala M, Giani L, Malugani F, Secondino S, Zonato S, Rocco F, Gardani G, Efficacy of Bromocriptine in the Treatment of Metastatic Breast Cancer and Prostate Cancer-related Hyperprolactinemia, Neuroendocrinology Letters (2000) 21 405-408.

The term agonist, as used herein, means a compound of the present invention that exhibits 30% or greater efficacy in the in vitro assay described herein.

The term “pharmaceutically acceptable carrier” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The present invention provides pharmaceutical compositions which comprise compounds of the present invention formulated together with one or more non-toxic pharmaceutically acceptable carriers.

Dosage forms for topical administration of a compound of the present invention include powders, sprays, ointments and inhalants. The active compound is mixed under sterile conditions with a pharmaceutically acceptable carrier and any needed preservatives, buffers or propellants which can be required. Opthalmic formulations, eye ointments, powders and solutions are also contemplated as being within the scope of this invention.

When used in the above or other treatments, a therapeutically effective amount of one of the compounds of the present invention can be employed in pure form or, where such forms exist, in pharmaceutically acceptable salt, ester, amide, or prodrug form. Alternatively, the compound can be administered as a pharmaceutical composition containing the compound of interest in combination with one or more pharmaceutically acceptable carriers. The phrase “therapeutically effective amount” of the compound of the present invention means a sufficient amount of the compound to treat disorders, at a reasonable benefit/risk ratio applicable to any medical treatment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The total daily dose of the compounds of the present invention administered to a mammal, and particularly a human, may range from about 0.001 to about 30 mg/kg/day. For purposes of oral administration, more preferable doses can be in the range of from 0.01 to about 10 mg/kg/day. If desired, the effective daily dose can be divided into multiple doses for purposes of administration; consequently, single dose compositions may contain such amounts or submultiples thereof to make up the daily dose.

The pharmaceutical compositions of this invention can be administered to humans and other mammals orally, rectally, parenterally, intracisternally, intravaginally, intraperitoneally, topically (as by powders, ointments or drops), bucally or as an oral or nasal spray. The term “parenterally” as used herein, refers to modes of administration which include intravenous, intramuscular, intraperitoneal, intrasternal, subcutaneous and intraarticular injection and infusion.

Pharmaceutical compositions of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), vegetable oils (such as olive oil), injectable organic esters (such as ethyl oleate) and suitable mixtures thereof. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents, for example, paraben, chlorobutanol, phenol sorbic acid and the like. It may also be desirable to include isotonic agents such as sugars, sodium chloride and the like. Prolonged absorption of the injectable pharmaceutical form can be brought about by the inclusion of agents which delay absorption such as aluminum monostearate and gelatin.

In some cases, in order to prolong the effect of the drug, it is desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Ejectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Solid dosage forms for oral administration include capsules, tablets, pills, powders and granules. In such solid dosage forms, the active compound may be mixed with at least one inert, pharmaceutically acceptable excipient or carrier, such as sodium citrate or dicalcium phosphate and/or a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills and granules can be prepared with coatings and shells such as enteric coatings and other coatings well-known in the pharmaceutical formulating art. They may optionally contain opacifying agents and may also be of a composition such that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor and sesame oils), glycerol, tetrahydrofurfuryl alcohol, polyethylene glycols and fatty acid esters of sorbitan and mixtures thereof.

Suspensions, in addition to the active compounds, may contain agents as, for example, ethoxylated isostearyl alcohols, polyoxyethylene sorbitol and sorbitan esters, microcrystalline cellulose, aluminum metahydroxide, bentonite, agar-agar, tragacanth and mixtures thereof.

Compositions for rectal or vaginal administration are preferably suppositories which can be prepared by mixing the compounds of the present invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolizable lipid capable of forming liposomes can be used. The present compositions in liposome form can contain, in addition to a compound of the present invention, stabilizers, preservatives, excipients and the like. The preferred lipids are natural and synthetic phospholipids and phosphatidyl cholines (lecithins) used separately or together.

Methods to form liposomes are known in the art. See, for example, Prescott, Ed., Methods in Cell Biology, Volume XIV, Academic Press, New York, N.Y. (1976), p. 33 et seq.

The present invention contemplates pharmaceutically active compounds either chemically synthesized or formed by in vivo biotransformation to compounds of formula (I).

The compounds of the invention can exist in unsolvated as well as solvated forms, including hydrated forms, such as hemi-hydrates. In general, the solvated forms, with pharmaceutically acceptable solvents such as water and ethanol among others are equivalent to the unsolvated forms for the purposes of the invention.

The term “pharmaceutically acceptable salt, ester, amide, and prodrug” as used herein, refers to carboxylate salts, amino acid addition salts, zwitterions, esters, amides, and prodrugs of compounds of formula (I) which are within the scope of sound medical judgement, suitable for use in contact with the tissues of humans and lower animals without undue toxicity, irritation, allergic response, and the like, are commensurate with a reasonable benefit/risk ratio, and are effective for their intended use.

The compounds of the present invention can be used in the form of pharmaceutically acceptable salts derived from inorganic or organic acids. The term “pharmaceutically acceptable salt” means those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. The salts can be prepared in situ during the final isolation and purification of the compounds of the present invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, sulfate, bis(tartrate), tartrate, (L) tartrate, bis((L) tartrate), (D) tartrate, bis((L) tartrate), (DL) tartrate, bis((DL) tartrate), meso-tartrate, bis(meso tartrate), thiocyanate, phosphate, glutamate, bicarbonate, p-toluenesulfonate and undecanoate. Examples of acids which can be employed to form pharmaceutically acceptable acid addition salts include such inorganic acids as hydrochloric acid, hydrobromic acid, sulphuric acid and phosphoric acid and such organic acids as maleic acid, fumaric acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediaamine, ethanolamine, diethanolaamine, piperidine, piperazine and the like. Preferred salts of the compounds of the present invention include phosphate, tris and acetate.

The term “pharmaceutically acceptable prodrug” or “prodrug” as used herein, represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to compounds of formula (I), for example, by hydrolysis in blood.

The term “pharmaceutically acceptable ester” or “ester” as used herein, refers to esters of compounds of the present invention which hydrolyze in vivo and include those that break down readily in the human body to leave the parent compound or a salt thereof. Examples of pharmaceutically acceptable, non-toxic esters of the present invention include C₁-to-C₆ alkyl esters and C₅-to-C₇ cycloalkyl esters, although C₁-to-C₄ alkyl esters are preferred. Esters of the compounds of formula (I) may be prepared according to conventional methods.

The term “pharmaceutically acceptable amide” or “amide” as used herein, refers to non-toxic amides of the present invention derived from ammonia, primary C₁-to-C₆ alkyl amines and secondary C₁-to-C₆ dialkyl amines. In the case of secondary amines, the amine may also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of formula (I) may be prepared according to conventional methods.

What is claimed is:

1. A method of treating sexual dysfunction in a mammal comprising administering to said mammal in need of such treatment a therapeutically effective amount of a compound of formula (I) 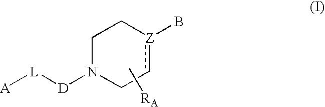
or a pharmaceutically acceptable salt, ester, amide, or prodrug thereof, wherein
A is selected from the group consisting of aryl, arylalkyl, cycloalkyl, cycloalkylalkyl, heterocycle, and heterocyclealkyl;
L is selected from the group consisting of —N(R₇)C(O)— and —C(O)N(R₇)— wherein the left end of said —N(R₇)C(O)— or —C(O)N(R₇)— is attached to A and the right end is attached to D;
D is selected from the group consisting of alkylene, fluoroalkylene, and hydroxyalkylene;
R_{A} is selected from the group consisting of hydrogen and alkyl;
Z is selected from the group consisting of N, C and CH;
— is a bond when Z is C and — is absent when Z is N or CH;
B is selected from the group consisting of 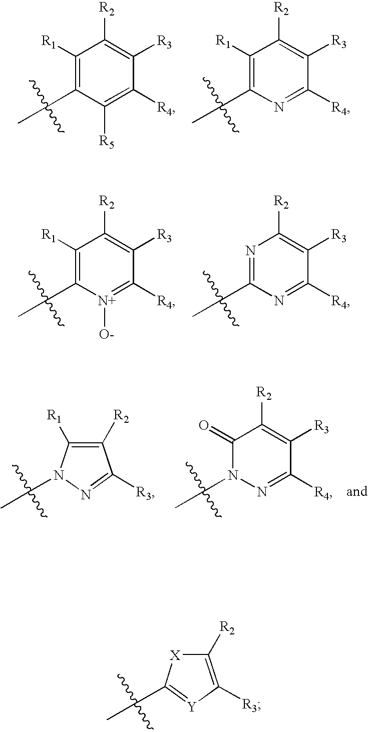
R₁, R₂, R₃, R₄ and R₅ are each independently selected from the group consisting of hydrogen, alkoxy, alkenyl, alkyl, alkylsulfinyl, alkylsulfonyl, alkylthio, alkynyl, alkoxycarbonyl, alkylcarbonyl, alkylcarbonyloxy, carboxy, cyano, formyl, halogen, haloalkoxy, haloalkyl, hydroxy, hydroxyalkyl, mercapto, nitro, —NZ₁Z₂, (NZ₃Z₄)carbonyl, and (NZ₃Z₄)sulfonyl;
Z₁ and Z₂ are each independently selected from the group consisting of hydrogen, alkyl, alkylcarbonyl, alkylsulfonyl, aryl, arylalkyl, arylalkylsulfonyl, arylsulfonyl, and formyl;
Z₃ and Z₄ are each independently selected from the group consisting of hydrogen, alkyl, aryl, and arylalkyl;
X is selected from the group consisting of N(R₆), O and S;
Y is selected from the group consisting of C(R₄) and N;
R₆ is selected from the group consisting of hydrogen and alkyl; and
R₇ is selected from the group consisting of hydrogen and alkyl.

2. The method according to claim 1 wherein
A is aryl;
B is 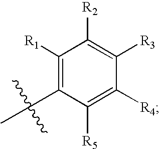
Z is N;
— is absent; and
L is —N(R₇)C(O)—.

3. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 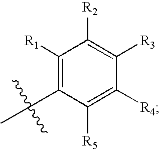
R₁ is selected from the group consisting of hydrogen, alkoxy, alkyl, cyano, halogen, and nitro;
R₂ is selected from the group consisting of hydrogen, cyano, and halogen;
R₃, R₄, and R₅ are hydrogen;
Z is N;
— is absent;
D is —CH₂—; and
L is —N(R₇)C(O)—.

4. The method according to claim 3 wherein the compound of formula (I) is selected from the group consisting of
2-[4-(2-methoxyphenyl)-1-piperazinyl]-N-(3-methylphenyl)acetamide;
2-[4-(2-cyanophenyl)-1-piperazinyl]-N-(3-methylphenyl)acetamide;
N-(3-methylphenyl)-2-[4-(2-methylphenyl)-1-piperazinyl]acetamide;
N-(3-methylphenyl)-2-[4-(2-nitrophenyl)-1-piperazinyl]acetamide;
N-(3-methylphenyl)-2-(4-phenyl-.-piperazinyl)acetamide;
N-(4-bromo-3-methylphenyl)-2-[4-(2-cyanophenyl)-1-piperazinyl]acetamide; and
2-[4-(2-cyanophenyl)-1-piperazinyl]-N-phenylacetamide.

5. The method according to claim 1 wherein
A is aryl;
B is 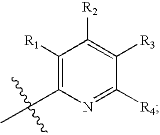
Z is N;
— is absent; and
L is —N(R₇)C(O)—.

6. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 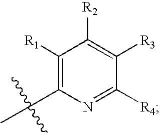
R₁ is selected from the group consisting of hydrogen, alkyl, cyano, and halogen;
R₂, R₃, and R₄ are hydrogen;
Z is N;
— is absent;
D is —CH₂—; and
L is —N(R₇)C(O)—.

7. The method according to claim 6 wherein the compound of formula (I) is selected from the group consisting of
N-(3-methylphenyl)-2-[4-(2-pyridinyl)-1-piperazinyl]acetamide;
2-[4-(3-cyano-2-pyridinyl)-1-piperazinyl]-N-(3-methylphenyl)acetamide;
2-[4-(3-cyano-2-pyridinyl)-piperazinyl]-N-(3-nitrophenyl)acetamide;
2-[4-(3-cyano-2-pyridinyl)-1-piperazinyl]-N-[3-(trifluoromethyl)phenyl]acetamide;
N-(3-cyanophenyl)-2-[4-(3-cyano-2-pyridinyl)-piperazinyl]acetamide;
2-[4-(3-cyano-2-pyridinyl)₅-piperazinyl]-N-phenylacetamide;
2-[4-(3-cyano-2-pyridinyl)-1-piperazinyl]-N-(4-fluorophenyl)acetamide;
2-[4-(3-cyano-2-pyridinyl)-1-piperazinyl]-N-(3,5-dimethylphenyl)acetamide;
2-[4-(3-cyano-2-pyridinyl)-1-piperazinyl]-N-(2,3-dimethylphenyl)acetamide;
2-[4-(3-cyano-2-pyridinyl)-1-piperazinyl]-N-(2-methylphenyl)acetamide;
2-[4-(3-cyano-2-pyridinyl)-1-piperazinyl]-N-(2,5-dimethylphenyl)acetamide;
N-(3-chlorophenyl)-2-[4-(3-cyano-2-pyridinyl) 1-piperazinyl]acetamide;
2-[4-(3-cyano-2-pyridinyl)-1-piperazinyl]acetamide;
2-[4-(3-cyano-2-pyridinyl)-1-piperazinyl]-N-(3,4,5-trimethoxyphenyl)acetamide;
2-[4-(3-cyano-2-pyridinyl)-1-piperazinyl]-N-[4-fluoro-3-(trifluoromethyl)phenyl]acetamide;
2-[4-(3-cyano-2-pyridinyl)-1-piperazinyl]-N-[3-fluoro-5-(trifluoromethyl)phenyl]acetamide;
2-[4-(3-cyano-2-pyridinyl)-1-piperazinyl]-N-[2-fluoro-5-(trifluoromethyl)phenyl]acetamide;
2-[4-(3-cyano-2-pyridinyl)-1-piperazinyl]-N-[2-fluoro-3-(trifluoromethyl)phenyl]acetamide;
2-[4-(3-cyano-2-pyridinyl)-1-piperazinyl]-N-(4-fluoro-3-methylphenyl)acetamide;
2-[4-(3-cyano-2-pyridinyl)-1-piperazinyl]-N-(2-fluorophenyl)acetamide;
2-[4-(3-cyano-2-pyridinyl)-1-piperazinyl]-N-(2-methoxyphenyl)acetamide;
2-[4-(3-cyano-2-pyridinyl)-1-piperazinyl]-N-(2-nitrophenyl)acetamide;
2-[4-(3-cyano-2-pyridinyl)-1-piperazinyl]-N-[2-(trifluoromethyl)phenyl]acetamide;
N-phenyl-2-[4-(2-pyridinyl)-1-piperazinyl]acetamide; and
2-[4-(3-cyano-2-pyridinyl)-1-piperazinyl]-N-(4-methylphenyl)acetamide.

8. The method according to claim 1 wherein
A is aryl wherein the aryl is tetrahydronaphthalenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 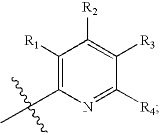
R₁ is selected from the group consisting of hydrogen, alkyl, cyano, and halogen;
R₂, R₃, and R₄ are hydrogen;
Z is N;
— is absent;
D is —CH₂—; and
L is —N(R₇)C(O)—.

9. The method according to claim 1 wherein
A is aryl;
B is 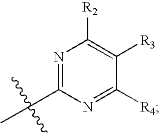
Z is N;
— is absent; and
L is —N(R₇)C(O)—.

10. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 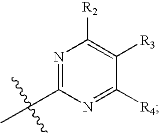
R₂, R₃, and R are hydrogen;
Z is N;
— is absent;
D is —CH₂—; and
L is —N(R₇)C(O)—.

11. The method according to claim 10 wherein the compound of formula (I) is N-(3-methylphenyl)-2-[4-(2-pyrimidinyl)-1-piperazinyl]acetamide.

12. The method according to claim 1 wherein
A is aryl;
B is 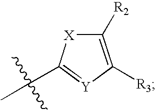
Z is N;
— is absent; and
L is —N(R₇)C(O)—.

13. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 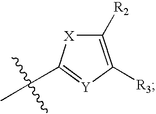
R₂ and R₃ are hydrogen;
X is S;
Y is N;
Z is N;
— is absent;
D is —CH₂—; and
L is —N(R₇)C(O)—.

14. The method according to claim 13 wherein the compound of formula (I) is N-(3-methylphenyl)-2-[4-(1,3-thiazol-2-yl)-1-piperazinyl]acetamide.

15. The method according to claim 1 wherein
A is heterocycle;
B is 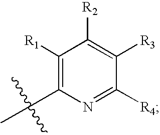
Z is N;
— is absent; and
L is —N(R₇)C(O)—.

16. The method according to claim 1 wherein
A is heterocycle wherein the heterocycle is selected from the group consisting of furyl, imidazolyl, 1,3-oxazolyl, pyrazinyl, pyridazinyl, pyridinyl, pyrimidinyl, pyrrolyl, 1,3-thiazolyl, and thienyl wherein the heterocycle is independently substituted with 0, 1, 2, or 3 substituents selected from the group consisting of alkoxy, alkoxycarbonyl, alkyl, cyano, halogen, haloalkoxy, haloalkyl, and nitro;
B is 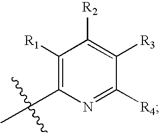
Z is N;
— is absent;
D is —CH₂—; and
L is —N(R₇)C(O)—.

17. The method according to claim 1 wherein
A is heterocycle wherein the heterocycle is pyridinyl independently substituted with 0, 1, 2, or 3 substituents selected from the group consisting of alkoxy, alkoxycarbonyl, alkyl, cyano, halogen, haloalkoxy, haloalkyl, and nitro;
B is 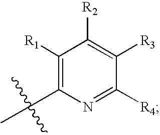
R₁ is selected from the group consisting of hydrogen, alkyl, cyano, and halogen;
R₂, R₃, and R₄ are hydrogen;
Z is N;
— is absent;
D is —CH₂—; and
L is —N(R₇)C(O)—.

18. The method according to claim 17 wherein the compound of formula (I) is 2-[4-(3-cyano-2-pyridinyl)-1-piperazinyl]-N-3-pyridinylacetamide.

19. The method according to claim 1 wherein
A is cycloalkyl;
B is 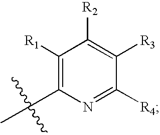
Z is N;
— is absent; and
L is —N(R₇)C(O)—.

20. The method according to claim 1 wherein
A is cycloalkyl wherein the cycloalkyl is selected from the group consisting of cyclohexyl and adamantyl wherein the cycloalkyl is independently substituted with 0, 1, 2, or 3 substituents selected from the group consisting of alkoxy, alkoxycarbonyl, alkyl, halogen, haloalkoxy, and haloalkyl;
B is 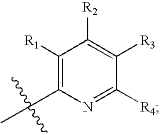
R₁ is selected from the group consisting of hydrogen, alkyl, cyano, and halogen;
R₂, R₃, and R₄ are hydrogen;
Z is N;
— is absent;
D is —CH₂—; and
L is —N(R₇)C(O)—.

21. The method according to claim 1 wherein
A is arylalkyl;
B is 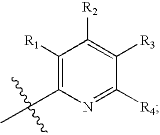
Z is N;
— is absent; and
L is —N(R₇)C(O)—.

22. The method according to claim 1 wherein
A is arylalkyl wherein the aryl of arylalkyl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 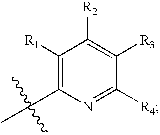
R₁ is selected from the group consisting of hydrogen, alkyl, cyano, and halogen;
R₂, R₃, and R₄ are hydrogen;
Z is N;
— is absent;
D is —CH₂—; and
L is —N(R₇)C(O)—.

23. The method according to claim 1 wherein
A is aryl;
B is 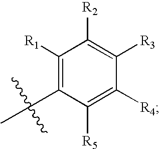
Z is CH;
— is absent; and
L is —N(R₇)C(O)—.

24. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 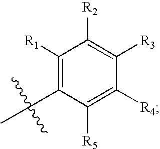
R₁ is selected from the group consisting of hydrogen, alkoxy, alkyl, and halogen;
R₂ is selected from the group consisting of hydrogen and halogen;
R₃, R₄, and R₅ are hydrogen;
Z is CH;
— is absent;
D is —CH₂—; and
L is —N(R₇)C(O)—.

25. The method according to claim 24 wherein the compound of formula (I) is selected from the group consisting of
2-[4-(2-methoxyphenyl)-1-piperidinyl]-N-(3-methylphenyl)acetamide;
2-[4-(2-fluorophenyl)-1-piperidinyl]-N-(3-methylphenyl)acetamide;
N-(3-methylphenyl)-2-[4-(2-methylphenyl)-1-piperidinyl]acetamide; and
2-[4-(3-fluorophenyl)-1-piperidinyl]-N-(3-methylphenyl)acetamide.

26. The method according to claim 1 wherein
A is aryl;
B is 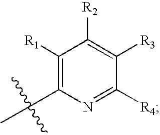
Z is CH;
— is absent; and
L is —N(R₇)C(O)—.

27. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 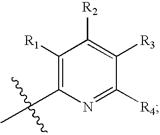
R₁ is selected from the group consisting of hydrogen, alkyl, cyano, and halogen;
R₂, R₃, and R₄ are hydrogen;
Z is CH;
— is absent;
D is —CH₂—; and
L is —N(R₇)C(O)—.

28. The method according to claim 27 wherein the compound of formula (I) is selected from the group consisting of
N-(4-bromophenyl)-2-[4-(2-pyridinyl)-1-piperidinyl]acetamide;
N-(2,6-dimethylphenyl)-2-[4-(2-pyridinyl)-1-piperidinyl]acetamide;
N-(2-nitrophenyl)-2-[4-(2-pyridinyl)-11-piperidinyl]acetamide;
N-(3-nitrophenyl)-2-[4-(2-pyridinyl)-1-piperidinyl]acetamide;
N-(2,4-difluorophenyl)-2-[4-(2-pyridinyl)-1-piperidinyl]acetamide;
N-(2,5-dimethylphenyl)-2-[4-(2-pyridinyl)-1-piperidinyl]acetamide;
N-(2-methylphenyl)-2-[4-(2-pyridinyl)-1-piperidinyl]acetamide;
N-(4-methylphenyl)-2-[4-(2-pyridinyl)-1-piperidinyl]acetamide;
2-[4-(2-pyridinyl)-1-piperidinyl]-N-[3-(trifluoromethyl)phenyl]acetamide;
ethyl 4-({[4-(2-pyridinyl)-1-piperidinyl]acetyl}amino)benzoate;
N-(3-chloro-4-methylphenyl)-2-[4-(2-pyridinyl)-1-piperidinyl]acetamide;
N-(2-cyanophenyl)-2.[4-(2-pyridinyl)-1-piperidinyl]acetamide;
N-(3-chlorophenyl)-2-[4-(2-pyridinyl)-1-piperidinyl]acetamide;
2-[4-(3-cyano-2-pyridinyl)-1-piperidinyl]-N-(3-methylphenyl)acetamide;
N-(4-fluorophenyl)-2-[4-(2-pyridinyl)-1-piperidinyl]acetamide;
N-(3,5-dichlorophenyl)-2-[4-(2-pyridinyl)-1-piperidinyl]acetamide;
N-(2,3-dichlorophenyl)-2-[4-(2-pyridinyl)-1-piperidinyl]acetamide;
2-[4-(2-pyridinyl)-1-piperidinyl]-N-[2-(trifluoromethyl)phenyl]acetamide;
N-(3-chloro-4-fluorophenyl)-2-[4-(2-pyridinyl)-1-piperidinyl]acetamide; and
2-[4-(2-pyridinyl)-1-piperidinyl]-N-[4-(trifluoromethoxy)phenyl]acetamide.

29. The method according to claim 27 wherein the compound of formula (I) is N-(3-methylphenyl)-2-[4-(2-pyridinyl)-1-piperidinyl]acetamide.

30. The method according to claim 1 wherein
A is aryl;
B is 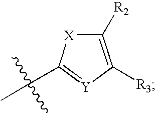
Z is CH;
— is absent; and
L is —N(R₇)C(O)—

31. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 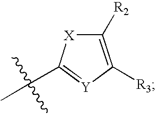
X is S;
Y is N;
R₂ and R₃ are hydrogen;
Z is CH;
— is absent;
D is —CH₂—; and
L is —N(R₇)C(O)—.

32. The method according to claim 31 wherein the compound of formula (I) is selected from the group consisting of
N-(2,6-dimethylphenyl)-2-[4-(2-thienyl)-1-piperidinyl]acetamide;
N-(2,5-dimethylphenyl)-2-[4-(2-thienyl)-1-piperidinyl]acetamide;
N-(2-methylphenyl)-2-[4-(2-thienyl)-1-piperidinyl]acetamide; and
N-(3-chloro-4-fluorophenyl)-2-[4-(2-thienyl)-1-piperidinyl]acetamide.

33. The method according to claim 1 wherein
A is aryl;
B is 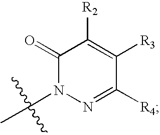
Z is CH;
— is absent; and
L is —N(R₇)C(O)—.

34. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 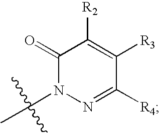
Z is CH;
— is absent;
L is —N(R₇)C(O)—;
D is —CH₂—; and
R₂, R₃, and R₄ are hydrogen.

35. The method according to claim 34 wherein the compound of formula (I) is N-(3-methylphenyl)-2-[4-(6-oxo-1 (6H)-pyridazinyl)-1-piperidinyl] acetamide.

36. The method according to claim 1 wherein
A is aryl;
B is 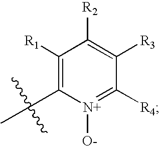
Z is CH;
— is absent; and
L is —N(R₇)C(O)—.

37. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 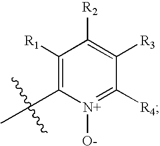
R₁, R₂, R₃, and R₄ are hydrogen;
Z is CH;
— is absent;
D is —CH₂—; and
L is —N(R₇)C(O)—.

38. The method according to claim 37 wherein the compound of formula (I) is 2-(1-{2-[(3-methylphenyl)amino]-2-oxoethyl}piperidin-4-yl)pyridiniumn N-oxide.

39. The method according to claim 1 wherein
A is cycloalkyl;
B is 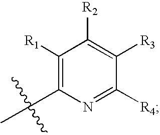
Z is CH;
— is absent; and
L is —N(R₇)C(O)—.

40. The method according to claim 1 wherein
A is cycloalkyl wherein the cycloalkyl is selected from the group consisting of cyclohexyl and adamantyl wherein the cycloalkyl is independently substituted with 0, 1, 2, or 3 substituents selected from the group consisting of alkoxy, alkoxycarbonyl, alkyl, halogen, haloalkoxy, and haloalkyl;
B is 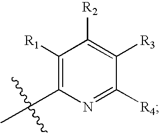
R₁ is selected from the group consisting of hydrogen, alkyl, cyano, and halogen;
R₂, R₃, and R₄ are hydrogen;
Z is CH;
— is absent;
D is —CH₂—; and
L is —N(R₇)C(O)—.

41. The method according to claim 40 wherein the compound of formula (I) is N-cyclohexyl-2-(3′,4′,5′,6′-tetrahydro-2′H-[2,4′]bipyridinyl-1′-yl) acetamide.

42. The method according to claim 1 wherein
A is aryl;
B is 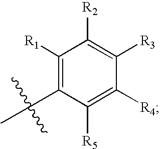
Z is C;
— is a bond; and
L is —N(R₇)C(O)—.

43. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 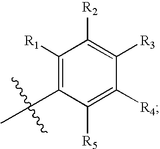
R₁ is selected from the group consisting of hydrogen, alkoxy, alkyl, cyano, halogen, and nitro;
R₂ is selected from the group consisting of hydrogen, cyano, and halogen;
R₃, R₄, and R₅ are hydrogen;
Z is C;
—-is a bond;
D is —CH₂—; and
L is —N(R₇)C(O)—.

44. The method according to claim 43 wherein the compound of formula (I) is N-(3-methylphenyl)-2-(4-phenyl-3,6-dihydro-1(2H)-pyridinyl)acetamide.

45. The method according to claim 1 wherein
A is aryl;
B is 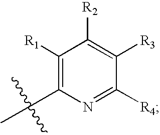
Z is C;
— is a bond; and
L is —N(R₇)C(O)—.

46. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 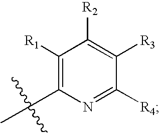
R₁ is selected from the group consisting of hydrogen, alkyl, cyano, and halogen;
R₂, R₃, and R₄ are hydrogen;
Z is C;
— is a bond;
D is —CH₂—; and
L is —N(R₇)C(O)—.

47. The method according to claim 46 wherein the compound of formula (I) is selected from the group consisting of
2-(3′,6′-dihydro-2,4′-bipyridin-1′(2′H)-yl)-N-(3-methylphenyl)acetamide;
2-(3′,6′-dihydro-2,4′-bipyridin-1′(2′H)-yl)-N-(2,6-dimethylphenyl)acetamide;
2-(3′,6′-dihydro-2,4′-bipyridin-1′(2′H)-yl)-N-(2-nitrophenyl)acetamide;
2-(3′,6′-dihydro-2,4′-bipyridin-1′(2′H)-yl)-N-(3-nitrophenyl)acetamide;
2-(3′,6′-dihydro-2,4′-bipyridin-1′(2′H)-yl)-N-(4-fluorophenyl)acetamide;
N-(2,4-difluorophenyl)-2-(3′,6′-dihydro-2,4′-bipyridin-1′(2′H)-yl)acetamide;
2-(3′,6′-dihydro-2,4′-bipyridin-1′(2′H)-yl)-N-(2,5-dimethylphenyl)acetamide;
2-(3′,6′-dihydro-2,4′-bipyridin-1′(2′H)-yl)-N-(2-methylphenyl)acetamide;
2-(3′,6′-dihydro-2,4′-bipyridin-1′(2′H)-yl)-N-(4-methylphenyl)acetamide;
2-(3′,6′-dihydro-2,4′-bipyridin-1′(2′H)-yl)-N-[3-(trifluoromethyl)phenyl]acetamide;
ethyl 4-[(3′,6′-dihydro-2,4′-bipyridin-1′(2′H)-ylacetyl)amino]benzoate;
N-[2-chloro-5-(trifluoromethyl)phenyl]-2-(3′,6′-dihydro-2,4′-bipyridin-1′(2′H)-yl)acetamide;
N-(3-chloro-4-methylphenyl)-2-(3′,6′-dihydro-2,4′-bipyridin-1′(2′H)-yl)acetamide;
N-(2-cyanopheny)-2-(3′,6′-dihydro-2,4′-bipyridin-1′(2′H)-yl)acetamide;
N-(3-chlorophenyl)-2-(3′,6′-dihydro-2,4′-bipyridin-1′(2′H)-yl)acetamide;
N-(3-chloro-4-fluorophenyl)-2-(3′,6′-dihydro-2,4′-bipyridin-1′(2)-yl)acetamide;
2-(3′,6′-dihiydro-2,4′-bipyridin-1′(2′H)-yl)-N-[4-(trifluoromethoxy)phenyl]acetamide;
2-(3′,6′-dihydro-2,4′-bipyridin-(2′H)yl)N[2-(trifluoromethyl)phenyl]acetamide;
N-(4-chlorophenyl)-2-(3′,6′-dihydro-2,4′-bipyridin 1′(2H)-yl) acetamide;
N-(2,3-dichlorophenyl)-2-(3′,6′-dihydro-2,4′-bipyridin-1′(2′H)-yl)acetamide;
N-(3,5-dichlorophenyl)-2-(3′,6′-dihydro-2,4′-bipyridin-1′(2′H)-yl)acetamide; and
2-(3′,6′-dihydro-2,4′-bipyridin-1′(2′H)-yl)-N-(4-fluoro-2-methylphenyl)acetamide.

48. The method according to claim 1 wherein
A is aryl;
B is 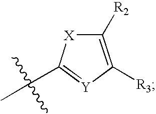
Z is C;
— is a bond;
L is —N(R₇)C(O)—.

49. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 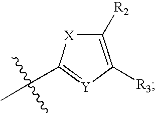
X is S;
Y is C(R₄);
R₂ and R₃ are hydrogen;
R₄ is selected from the group consisting of hydrogen and cyano;
Z is C;
—-is a bond;
D is —CH₂—; and
L is —N(R₇)C(O)—.

50. The method according to claim 1 wherein
A is cycloalkyl;
B is 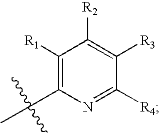
Z is C;
— is a bond; and
L is —N(R₇)C(O)—.

51. The method according to claim 1 wherein
A is cycloalkyl wherein the cycloalkyl is selected from the group consisting of cyclohexyl and adamantyl wherein the cycloalkyl is independently substituted with 0, 1, 2, or 3 substituents selected from the group consisting of alkoxy, alkoxycarbonyl, alkyl, halogen, haloalkoxy, and haloalkyl;
B is 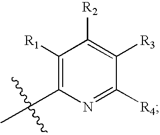
R₁ is selected from the group consisting of hydrogen, alkyl, cyano, and halogen;
R₂, R₃, and R₄ are hydrogen;
Z is C;
— is a bond;
D is —CH₂—; and
L is —N(R₇)C(O)—.

52. The method according to claim 51 wherein the compound of formula (I) is N-cyclohexyl-2-(3′,6′-dihydro-2,4′-bipyridin-1′(2′H)-yl)acetamide.

53. The method according to claim 1 wherein
A is aryl;
B is 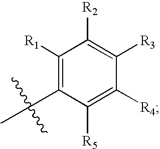
Z is N;
— is absent; and
L is —C(O)N(R₇)—.

54. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 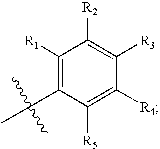
R₁ is selected from the group consisting of hydrogen, alkoxy, alkyl, cyano, halogen, and nitro;
R₂ is selected from the group consisting of hydrogen, cyano, and halogen;
R₃, R₄, and R₅ are hydrogen;
Z is N;
— is absent;
D is —CH₂—; and
L is —C(O)N(R₇)—.

55. The method according to claim 54 wherein the compound of formula (I) is selected from the group consisting of
N-{[4-(2-cyanophenyl)-1-piperazinyl]methyl}-3-methylbenzamide;
3-methyl-N-[(4-phenyl-1-piperazinyl)methyl]benzamide;
N-{[4-(2-methoxyphenyl)-1-piperazinyl]methyl}-3-methylbenzamide;
N-{[4-(2-cyanophenyl)-1-piperazinyl]methyl}-2-methylbenzamide;
N-{[4-(2-cyanophenyl)-1-piperazinyl]methyl}-4-methylbenzamide;
N-{[4-(3-cyanophenyl)-1-piperazinyl]methyl}-3-methylbenzamide;
N-{[4-(3-cyanophenyl)-1-piperazinyl]methyl}-2-methylbenzamide;
N-{[4-(2-chlorophenyl)-1-piperazinyl]methyl}benzamide;
3-chloro-N-{[4-(2-cyanophenyl)-1-piperazinyl]methyl}benzamide;
4-chloro-N-{[4-(2-methoxyphenyl)-1-piperazinyl]methyl}benzamide; and
N-{[4-(2-cyanophenyl)-1-piperazinyl]methyl}benzamide.

56. The method according to claim 1 wherein
A is aryl;
B is 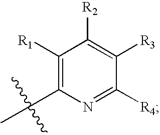
Z is N;
— is absent; and
L is —C(O)N(R₇)—.

57. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 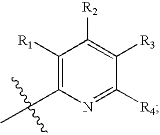
R₁ is selected from the group consisting of hydrogen, alkyl, cyano, and halogen;
R₂, R₃, and R₄ are hydrogen;
Z is N;
— is absent;
D is —CH₂—; and
L is —C(O)N(R₇)—.

58. The method according to claim 57 wherein the compound of formula (I) is selected from the group consisting of
3-methyl-N-{[4-(2-pyridinyl)-1-piperazinyl]methyl}benzamide;
N-{[4-(3-cyano-2-pyridinyl)-1-piperazinyl]methyl}-3-methylbenzamide;
N-{[4-(3-cyano-2-pyridinyl)-1-piperazinyl]methyl}benzamide;
N-{[4-(3-cyano-2-pyridinyl)-1-piperazinyl]methyl}-4-methylbenzamide;
N-{[4-(3-cyano-2-pyridinyl)-1-piperazinyl]methyl}-2-methylbenzamide;
N-{[4-(2-pyridinyl)-1-piperazinyl]methyl}benzamide;
2-chloro-N-{[4-(3-cyano-2-pyridinyl)-1-piperazinyl]methyl}benzamide; and
N-{[4-(3-cyano-2-pyridinyl)-1-piperazinyl]methyl}-2-(trifluoromethyl)benzamide.

59. The method according to claim 1 wherein
A is aryl;
B is 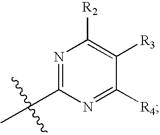
Z is N;
— is absent; and
L is —C(O)N(R₇)—.

60. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 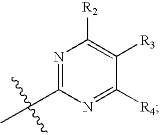
R₂, R₃, and R₄ are hydrogen;
Z is N;
— is absent;
D is —CH₂—; and
L is —C(O)N(R₇)—.

61. The method according to claim 60 wherein the compound of formula (I) is 3-methyl-N-{[4-(2-pyrimidinyl)-1-piperazinyl]methyl}benzamide.

62. The method according to claim 1 wherein
A is cycloalkyl;
B is 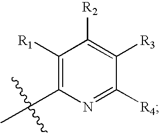
Z is N;
— is absent; and
L is —C(O)N(R₇)—.

63. The method according to claim 1 wherein
A is cycloalkyl wherein the cycloalkyl is selected from the group consisting of cyclohexyl and adamantyl wherein the cycloalkyl is independently substituted with 0, 1, 2, or 3 substituents selected from the group consisting of alkoxy, alkoxycarbonyl, alkyl, halogen, haloalkoxy, and haloalkyl;
B is 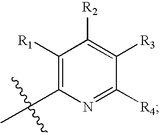
R₁ is selected from the group consisting of hydrogen, alkyl, cyano, and halogen;
R₂, R₃, and R₄ are hydrogen;
Z is N;
— is absent;
D is —CH₂—; and
L is —C(O)N(R₇)—.

64. The method according to claim 1 wherein
A is aryl;
B is 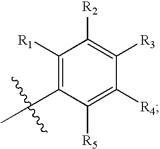
Z is CH;
— is absent; and
L is —C(O)N(R₇)—.

65. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 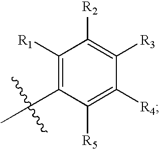
R₁ is selected from the group consisting of hydrogen, alkoxy, alkyl, cyano, halogen, and nitro;
R₂ is selected from the group consisting of hydrogen, cyano, and halogen;
R₃, R₄, and R₅ are hydrogen;
Z is CH;
— is absent;
D is —CH₂—; and
L is —C(O)N(R₇)—.

66. The method according to claim 65 wherein the compound of formula (I) is selected from the group consisting of
N-{[4-(2-methoxyphenyl)-1-piperidinyl]methyl}-3-methylbenzamide; and
3-methyl-N-{[4-(2-pyridinyl)-1-piperidinyl]methyl}benzamide.

67. The method according to claim 1 wherein
A is aryl;
B is 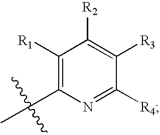
Z is CH;
— is absent; and
L is —C(O)N(R₇)—.

68. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 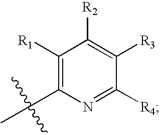
R₁ is selected from the group consisting of hydrogen, alkyl, cyano, and halogen;
R₂, R₃, and R₄ are hydrogen;
Z is CH;
— is absent;
D is —CH₂—; and
L is —C(O)N(R₇)—.

69. The method according to claim 1 wherein
A is aryl;
B is 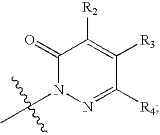
Z is CH;
— is absent; and
L is —C(O)N(R₇)—.

70. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 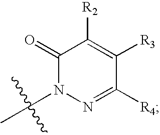
R₂, R₃, and R₄ are hydrogen;
Z is CH;
— is absent;
D is —CH₂—; and
L is —C(O)N(R₇)—.

71. The method according to claim 1 wherein
A is aryl;
B is 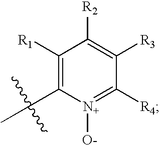
Z is CH;
— is absent; and
L is —C(O)N(R₇)—.

72. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 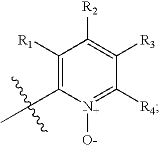
R₁, R₂, R₃, and R₄ are hydrogen;
Z is CH;
— is absent;
D is —CH₂—; and
L is —C(O)N(R₇)—.

73. The method according to claim 1 wherein
A is cycloalkyl;
B is 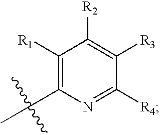
Z is CH;
— is absent; and
L is —C(O)N(R₇)—.

74. The method according to claim 1 wherein
A is cycloalkyl wherein the cycloalkyl is selected from the group consisting of cyclohexyl and adamantyl wherein the cycloalkyl is independently substituted with 0, 1, 2, or 3 substituents selected from the group consisting of alkoxy, alkoxycarbonyl, alkyl, halogen, haloalkoxy, and haloalkyl;
B is 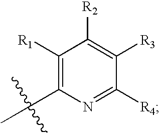
R₁ is selected from the group consisting of hydrogen, alkyl, cyano, and halogen;
R₂, R₃, and R₄ are hydrogen;
Z is CH;
— is absent;
D is —CH₂—; and
L is —C(O)N(R₇)—.

75. The method according to claim 1 wherein
A is aryl;
B is 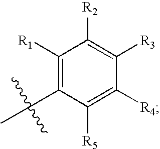
Z is C;
— is a bond; and
L is —C(O)N(R₇)—.

76. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 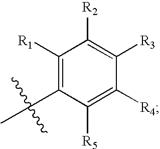
R₁ is selected from the group consisting of hydrogen, alkoxy, alkyl, cyano, halogen, and nitro;
R₂ is selected from the group consisting of hydrogen, cyano, and halogen;
R₃, R₄, and R₅ are hydrogen;
Z is C;
— is a bond;
D is —CH₂—; and
L is —C(O)N(R₇)—.

77. The method according to claim 76 wherein the compound of formula (I) is 3-methyl-N-[(4-phenyl-3,6-dihydro-1 (2H)-pyridinyl)methyl]benzamide.

78. The method according to claim 1 wherein
A is aryl;
B is 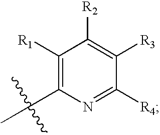
Z is C;
— is a bond; and
L is —C(O)N(R₇)—.

79. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 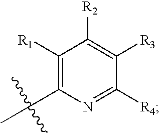
R₁ is selected from the group consisting of hydrogen, alkyl, cyano, and halogen;
R₂, R₃, and R₄ are hydrogen;
Z is C;
— is a bond;
D is —CH₂—; and
L is —C(O)N(R₇)—.

80. The method according to claim 79 wherein the compound of formula (I) is selected from the group consisting of
N-(3′,6′-dihydro-2,4′-bipyridin-1′(2′H)-ylmethyl)-3-methylbenzamide;
N-(3′,6′-dihydro-2,4′-bipyridin-1′(2′H)-ylmethyl)-3-methoxybenzamide;
N-(3′,6′-dihydro-2,4′-bipyridin-1′(2′H)-ylmethyl)-3-fluorobenzamide; and
N-(3′,6′-dihydro-2,4′-bipyridin-1′(2′H)-ylmethyl)-3,5-difluorobenzamide.

81. The method according to claim 1 wherein
A is aryl wherein the aryl is naphthyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 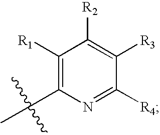
R₁ is selected from the group consisting of hydrogen, alkyl, cyano, and halogen;
R₂, R₃, and R₄ are hydrogen;
Z is C;
— is a bond;
D is —CH₂—; and
L is —C(O)N(R₇)—.

82. The method according to claim 1 wherein
A is aryl;
B is 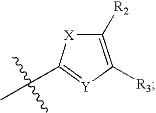
Z is C;
—is a bond; and
L is —C(O)N(R₇)—.

83. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 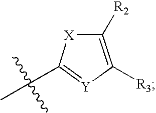
R₂ and R₃ are hydrogen;
X is S;
Y is N;
Z is C;
— is a bond;
D is —CH₂—; and
L is —C(O)N(R₇)—.

84. The method according to claim 1 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 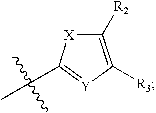
R₂ and R₃ are hydrogen;
X is O;
Y is N;
Z is C;
— is a bond;
D is —CH₂—; and
L is —C(O)N(R₇)—.

85. The method according to claim 1 wherein
A is cycloalkyl;
B is 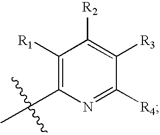
Z is C;
— is a bond; and
L is —C(O)N(R₇)—.

86. The method according to claim 1 wherein
A is cycloalkyl wherein the cycloalkyl is selected from the group consisting of cyclohexyl and adamantyl wherein the cycloalkyl is independently substituted with 0, 1, 2, or 3 substituents selected from the group consisting of alkoxy, alkoxycarbonyl, alkyl halogen, haloalkoxy, and haloalkyl;
B is 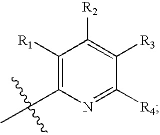
R₁ is selected from the group consisting of hydrogen, alkyl, cyano, and halogen;
R₂, R₃, and R₄ are hydrogen;
Z is C;
is a bond;
D is —CH₂—; and
L is —C(O)N(R₇)—.

87. A method of treating sexual dysfunction in a mammal comprising administering to the mammal a therapeutically effective amount of a compound of formula (I) or a pharmaceutically acceptable salt, ester, amide, or prodrug thereof in combination with a pharmaceutically acceptable carrier.

88. A method of treating sexual dysfunction in a mammal comprising administering to the mammal a therapeutically effective amount of a compound of formula (I) or a pharmaceutically acceptable salt, ester, amide, or prodrug thereof in combination with a phosphodiesterase 5 inhibitor.

89. A method of treating sexual dysfunction in a mammal comprising administering to the mammal a therapeutically effective amount of a compound of formula (I) or a pharmaceutically acceptable salt, ester, amide, or prodrug thereof in combination with an adrenergic receptor antagonist.

90. A method of treating sexual dysfunction in a mammal comprising administering to the mammal a therapeutically effective amount of a compound of formula (I) or a pharmaceutically acceptable salt, ester, amide, or prodrug thereof in combination with a dopamine agonist.

91. A method of treating male erectile dysfunction in a mammal comprising administering to the mammal in need of such treatment a therapeutically effective amount of a compound of formula (I) or a pharmaceutically acceptable salt, ester, amide, or prodrug thereof.

92. A method of treating female sexual dysfunction in a mammal comprising administering to the mammal in need of such treatment a therapeutically effective amount of a compound of formula (I) or a pharmaceutically acceptable salt, ester, amide, or prodrug thereof.

93. A method of treating a disorder selected from the group consisting of cardiovascular disorders, inflammatory disorders, attention deficit hyperactivity disorder, Alzheimer's disease, drug abuse, Parkinson's disease, schizophrenia, anxiety, mood disorders and depression in a mammal comprising administering to the mammal in need of such treatment a therapeutically effective amount of a compound of formula (I) or a pharmaceutically acceptable salt, ester, amide, or prodrug thereof.

94. A compound of formula (II) 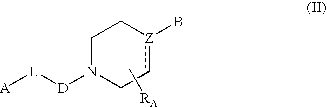
or a pharmaceutically acceptable salt, ester, amide, or prodrug thereof, wherein
A is selected from the group consisting of aryl, arylalkyl, cycloalkyl, and cycloalkylalkyl;
L is selected from the group consisting of —N(R₇)C(O)— and —C(O)N(R₇)— wherein the left end of the —N(R₇)C(O)— and —C(O)N(R₇)— is attached to A and the right end is attached to D;
D is selected from the group consisting of alkylene, fluoroalkylene, and hydroxyalkylene;
R_{A} is selected from the group consisting of hydrogen and alkyl;
Z is selected from the group consisting of N, C and CH;
— is a bond when Z is C and — is absent when Z is N or CH;
B is selected from the group consisting of 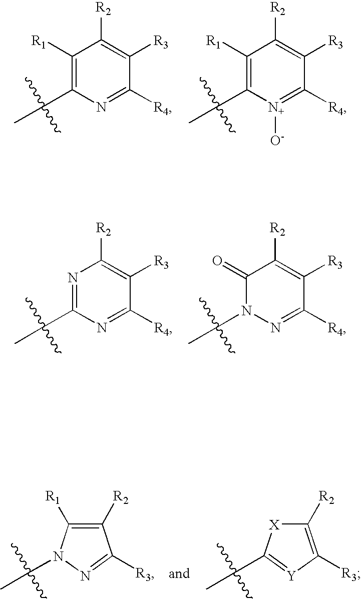
R₁, R₂, R₃, and R₄ are each independently selected from the group consisting of hydrogen, alkoxy, alkenyl, alkyl, alkylsulfinyl, alkylsulfonyl, alkylthio, alkynyl, alkoxycarbonyl, alkylcarbonyl, alkylcarbonyloxy, carboxy, cyano, formyl, halogen, haloalkoxy, haloalkyl, hydroxy, hydroxyalkyl, mercapto, nitro, —NZ₁Z₂, (NZ₃Z₄)carbonyl, and (NZ₃Z₄)sulfonyl;
Z₁ and Z₂ are each independently selected from the group consisting of hydrogen, alkyl, 0.7: alkylcarbonyl, alkylsulfonyl, aryl, arylalkyl, arylalkylsulfonyl, arylsulfonyl, and formyl;
Z₃ and Z₄ are each independently selected from the group consisting of hydrogen, alkyl, aryl, and arylalkyl;
X is selected from the group consisting of N(R₆), O and S;
Y is selected from the group consisting of C(R₄) and N;
R₆ is selected from the group consisting of hydrogen and alkyl; and
R₇ is selected from the group consisting of hydrogen and alkyl.

95. A compound according to claim 94 wherein
A is aryl;
B is 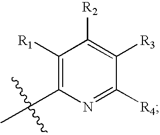
Z is N;
— is absent; and
L is —N(R₇)C(O)—.

96. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 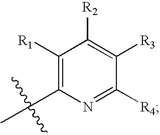
R₁ is selected from the group consisting of hydrogen, alkyl, cyano, and halogen;
R₂, R₃, and R₄ are hydrogen;
Z is N;
— is absent;
D is —CH₂—; and
L is —N(R₇)C(O)—.

97. A compound according to claim 96 wherein the compound of formula (I) is selected from the group consisting of
N-(3-methylphenyl)-2-[4-(2-pyridinyl)-1-piperazinyl]acetamide;
2-[4-(3-cyano-2-pyridinyl)-1-piperazinyl]-N-(3-methylphenyl)acetamide;
2-[4-(3-cyano-2-pyridinyl)-1-piperazinyl]-N-(3-nitrophenyl)acetamide;
2-[4-(3-cyano-2-pyridinyl)-1-piperazinyl]-N-[3-(trifluoromethyl)phenyl]acetamide;
N-(3-cyanophenyl)-2-[4-(3-cyano-2-pyridinyl)-1-piperazinyl]acetamide;
2-[4-(3-cyano-2-pyridinyl)-1-piperazinyl]-N-phenylacetamide;
2-[4-(3-cyano-2-pyridinyl)-1-piperazinyl]-N-(4-fluorophenyl)acetamide;
2-[4-(3-cyano-2-pyridinyl)-1-piperazinyl]-N-(3,5-dimethylphenyl)acetamide;
2-[4-(3-cyano-2-pyridinyl)-1-piperazinyl]-N-(2,3-dimethylphenyl)acetamide;
2-[4-(3-cyano-2-pyridinyl)-1-piperazinyl]-N-(2-methylphenyl)acetamide;
2-[4-(3-cyano-2-pyridinyl)-1-piperazinyl]-N-(2,5-dimethylphenyl)acetamide;
N-(3-chlorophenyl)-2-[4-(3-cyano-2-pyridinyl)-1-piperazinyl] acetamide;
N-(3-chloro-4-fluorophenyl)-2-[4-(3-cyano-2-pyridinyl)-1-piperazinyl]acetamide;
2-[4-(3-cyano-2-pyridinyl)-1-piperazinyl]-N-(3,4,5-trimethoxyphenyl)acetamide;
2-[4-(3-cyano-2-pyridinyl)-1-piperazinyl]-N-[4-fluoro-3-(trifluoromethyl)phenyl]acetamide;
2-[4-(3-cyano-2-pyridinyl)-1-piperazinyl]-N-[3-fluoro-5-(trifluoromethyl)phenyl]acetamide;
2-[4-(3-cyano-2-pyridinyl)-1-piperazinyl]-N-[2-fluoro-5-(trifluoromethyl)phenyl]acetamide;
2-[4-(3-cyano-2-pyridinyl)-1-piperazinyl]-N-[2-fluoro-3-(trifluoromethyl)phenyl]acetamide;
2-[4-(3-cyano-2-pyridinyl)-1-piperazinyl]-N-(4-fluoro-3-methylphenyl)acetamide;
2-[4-(3-cyano-2-pyridinyl)1-piperazinyl]-N-(2-florophenyl)acetamide;
2-[4-(3-cyano-2-pyridinyl)-1-piperazinyl]-N-(2-methoxyphenyl)acetamide;
2-[4-(3-cyano-2-pyridinyl)-1-piperazinyl]-N-(2-nitrophenyl)acetamide;
2-[4-(3-cyano-2-pyridinyl)-1-piperazinyl]-N-[2-(trifluoromethyl)phenyl]acetamide;
N-phenyl-2-[4-(2-pyridinyl)-1-piperazinyl] acetamide; and
2-[4-(3-cyano-2-pyridinyl)-1-piperazinyl]-N-(4-methylphenyl) acetamide.

98. A compound according to claim 94 wherein
A is aryl wherein the aryl is tetrahydronaphthalenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 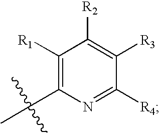
R₁ is selected from the group consisting of hydrogen, alkyl, cyano, and halogen;
R₂, R₃, and R₄ are hydrogen;
Z is N;
— is absent;
D is —CH₂—; and
L is —N(R₇)C(O)—.

99. A compound according to claim 94 wherein
A is aryl;
B is 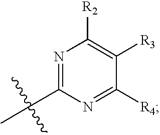
Z is N;
— is absent; and
L is —N(R₇)C(O)—.

100. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 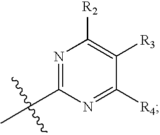
R₂, R₃, and R₄ are hydrogen;
Z is N;
— is absent;
D is —CH₂—; and
L is —N(R₇)C(O)—.

101. A compound according to claim 100 wherein the compound of formula (I) is N-(3-methylphenyl)-2-[4-(2-pyrimidinyl)-1-piperazinyl]acetamide.

102. A compound according to claim 94 wherein
A is aryl;
B is 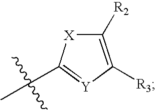
Z is N;
— is absent; and
L is —N(R₇)C(O)—.

103. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 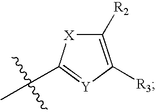
R₂ and R₃ are hydrogen;
X is S;
Y is N;
Z is N;
— is absent;
D is —CH₂—; and
L is —N(R₇)C(O)—.

104. A compound according to claim 103 wherein the compound of formula (I) is N-(3-methylphenyl)-2-[4-(1,3-thiazol-2-yl)-1-piperazinyl]acetamide.

105. A compound according to claim 1 wherein
A is cycloalkyl;
B is 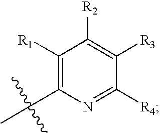
Z is N;
— is absent; and
L is —N(R₇)C(O)—.

106. A compound according to claim 94 wherein
A is cycloalkyl wherein the cycloalkyl is selected from the group consisting of cyclohexyl and adamantyl wherein the cycloalkyl is independently substituted with 0, 1, 2, or 3 substituents selected from the group consisting of alkoxy, alkoxycarbonyl, alkyl, halogen, haloalkoxy, and haloalkyl;
B is 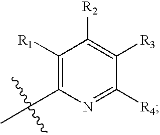
R₁ is selected from the group consisting of hydrogen, alkyl, cyano, and halogen;
R₂, R₃, and R₄ are hydrogen;
Z is N;
— is absent;
D is —CH₂—; and
L is —N(R₇)C(O)—.

107. A compound according to claim 94 wherein
A is arylalkyl;
B is 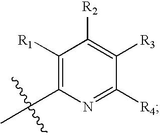
Z is N;
— is absent; and
L is —N(R₇)C(O)—.

108. A compound according to claim 94 wherein
A is arylalkyl wherein the aryl of arylalkyl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 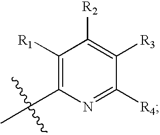
R₁ is selected from the group consisting of hydrogen, alkyl, cyano, and halogen;
R₂, R₃, and R₄ are hydrogen;
Z is N;
— is absent;
D is —CH₂—; and
L is —N(R₇)C(O)—.

109. A compound according to claim 94 wherein
A is aryl;
B is 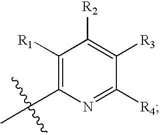
Z is CH;
— is absent; and
L is —N(R₇)C(O)—.

110. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 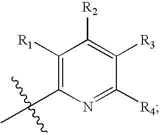
R₁ is selected from the group consisting of hydrogen, alkyl, cyano, and halogen;
R₂, R₃, and R₄ are hydrogen;
Z is CH;
— is absent;
D is —CH₂—; and
L is —N(R₇)C(O)—.

111. A compound according to claim 110 wherein the compound of formula (I) is selected from the group consisting of
N-(4-bromophenyl)-2-[4-(2-pyridinyl)-1-piperidinyl]acetamide;
N-(2,6-dimethylphenyl)-2-[4-(2-pyridinyl)-1-piperidinyl]acetamide;
N-(2-nitrophenyl)-2-[4-(2-pyridinyl)-1-piperidinyl]acetamide;
N-(3-nitrophenyl)-2-[4-(2-pyridinyl)-1-piperidinyl]acetamide;
N-(2,4-difluorophenyl)-2-[4-(2-pyridinyl)-1-piperidinyl]acetamide;
N-(2,5-dimethylphenyl)-2-[4-(2-pyridinyl)-1-piperidinyl]acetamide;
N-(2-methylphenyl)-2-[4-(2-pyridinyl)-1-piperidinyl]acetamide;
N-(4-methylphenyl)-2-[4-(2-pyridinyl)-1-piperidinyl]acetamide;
2-[4-(2-pyridinyl)-1-piperidinyl]-N-[3-(trifluoromethyl)phenyl]acetamide;
ethyl 4-({[4-(2-pyridinyl)-1-piperidinyl]acetyl}amino)benzoate;
N-(3-chloro-4-methylphenyl)-2-[4-(2-pyridinyl)-1-piperidinyl]acetamide;
N-(2-cyanophenyl)-2-[4-(2-pyridinyl)-1-piperidinyl]acetamide;
N-(3-chlorophenyl)-2-[4-(2-pyridinyl)-1-piperidinyl]acetamide;
2-[4-(3-cyano-2-pyridinyl)-1-piperidinyl]-N-(3-methylphenyl)acetamide;
N-(4-fluorophenyl)-2-[4-(2-pyridinyl)-1-piperidinyl]acetamide;
N-(3,5-dichlorophenyl)-2-[4-(2-pyridinyl)-1-piperidinyl]acetamide;
N-(2,3-dichlorophenyl)-2-[4-(2-pyridinyl)-1-piperidinyl]acetamide;
2-[4-(2-pyridinyl)-1-piperidinyl]-N-[2-(trifluoromethyl)phenyl]acetamide;
N-(3-chloro-4-fluorophenyl)-2-[4-(2-pyridinyl)-1-piperidinyl]acetamide; and
2-[4-(2-pyridinyl)-1-piperidinyl]-N-[4-(trifluoromethoxy)phenyl]acetamide.

112. A compound according to claim 111 wherein the compound of formula (I) is N-(3-methylphenyl)-2-[4-(2-pyridinyl)-1-piperidinyl]acetamide.

113. A compound according to claim 94 wherein
A is aryl;
B is 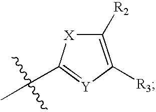
Z is CH;
— is absent;
L is —N(R₇)C(O)—.

114. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 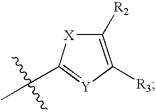
X is S;
Y is N;
R₂ and R₃ are hydrogen;
Z is CH;
— is absent;
D is —CH₂—; and
L is —N(R₇)C(O)—.

115. A compound according to claim 114 wherein the compound of formula (I) is selected from the group consisting of
N-(2,6-dimethylphenyl)-2-[4-(2-thienyl)-1-piperidinyl]acetamide;
N-(2,5-dimethylphenyl)-2-[4-(2-thienyl)-1-piperidinyl]acetamide;
N-(2-methylphenyl)-2-[4-(2-thienyl)-1-piperidinyl]acetamide; and
N-(3-chloro-4-fluorophenyl)-2-[4-(2-thienyl)-1-piperidinyl]acetamide.

116. A compound according to claim 94 wherein
A is aryl;
B is 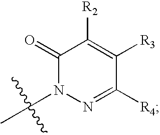
Z is CH;
— is absent; and
L is —N(R₇)C(O)—.

117. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 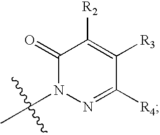
Z is CH;
— is absent;
L is —N(R₇)C(O)—;
D is —CH₂—; and
R₂, R₃, and R₄ are hydrogen.

118. A compound according to claim 117 wherein the compound of formula (I) is N-(3-methylphenyl)-2-[4-(6-oxo-1 (6H)-pyridazinyl)-1-piperidinyl] acetamide.

119. A compound according to claim 94 wherein
A is aryl;
B is 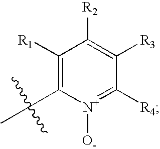
Z is CH;
— is absent; and
L is —N(R₇)C(O)—.

120. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 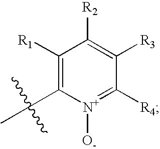
R₁, R₂, R₃, and R₄ are hydrogen;
Z is CH;
— is absent;
D is —CH₂—; and
L is —N(R₇)C(O)—.

121. A compound according to claim 120 wherein the compound of formula (I) is 2-(1-{2-[(3-methylphenyl)amino]-2-oxoethyl}piperidin-4-yl)pyridiniumn N-oxide.

122. A compound according to claim 94 wherein
A is cycloalkyl;
B is 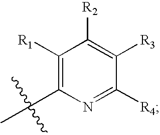
Z is CH;
— is absent; and
L is —N(R₇)C(O)—.

123. A compound according to claim 94 wherein
A is cycloalkyl wherein the cycloalkyl is selected from the group consisting of cyclohexyl and adamantyl wherein the cycloalkyl is independently substituted with 0, 1, 2, or 3 substituents selected from the group consisting of alkoxy, alkoxycarbonyl, alkyl, halogen, haloalkoxy, and haloalkyl;
B is 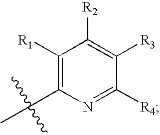
R₁ is selected from the group consisting of hydrogen, alkyl, cyano, and halogen;
R₂, R₃, and R are hydrogen;
Z is CH;
— is absent;
D is —CH₂—; and
L is —N(R₇)C(O)—.

124. A compound according to claim 123 wherein the compound of formula (I) is N-cyclohexyl-2-(3′,4′,5′,6′-tetrahydro-2′H-[2,4′]bipyridinyl-1′-yl) acetamide.

125. A compound according to claim 94 wherein
A is aryl;
B is 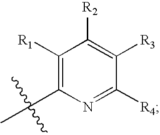
Z is C;
— is a bond; and
L is —N(R₇)C(O)—.

126. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 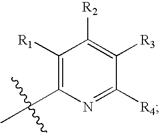
R₁ is selected from the group consisting of hydrogen, alkyl, cyano, and halogen;
R₂, R₃, and R₄ are hydrogen;
Z is C;
is a bond;
D is —CH₂—; and
L is —N(R₇)C(O)—.

127. A compound according to claim 126 wherein the compound of formula (I) is selected from the group consisting of
2-(3′,6′-dihydro-2,4′-bipyridin-1′(2′H)-yl)-N-(3-methylphenyl)acetamide;
2-(3′,6′-dihydro-2,4′-bipyridin-1′(2′H)-yl)-N-(2,6-dimethylphenyl)acetamide;
2-(3′,6′-dihydro-2,4′-bipyridin-1′(2)-yl)-N-(2-nitrophenyl)acetamide;
2-(3′,6′-dihydro-2,4′-bipyridin-1′(2′H)-yl)-N-(3-nitrophenyl)acetamide;
2-(3′,6′-dihydro-2,4′-bipyridin-1′(2′H)-yl)-N-(4-fluorophenyl)acetamide;
N-(2,4-difluorophenyl)-2-(3′,6′-dihydro-2,4′-bipyridin-1′(2′H)-yl)acetamide;
2-(3′,6′-dihydro-2,4′-bipyridin-1′(2′H)-yl)-N-(2,5-dimethylphenyl)acetamide;
2-(3′,6′-dihydro-2,4′-bipyridin-1′(2′H)-yl)-N-(2-methylphenyl)acetamide;
2-(3′,6′-dihydro-2,4′-bipyridin-1′(2′H)-yl)-N-(4-methylphenyl)acetamide;
2-(3′,6′-dihydro-2,4′-bipyridin-1′(2′H)-yl)-N-[3-(trifluoromethyl)phenyl]acetamide;
ethyl 4-[(3′,6′-dihydro-2,4′-bipyridin-1′(2′H)-ylacetyl)amino]benzoate;
N-[2-chloro-5-(trifluoromethyl)phenyl]-2-(3′,6′-dihydro-2,4′-bipyridin-1′(2′H)-yl)acetamide;
N-(3-chloro-4-methylphenyl)-2-(3′,6′-dihydro-2,4′-bipyridin-1′(2′H)-yl)acetamide;
N-(2-cyanophenyl)-2-(3′,6′-dihydro-2,4′-bipyridin-1′(2′H)-yl)acetamide;
N-(3-chlorophenyl)-2-(3′,6′-dihydro-2,4′-bipyridin-1′(2′H)-yl)acetamide;
N-(3-chloro-4-fluorophenyl)-2-(3′,6′-dihydro-2,4′-bipyridin-1′(2′H)-yl)acetamide;
2-(3′,6′-dihydro-2,4′-bipyridin-1′(2′H)-yl)-N-[4-(trifluoromethoxy)phenyl]acetamide;
2-(3′,6′-dihydro-2,4′-bipyridin-1′(2′H)-yl)-N-[2-(trifluoromethyl)phenyl]acetamide;
N-(4-chlorophenyl)-2-(3′,6′-dihydro-2,4′-bipyridin-1′(2′H)-yl)acetamide;
N-(2,3-dichlorophenyl)-2-(3′,6′-dihydro-2,4′-bipyridin-1′(2′H)-yl)acetamide;
N-(3,5-dichlorophenyl)-2-(3′,6′-dihydro-2,4′-bipyridin-1′(2′H)-yl)acetamide; and
2-(3′,6′-dihydro-2,4′-bipyridin-1′(2′H)-yl)-N-(4-fluoro-2-methylphenyl)acetamide.

128. A compound according to claim 94 wherein
A is aryl;
B is 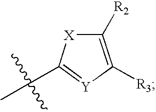
Z is C;
— is a bond; and
L is —N(R₇)C(O)—.

129. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 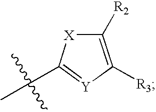
X is S;
Y is C(R₄);
R₂ and R₃ are hydrogen;
R₄ is selected from the group consisting of hydrogen and cyano;
Z is C;
— is a bond;
D is —CH₂—; and
L is —N(R₇)C(O)—.

130. A compound according to claim 94 wherein
A is cycloalkyl;
B is 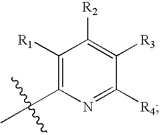
Z is C;
— is a bond; and
L is —N(R₇)C(O)—.

131. A compound according to claim 94 wherein
A is cycloalkyl wherein the cycloalkyl is selected from the group consisting of cyclohexyl and adamantyl wherein the cycloalkyl is independently substituted with 0, 1, 2, or 3 substituents selected from the group consisting of alkoxy, alkoxycarbonyl, alkyl, halogen, haloalkoxy, and haloalkyl;
B is 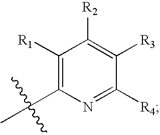
R₁ is selected from the group consisting of hydrogen, alkyl, cyano, and halogen;
R₂, R₃, and R₄ are hydrogen;
Z is C;
— is a bond;
D is —CH₂—; and
L is —N(R₇)C(O)—.

132. A compound according to claim 131 wherein the compound of formula (I) is N-cyclohexyl-2-(3′,6′-dihydro-2,4′-bipyridin-1′(2′H)-yl)acetamide.

133. A compound according to claim 94 wherein
A is aryl;
B is 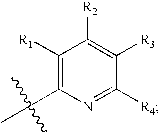
Z is N;
— is absent; and
L is —C(O)N(R₇)—.

134. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 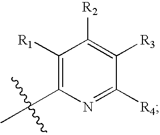
R₁ is selected from the group consisting of hydrogen, alkyl, cyano, and halogen;
R₂, R₃, and R₄ are hydrogen;
Z is N;
— is absent;
D is —CH₂—; and
L is —C(O)N(R₇)—.

135. A compound according to claim 134 wherein the compound of formula (I) is selected from the group consisting of
3-methyl-N-{[4-(2-pyridinyl)-1-piperazinyl]methyl}benzamide;
N-{[4-(3-cyano-2-pyridinyl)-1-piperazinyl]methyl}-3-methylbenzamide;
N-{[4-(3-cyano-2-pyridinyl)-1-piperazinyl]methyl}benzamide;
N-{[4-(3-cyano-2-pyridinyl)-1-piperazinyl]methyl}-4-methylbenzamide;
N-{[4-(3-cyano-2-pyridinyl)-1-piperazinyl]methyl}-2-methylbenzamide;
N-{[4-(2-pyridinyl)-1-piperazinyl]methyl}benzamide;
2-chloro-N-{[4-(3-cyano-2-pyridinyl)-1-piperazinyl]methyl}benzamide; and
N-{[4-(3-cyano-2-pyridinyl)-1-piperazinyl]methyl}-2-(trifluoromethyl)benzamide.

136. A compound according to claim 94 wherein
A is aryl;
B is 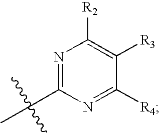
Z is N;
— is absent; and
L is —C(O)N(R₇)—.

137. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 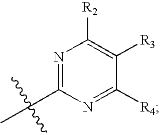
R₂, R₃, and R₄ are hydrogen;
Z is N;
— is absent;
D is —CH₂—; and
L is —C(O)N(R₇)—.

138. A compound according to claim 137 wherein the compound of formula (I) is 3-methyl-N-{[4-(2-pyrimidinyl)-1-piperazinyl]methyl}benzamide.

139. A compound according to claim 94 wherein
A is cycloalkyl;
B is 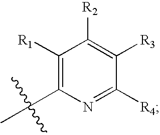
Z is N;
— is absent; and
L is —C(O)N(R₇)—.

140. A compound according to claim 94 wherein
A is cycloalkyl wherein the cycloalkyl is selected from the group consisting of cyclohexyl and adamantyl wherein the cycloalkyl is independently substituted with 0, 1, 2, or 3 substituents selected from the group consisting of alkoxy, alkoxycarbonyl, alkyl, halogen, haloalkoxy, and haloalkyl;
B is 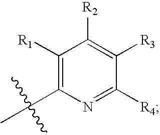
R₁ is selected from the group consisting of hydrogen, alkyl, cyano, and halogen;
R₂, R₃, and R₄ are hydrogen;
Z is N;
— is absent;
D is —CH₂—; and
L is —C(O)N(R₇)—.

141. A compound according to claim 94 wherein
A is aryl;
B is 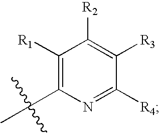
Z is CH;
— is absent; and
L is —C(O)N(R₇)—.

142. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 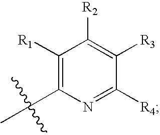
R₁ is selected from the group consisting of hydrogen, alkyl, cyano, and halogen;
R₂, R₃, and R₄ are hydrogen;
Z is CH;
— is absent;
D is —CH₂—; and
L is —C(O)N(R₇)—.

143. A compound according to claim 94 wherein
A is aryl;
B is 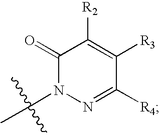
Z is CH;
— is absent; and
L is —C(O)N(R₇)—.

144. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 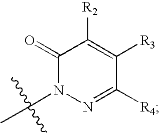
R₂, R₃, and R₄ are hydrogen;
Z is CH;
— is absent;
D is —CH₂—; and
L is —C(O)N(R₇)—.

145. A compound according to claim 94 wherein
A is aryl;
B is 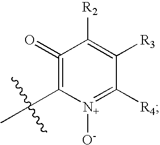
Z is CH;
— is absent; and
L is —C(O)N(R₇)—.

146. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 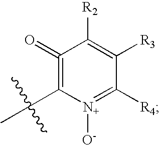
R₁, R₂, R₃, and R₄ are hydrogen;
Z is CH;
— is absent;
D is —CH₂—; and
L is —C(O)N(R₇)—.

147. A compound according to claim 94 wherein
A is cycloalkyl;
B is 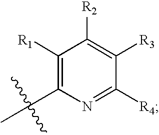
Z is CH;
— is absent; and
L is —C(O)N(R₇)—.

148. A compound according to claim 94 wherein
A is cycloalkyl wherein the cycloalkyl is selected from the group consisting of cyclohexyl and adamantyl wherein the cycloalkyl is independently substituted with 0, 1, 2, or 3 substituents selected from the group consisting of alkoxy, alkoxycarbonyl, alkyl, halogen, haloalkoxy, and haloalkyl;
B is 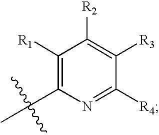
R₁ is selected from the group consisting of hydrogen, alkyl, cyano, and halogen;
R₂, R₃, and R₄ are hydrogen;
Z is CH;
— is absent;
D is —CH₂—; and
L is —C(O)N(R₇)—.

149. A compound according to claim 94 wherein
A is aryl;
B is 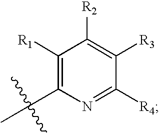
Z is C;
—-is a bond; and
L is —C(O)N(R₇)—.

150. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 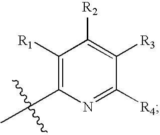
R₁ is selected from the group consisting of hydrogen, alkyl, cyano, and halogen;
R₂, R₃, and R₄ are hydrogen;
Z is C;
— is a bond;
D is —CH₂—; and
L is —C(O)N(R₇)—.

151. A compound according to claim 150 wherein the compound of formula (I) is selected from the group consisting of
N-(3′,6′:dihydro-2,4′-bipyridin-1′(2′H)-ylmethyl)-3-methylbenzamide;
N-(3′,6′-dihydro-2,4′-bipyridin-1′(2′H)-ylmethyl)-3-methoxybenzamide;
N-(3′,6′-dihydro-2,4′-bipyridin-1′(2H)-ylmethyl)-3-fluorobenzamide; and
N-(3′,6′-dihydro-2,4′-bipyridin-1′(2′H)-ylmethyl)-3,5-difluorobenzamide.

152. A compound according to claim 94 wherein
A is aryl wherein the aryl is naphthyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 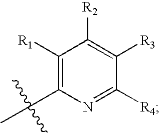
R₁ is selected from the group consisting of hydrogen, alkyl, cyano, and halogen;
R₂, R₃, and R₄ are hydrogen;
Z is C;
— is a bond;
D is —CH₂—; and
L is —C(O)N(R₇)—.

153. A compound according to claim 94 wherein
A is aryl;
B is 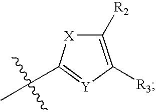
Z is C;
— is a bond; and
L is —C(O)N(R₇)—.

154. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 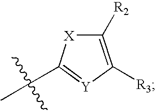
R₂ and R₃ are hydrogen;
X is S;
Y is N;
Z is C;
— is a bond;
D is —CH₂—; and
L is —C(O)N(R₇)—.

155. A compound according to claim 94 wherein
A is aryl wherein the aryl is phenyl substituted with 0, 1, 2, 3, 4, or 5 substituents selected from the group consisting of alkoxy, alkoxycarbonyl, alkyl, cyano, halogen, haloalkoxy, haloalkyl, and nitro;
B is 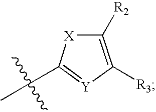
R₂ and R₃ are hydrogen;
X is O;
Y is N;
Z is C;
— is a bond;
D is —CH₂—; and
L is —C(O)N(R₇)—.

156. A compound according to claim 94 wherein
A is cycloalkyl;
B is 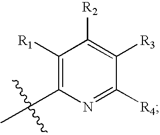
Z is C;
— is a bond; and
L is —C(O)N(R₇)—.

157. A compound according to claim 94 wherein
A is cycloalkyl wherein the cycloalkyl is selected from the group consisting of cyclohexyl and adamantyl wherein the cycloalkyl is independently substituted with 0, 1, 2, or 3 substituents selected from the group consisting of alkoxy, alkoxycarbonyl, alkyl, halogen, haloalkoxy, and haloalkyl;
B is 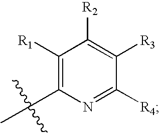
R₁ is selected from the group consisting of hydrogen, alkyl, cyano, and halogen;
R₂, R₃, and R₄ are hydrogen;
Z is C;
— is a bond;
D is —CH₂—; and
L is —C(O)N(R₇)—.
